(19) United States
(12) Patent Application Publication
(10) Pub. No.: US 2025/0134825 A1
(43) Pub. Date: May 1, 2025

(54) METHOD FOR PRODUCING MEDICATIONS AND VACCINES

(71) Applicant: MSLsolutions GmbH, Oberhausen (DE)

(72) Inventor: Gerd Herbert DAHMS, Duisburg (DE)

(21) Appl. No.: 18/834,915

(22) PCT Filed: Feb. 2, 2023
(86) PCT No.: PCT/EP2023/052618

§ 371 Date: Jul. 31, 2024

(30) Foreign Application Priority Data
Feb. 2, 2022 (DE) 10 2022 102 399.0
Apr. 8, 2022 (DE) 10 2022 108 643.7
Jun. 23, 2022 (DE) 10 2022 115 653.2

(51) Int. Cl. A61K 9/51; A61K 31/375; A61K 31/7105; A61K 39/00; A61K 39/385

(52) U.S. Cl. CPC A61K 9/5192; A61K 9/5123; A61K 9/5146; A61K 31/375; A61K 31/7105; A61K 39/385; A61K 2039/53

(57) ABSTRACT

The present invention relates to a method for producing a preferably lipid-based carrier system as well as to a corresponding carrier system, in addition to a composition or corresponding medicament based thereon, as well as to corresponding uses of the composition or medicant.

### CROSS-REFERENCES TO RELATED APPLICATIONS

This application is a National Stage filing of International Application PCT/EP 2023/052618 (WO 2023/148303) filed Feb. 2, 2023, entitled “ENTITLED “METHOD FOR PRODUCING MEDICATIONS AND VACCINES” claiming priority to: DE 10 2022 102 399.0 filed Feb. 2, 2022, DE 10 2022 108 643.7 filed Apr. 8, 2022, and DE 10 2022 115 653.2 filed Jun. 23, 2022. The subject application claims priority to DE 10 2022 102 399.0, DE 10 2022 108 643.7, DE 10 2022 115 653.2 and PCT/EP 2023/052618 and incorporates all by reference herein, in their entirety.

### BACKGROUND OF THE INVENTION

The present invention relates to the technical field of lipid-based systems, which can be used, for example, in the medical field and in particular for the administration or application of, in particular, pharmaceutical active ingredients, as well as pharmaceutical compositions in this context and their applications, in particular for the purpose of administering or applying prophylactically and/or therapeutically active substances, such as vaccine or vaccination active ingredients, antibiotic active ingredients or the like.

In particular, the present invention relates to a method for producing a preferably lipid-based carrier system, which is in particular in particulate form, preferably in the form of lipid nanoparticles (LNP), and which is in particular introduced or produced in a liquid medium (i.e. in particular a liquid medium at a temperature in the range from 20° C. to 40° C., preferably room temperature or 20° C., and ambient pressure or 1,013.25 hPa, respectively) for forming a composition thereof, wherein the preferably lipid-based carrier system can be loaded or equipped with a pharmaceutical active ingredient. In this context, the present invention also relates to the preferably lipid-based carrier system obtainable by the method according to the invention and to a carrier system as such.

Furthermore, the present invention also relates to a composition which is in particular in the form of a dispersion and which contains the carrier system according to the invention, preferably a lipid-based carrier system, wherein the carrier system is in particular incorporated in a liquid medium or supplied therein. The present invention also relates to a pharmaceutical or medicinal product comprising the carrier system or composition according to the invention, in particular together with at least one pharmaceutically acceptable excipient. In this context, the present invention also relates to the applications or uses of the pharmaceutical or medicinal product according to the invention, for example in the context of training or use as a vaccine or antimicrobial composition.

When administering therapeutic (e.g. antibiotic substances) or prophylactic (e.g. substances acting as a vaccine) active ingredients, there is generally a pharmacological need for the active ingredients to be present in pharm pharmacologically effective amount at the site of action, such as special organs, tissues or cells or cell systems, and this also against the background that corresponding active ingredients are often unstable or sensitive to external influences, such as high or low temperatures or the like. Consequently, with regard to corresponding compositions comprising such active ingredients, appropriate measures are required which, on the one hand, stabilize the active ingredient and, on the other hand, lead to an efficient release or bioavailability of the active ingredient at the site of action, for example a (body) cell, wherein such compositions comprising active ingredients should also as a whole be well tolerated and have a low side-effect profile, and this with good overall manageability, manufacturability and (storage) stability. In particular, it is also necessary in this context that the active ingredients incorporated in a composition, as mentioned above, are further stabilized or protected from external influences, such as high temperatures or UV radiation, so that, for example, a correspondingly broad temperature profile is present.

In view of this, active ingredients are often supplied in suitable carrier systems or coupled with them, as is the case, for example, with micelle-, vesicle- and liposome-based or other encapsulations.

In this regard, active ingredients or compositions based on polynucleotides, such as ribonucleic acids (RNA) (ribonucleic acid), in particular messenger RNA (mRNA), are known in the prior art, which have recently been used in particular as vaccines, in particular with a view to immunization against the SARS-CoV-2 virus (severe acute respiratory syndrome corona virus type 2), which belongs to the family of coronaviruses. In this context, the substance, in the form of a special mRNA, codes for an antigen of the virus, namely with regard to the so-called spike protein of the coronavirus (“spike protein”), wherein this protein is positioned on the surface of the virus and plays a role in cell infection, in particular on the basis of an interaction with special proteins on or in the cell membrane of (body) cells (ACE-2, angiotensin-converting enzyme 2). With regard to the underlying mRNA-based vaccines, the sensitive mRNA is supplied in a carrier system based in particular on lipids or lipid-based particles to protect it and enable it to be absorbed into (body) cells. After being taken up by the (body) cells, the blueprint encoded by the mRNA is read by the (body) cell, as it were, and the spike protein (as an antigen) is subsequently produced and presented on the surface of the (body) cell, so that it can be recognized by immune cells of the body, which in turn activates the immune system and, among other things, antibodies are formed against the spike protein, which acts as an antigen, wherein an “immunological memory” is also evoked, accompanied by a certain protection against subsequent infections.

In principle, nanostructured compositions, such as those based on liposomes or vesicles or lipid (nano)particles as carriers or encapsulations of corresponding active ingredients, such as mRNA in particular, as mentioned above, are suitable.

However, such compositions or systems are generally problematic in terms of producing them, as they can often only be produced with great effort and by drawing on extensive process know-how. In this context, it is often a disadvantage in the state of the art that the corresponding compositions are inhomogeneous, in particular with regard to the size of the underlying liposomes or lipid (nano)particles, wherein a constant product quality over the storage period is also not always satisfactory. In particular, this often results in excessively large particles based on lipids or the like, or excessively large liposomes or excessively large lipid (nano)particles in the composition, often accompanied by inhomogeneous particle size distribution and a high dispersity or large variance, which can be problematic in particular with regard to product quality, storage stability, packaging of the active ingredient and application in the body, as well as associated side effects. The causes of the inhomogeneities that sometimes occur can also be attributed to the fact that it is often difficult in the state of the art to supply or dissolve the hydrophilic or lipophilic main components, which form the basis for the liposomes or lipid (nano)particles, in an optimal way in an aqueous environment. In addition, the components used for the corresponding compositions of the state of the art are often disadvantageous to the particle structures present in the compositions, for example when the compositions are frozen for storage purposes or when they are subsequently thawed.

Furthermore, it is often necessary in the state of the art to supply a high input of energy to form corresponding particulate structures, for example on the basis of liposomes or lipid (nano)particles, in a composition containing the components for forming the particulate structures, for example in the form of ultrasound or the like. However, the high input of energy required for this can lead to the destruction or inactivation of the active ingredient, especially since mRNA, for example, is a substance that is sensitive not only to temperature influences but also to the application of force. It can also be problematic that a correspondingly high input of energy is associated with an excessive increase in temperature of the underlying system or composition, which can further impair the active ingredient, especially since high temperatures are already required in the state of the art for producing the compositions, in particular due to the sometimes poor solubility of the particles. In this context, too, heating and cooling processes or steps are sometimes required, which, in addition to a high energy input, are also associated with complicated process operation and control and expensive process equipment or devices.

Overall, producing the corresponding active ingredient-containing compositions in the state of the art, which comprise lipid (nano)particle structures or vesicles as carriers of the active ingredient, such as mRNA, is relatively complex, wherein no satisfactory homogeneous vesicle structures or sizes are obtained.

Furthermore, the state of the art sometimes requires the components used for the compositions to be used in corresponding premixes or solutions, wherein alcohol in the form of ethanol or chloroform is often used as a solvent, which, however, may have to be removed in a complex manner in the further manufacturing process. The aforementioned substances can also lead to incompatibilities, in particular with regard to the active ingredients to be used, which can limit the number of active ingredient types or types to be used or affect their effectiveness.

Due to the process operation for producing such substances, which is state of the art, the compositions often also result in undesirably large multilayer vesicles (MLVs), which are subsequently converted or processed into smaller and in particular unilamellar vesicles using high-pressure homogenizers or subsequently converted or processed into smaller and in particular unilamellar vesicles, which is equally complex in terms of process engineering and leads to an additional physical stress on the underlying composition or the active ingredient system.

In particular, the state of the art requires that the ethanol or chloroform used to dissolve the components, in particular in corresponding premixes, be removed from the resulting composition, for example using evaporation or extraction or the like.

In the methods for producing corresponding vesicle-based or lipid (nano)particle-based compositions containing active ingredients that have been known to date, there are often certain fluctuations in the product quality overall, in particular with regard to the particle sizes and particle size distributions of the underlying vesicles or lipid (nano)particles, wherein particles in the high micrometer range often result or are present. Sometimes there can also be significant fluctuations, in particular batch-to-batch fluctuations, in the quality of the production.

In general, the structure of the underlying particles, in particular in the form of vesicles or lipid (nano)particles, is guaranteed on the basis of special lipids, which can also occur in biological membranes. In addition, cholesterol is often used. In terms of their composition and structure, the underlying vesicles or lipid (nano)particles generally serve the same purpose as the so-called liposomes, which have been used for a long time as carriers for drugs or active ingredients in the field of medicine. In general, in particular with regard to the use of messenger RNA (mRNA), in particular as a vaccine, the underlying particulate structure or the vesicle or lipid (nano)particles enable the active ingredient to be transported to the (body) cells and absorbed, wherein the active ingredient can then exert its effect.

WO 2018/078053 A1, EP 3 532 094 A1, which belongs to the same patent family, US 2020/0163878 A1 and US 2021/251 898 A1 relates to mRNA comprising lipid nanoparticles and their medical uses, as well as a method for producing the lipid nanoparticle, wherein some of the components are first dissolved in ethanol and the ethanol is then removed, in particular using dialysis or diafiltration.

Overall, there is therefore a great need in the state of the art for corresponding carrier-based compositions based on particulate lipid-based structures, in particular lipid nanoparticles, wherein these systems are to be suitable for the uptake or administration of corresponding active ingredients, and indeed also the great potential of drugs based on this with regard to the prophylactic or therapeutic treatment of underlying diseases or with regard to their use as a vaccine or the like.

Against this background, the object of the present invention is therefore to provide an efficient concept for the provision of a preferably lipid-based carrier system present in particulate form, which is suitable for equipping or loading with a pharmaceutical active ingredient, wherein the previously described disadvantages of the state of the art are to be at least largely avoided or at least mitigated.

In particular, one object of the present invention is to provide a method for producing such a preferably lipid-based carrier system or such carrier systems as such, which, in particular with regard to the formation of the particles in the form of lipid nanoparticles (LNP) particles in the form of lipid nanoparticles (LNP) to particles with small sizes, in particular in the nanometer range, with a homogeneous particle size distribution at the same time, wherein in this regard a low dispersity or low variance should also be present.

Likewise, a further object of the present invention is to provide a method for producing preferably lipid-based carrier systems or such carrier systems as such, wherein the carrier systems provided are excellently for equipping or loading with pharmaceutical active ingredients, and indeed also with regard to a multitude of different types of active ingredients, so that a universal carrier system is to be provided according to the invention.

In this context, a further object of the present invention is also to be seen in the fact that a method or a carrier system as such is provided in accordance with the invention, wherein the carrier system produced in accordance with the invention is to be present in compositions based thereon in accordance with the invention wherein the compositions comprising the carrier system therein are to comprise an optimal application or use in the context of the prophylactic or therapeutic treatment of diseases on the basis of corresponding drugs or medications, while at the same time having a high level of compatibility and a high (storage) stability.

In this context, a further object of the present invention is to ensure a high compatibility of the carrier system or compositions thereof with the active ingredients to be used.

In addition, the invention is also intended to provide in particular such a method for producing corresponding preferably lipid-based carrier systems or carrier systems according to the invention as such, which are preferably lipid-based, wherein the underlying preparation process is to be well controllable and/or controllable overall and to lead to consistently high product qualities, also with regard to ensuring the formation of defined particle structures, in particular in the form of lipid nanoparticles. In addition, it should also be ensured that the underlying method for producing the preferably lipid-based carrier system can be used on a total of less complex devices and with as few method steps as possible.

### BRIEF SUMMARY OF THE INVENTION

In this context, a further object of the present invention is to provide improved drugs or medications based on the carrier systems, in particular lipid-based carrier systems.

To solve the object described, the present invention—in accordance with a first aspect of the present invention—proposes a method for producing a preferably lipid-based carrier system, preferably in particulate form, preferably in the form of lipid nanoparticles (LNP), in particular for a pharmaceutical active ingredient; and/or for producing a preferably lipid-based carrier system loaded or equipped with a pharmaceutical active ingredient, preferably in particulate form, preferably in the form of lipid nanoparticles (LNP); respectively advantageous further developments and configurations of this aspect of the invention are described relating to the method according to the invention.

Furthermore, the present invention relates—according to a second aspect of the present invention—to a preferably lipid-based carrier system obtainable by the method according to the invention, which is in particular in particulate form, preferably in the form of lipid nanoparticles, and which is in particular loaded or equipped with a pharmaceutical active ingredient or a corresponding, preferably lipid-based carrier system as such; further, in particular advantageous, further developments and configurations of this aspect of the invention are the subject-matter relating to the preferably lipid-based carrier system according to the invention.

In addition, the present invention—according to a third aspect of the present invention—also relates to a composition which contains the lipid-based carrier system according to the invention in a liquid medium; in each case advantageous further developments and configurations of this aspect of the invention are the subject-matter of the disclosure relating to the composition.

A further subject-matter of the present invention—according to a fourth aspect of the present invention—is also the pharmaceutical composition according to the invention, in particular for use in the prophylactic and/or therapeutic treatment of diseases of the human or animal body, which contains the carrier system according to the invention, in particular a lipid-based carrier system, which is in particular loaded or equipped with a pharmaceutical active ingredient or a composition thereof, in particular together with at least one pharmaceutical excipient described herein and relating to the pharmaceutical agent or medicine according to the invention; further advantageous developments and configurations of this aspect of the invention are described relating to the pharmaceutical agent or medicine according to the invention.

In this context, the present invention relates in particular to the use of the pharmaceutical agent or carrier system or composition according to the invention for the prophylactic and/or therapeutic treatment of diseases of the human and/or animal body or for producing a pharmaceutical agent for the prophylactic and/or therapeutic treatment of diseases of the human or animal body, as defined herein. In this context, the present invention also relates to a method for the prophylactic or therapeutic treatment of diseases of the human or animal body. The present invention also relates to the use of at least one process parameter for controlling, in particular for process control, the method described herein.

In the context of the following explanations, it goes without saying that configurations, embodiments, advantages and the like, which are only mentioned below for the purpose of avoiding repetition, naturally also apply to the other aspects of the invention, without this requiring separate mention.

In the case of all the relative or percentage weight-related data, in particular quantity data, mentioned below, it should also be noted that, within the context of the present invention, they are to be selected by the skilled person in such a way that, in total always add up to 100% or 100 wt. %, respectively, including all components or ingredients, in particular as defined below; however, this is self-evident for the skilled person.

Furthermore, it is understood that the skilled person may deviate from the following concentration, weight, amount and range specifications, depending on the application or individual case, without departing from the scope of the present invention.

Furthermore, it is understood that all the values, parameters or the like mentioned below can be determined or defined in principle by means of standardized or explicitly stated determination procedures or, failing that, by means of determination or measurement methods that are in themselves familiar to the expert in the field.

With this in mind, the present invention will now be explained in detail below.

### BRIEF DESCRIPTION OF THE DRAWINGS

FIG. 1A a schematic and illustrative representation of the structure or shape of a particle of the carrier system according to the invention in the form of a disc-shaped lipid nanoparticle 1 (also known as a disc lipid nanoparticle (disc-LNP), disc vesicle or disk-lamella); the disk-shaped lipid nanoparticle comprises in particular a cylindrical or disk-shaped geometry with a peripheral region or a lateral surface 1a and in particular circular or in particular circularly shaped base surfaces 1b

FIG. 1B a schematic and illustrative representation of a disc-shaped lipid nanoparticle 1, according to which the surface area or the edge region has non-ionic amphiphilic or surfactant substances, in particular PEGylated substances or PEGylated surfactants 2 (see substance (ii)), while the shell surfaces comprise mainly phospholipids (see substance (i)); in this context, the underlying HLB value according to Griffin or the respective critical packing parameter is also relevant for the arrangement of the substances in the disc-shaped lipid nanoparticle, according to which substances with a relatively high Griffin HLB value or a relatively low critical packing parameter are primarily arranged in the peripheral area or in the area of the shell surface, while substances with a relatively low Griffin HLB value or a relatively high critical packing parameter are primarily arranged in the area of the base surfaces;

FIG. 1C a schematic and illustrative representation of a disc-shaped lipid nanoparticle 1 of the carrier system according to the invention, which in the peripheral region likewise comprises the substances 2 mentioned above in FIG. 1B and according to which the lipid nanoparticle is multilayer, in particular on the basis of further phospholipids 4, which are arranged in particular parallel to the base surfaces of the underlying disc-shaped structure, and further substances 3, which are arranged in the shell region and in particular can interact with the substance 2; the particles according to FIG. 1A to FIG. 1C are shown in particular for purposes of illustration as a perspective sectional view, wherein the respective circular representations illustrate the hydrophilic regions and the respective line representations illustrate the lipophilic regions of the underlying molecules;

FIG. 2 a schematic and illustrative representation of the formation of the carrier system particles according to the invention on the basis of the respective basic structures in the form of a disc-shaped lipid nanoparticle 1; FIG. 2 also shows further, although less preferably, configurations of the particles, namely in the form of a vesicle 5, a spherical micelle 6 and a worm-like micelle 7; the particles 1, 5, 6 and 7 are in particular illustrated in the form of a sectional representation, wherein the respective circular representations and the respective line representations in particular represent the lipophilic region of the underlying molecules;

FIG. 3 a schematic and illustrative representation of another particulate form or particle 8 with components based on PEGylated substances 9, phospholipids 10, cholesterol 11 and active ingredient 12;

FIG. 4 a graphical representation in the form of a diagram to illustrate the relationship between the packaging parameter Pₖᵣ on the one hand and the HLB value according to Griffin on the other;

FIG. 5 a schematic representation of a method of operation according to the invention based on respective batches or a divided supply of the ingredients or components a1 to c1 in respectively assigned receiving or processing devices A1 to C1 in the context of a continuous or cascaded and/or consecutive process operation for obtaining the composition;

FIG. 6A a schematic representation of a method according to the invention with an exemplary division of the substances of the carrier system or the components for the composition to be produced for the purpose of forming corresponding batches or starting from respective storage devices a2 to d2 and with processing in corresponding receiving devices (A2 to D2) in the context of a continuous and in particular cascaded or consecutive process operation with successive combining of the batches according to an embodiment of the invention;

FIG. 6B a schematic representation based on FIG. 6A according to a further embodiment of the invention with an alternative distribution of the substances or components; the reference signs a3 to d3 correspond to a2 to d2 according to FIG. 6A, wherein the same applies to A3 to D3 and A2 to D3;

FIG. 7 a cryo-scanning electron microscope image of an invention-composition with the invention-carrier system based on particles in the form of disk-shaped lipid nanoparticles; the average particle diameter is 150 nm±70 nm; polysorbate 20 and retinol are used as amphiphilic main components;

FIG. 8 a graphical representation in the form of a diagram to illustrate the relationship between the HLB value according to Griffin on the one hand and the Log P value on the other hand (in semi-logarithmic coordinates) as shown in the context of the invention.

### DETAILED DESCRIPTION OF THE INVENTION

The subject-matter of the present invention—according to a first aspect of the present invention—is thus a method for producing a preferably lipid-based carrier system, preferably in particulate form, preferably in the form of lipid nanoparticles (LNP), in particular for a pharmaceutical active ingredient; and/or for producing a preferably lipid-based carrier system loaded and/or equipped with a pharmaceutical active ingredient, preferably in particulate form, preferably in the form of lipid nanoparticles (LNP),
wherein the components of the carrier system are brought into contact with each other and/or are brought into interaction with each other, in particular are brought into physical and/or chemical interaction with each other, in such a way that an agglomeration, in particular an aggregation, of the components is effected to form the carrier system, preferably in particulate form, preferably in the form of lipid nanoparticles (LNP),
wherein the components of the carrier system comprise the following substances:
   (i) at least one phospholipid, preferably phosphatidylcholine (lecithin);
      in particular wherein the phospholipid has a Griffin HLB value of at most 11, in particular at most 8, and/or in particular wherein the phospholipid has a Log P value of at least 0.55, in particular at least 0.80;
   (ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferably selected from the group of PEGylated fatty and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids, as well as their combinations and mixtures, even more preferably selected from PEGylated fatty acids and their salts and esters;
      in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
   (iii) optionally at least one steroid and/or secosteroid, in particular at least one sterol (sterol), preferably selected from the group of zoosterols, phytosterols and mycosterols, as well as combinations and mixtures thereof, preferably cholesterol and/or phytosterol, and/or in particular at least one secosterol, preferably at least one calciferol (vitamin D);
      in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
   (iv) optionally at least one cationic lipid, in particular an amine-based cationic lipid, preferably a cationic lipid based on a tertiary amine;
      in particular wherein the cationic lipid comprises an HLB value according to Griffin of at most 4 and/or in particular wherein the cationic lipid comprises a Log P value of at least 1.76;
   (v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or tri-fatty acid ester of glycerol (mono-, di- or tri-fatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
      in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
in particular wherein the method, in particular producing the carrier system or the carrier system loaded and/or equipped with the pharmaceutical active ingredient, is carried out in a liquid medium, in particular in a liquid medium at a temperature in the range from 20° C. to 40° C., preferably at room temperature (20° C.) and ambient pressure (1,013.25 hPa), preferably in the form of a dispersion medium (dispersing agent), and after formation of the carrier system, a preferably disperse composition, in particular a dispersion, which comprises the carrier system or the carrier system loaded and/or equipped with the pharmaceutical active ingredient and/or the carrier system loaded and/or equipped with the pharmaceutical active ingredient and the liquid medium, or a dispersion of the carrier system or the carrier system loaded and/or equipped with a pharmaceutical active ingredient in the liquid medium results after formation of the carrier system;
wherein the agglomeration, in particular the aggregation, of the constituents for forming the carrier system is carried out with the input of energy, preferably the input of mixing and/or stirring energy, in particular into the liquid medium comprising at least some of the constituents and/or substances of the carrier system and/or into a mixture comprising at least some of the constituents and/or substances of the carrier system, wherein the energy introduced, in particular mixing and/or stirring energy, is set in such a way that an at least substantially laminar flow is present, in particular in the liquid medium comprising at least some of the components and/or substances of the carrier system and/or in the mixture comprising at least some of the components and/or substances of the carrier system, in particular by at least substantially completely avoiding the formation of a non-laminar and/or turbulent flow and/or in particular by at least substantially completely avoiding the formation of non-laminar and/or turbulent flow components; and/or
wherein a, in particular, continuous method and/or process control (production control), in particular online and/or inline process control, is carried out, in particular for monitoring the method status and/or method progress, preferably for monitoring and/or setting the formation of the particles of the carrier system, in particular lipid nanoparticles (LNP), and/or in particular for monitoring and/or setting process parameters, such as the input of energy, preferably the input of mixing and/or stirring energy, the input and/or removal of thermal energy, flow behavior, mass throughput, volume flows or the like; wherein the method and/or process control is carried out via acquiring at least one process parameter, in particular at least two process parameters, wherein the process parameter is selected from the group of temperature, conductivity, viscosity and pH value, in particular of the liquid medium, preferably together with the components and/or substances introduced therein, and/or of the composition.

According to the invention, a special method for producing preferably lipid-based carrier systems is provided, wherein, within the scope of the present invention, special components of the carrier system are brought into contact or interaction in a special selection and coordination for forming defined particulate forms, in particular for forming lipid nanoparticles (LNP). According to the invention, this results in a corresponding carrier system in particulate form with defined properties. In particular, the carrier system is loaded or equipped or can be loaded or equipped with at least one pharmaceutical active ingredient. In particular, the carrier system can thus comprise at least one pharmaceutical active ingredient, in particular as a (further) component or substance of the carrier system (see also the following configurations).

The applicant has surprisingly found out that the so-called HLB value according to Griffin, which is assigned to the respective components or substances, is of significant importance, in particular with regard to the formation of homogeneous and nanoparticulate structures as well as in the form of lipid nanoparticles with low dispersity or small variance, wherein, on the basis of the special process operation, (storage-) stable compositions can also be provided in this regard and the carrier systems in question can be loaded or equipped with a variety of different pharmaceutical active ingredients, as detailed below.

In the context of the present invention, it is completely surprising that the use of a substance (i) in the form of a phospholipid, which in particular comprises an HLB value according to Griffin of at most 11, in particular at most 8, in combination with the substance (ii), namely in the form of non-ionic amphiphilic or tenside substances, such as tenside PEGylated substances, in corresponding compositions or fluid media, leads to the formation of particularly defined particulate forms, preferably in the form of lipid nanoparticles. On this basis too, homogeneous particulate forms can be formed or provided in terms of their size.

As regards the HLB value according to Griffin cited in the invention, this in particular describes the hydrophilic and lipophilic portion of corresponding substances. The HLB value according to Griffin can in particular be calculated according to the formula 20×M_{hydrophil}/Mₜₒₜₐₗ, wherein M_{hydrophil} relates to the molecular weight of the hydrophilic portion or the head group of the underlying substance or molecule and wherein Mₜₒₜₐₗ relates to the molecular weight or molar mass of the entire substance or molecule. The factor 20 represents a defined scaling factor. On the basis of the HLB value according to Griffin, a scale from 0 to 20 results, wherein an HLB value according to Griffin of 1 describes a lipophilic substance overall, while an HLB value according to Griffin of 20 describes a hydrophilic substance overall. Alternatively, the HLB value according to Griffin can also be calculated according to the formula HLB=20×(1−Mₗᵢₚₒₚₕᵢₗ/Mₜₒₜₐₗ), wherein Mlipophil refers to the lipophilic portion of a substance or molecule.

The lipophilic or hydrophilic properties of the substances used in accordance with the invention can thus be characterized using the HLB value according to Griffin, wherein the substances used as components of the carrier system in accordance with the invention, as defined herein, lead to the formation of carrier systems in particulate form, in particular in the form of lipid nanoparticles, with overall improved properties. In the course of this, the applicant also surprisingly found that, in the context of the method according to the invention, with regard to the carrier system according to the invention and the particles on which it is based, in particular lipid nanoparticles, particularly defined properties result if the substances used for the carrier system, in particular amphiphilic substances, in their entirety or on the basis of special combinations of the underlying substances, comprise an average HLB value according to Griffin in a defined range, in particular in a range from 3 to 8. This is all the more surprising as such a value is usually assigned to W/O emulsifiers (water-in-oil emulsifiers).

For further information on the Griffin HLB value, please refer in particular to Griffin “W. C. Classification of Surface Active Agent by HLB”, J. SOC. Cosmet. Chem. 1, 1949. In addition, please refer to “Heusch, an experimental method for determining the HLB value of surfactants”, Cholloid-Zeitschrift und Zeitschrift für Polymere, Vol. 236, Issue 1.

For zwitterionic substances and phospholipids, the HLB value according to Griffin refers in particular to that which is present at the isoelectric point.

The applicant has also surprisingly found that the so-called Log P value, which is assigned to the respective components or substances, is also of further significance, in particular with regard to the formation of homogeneous and nanoparticulate structures based on defined carrier systems.

The Log P value (also known as log 10 P or lg P) is the common logarithm of the partition coefficient P for a particular substance. In general, the P value can be determined as the ratio of the concentrations of the substance in an n-octanol phase and a water phase. The Log P value is a measure of the lipophilicity of a substance or a pharmaceutical active ingredient. In particular, the Log P value can also be used to estimate permeability, absorption and bioavailability. In general, the lipophilicity of a substance increases with increasing Log P value. Conversely, small, in particular negative, Log P values indicate a high degree of hydrophilicity.

As mentioned above, the Log P value is the common logarithm of the partition coefficient P or the P value. The P value is a partition coefficient that is determined using a biphasic system with the two solvents n-octanol and water. The concentrations in the two phases are determined. To calculate the P-value, the concentration of the substance in n-octanol is divided by the concentration of the substance in water: P=c(n-octanol)/c(water). The Log P-value is the common logarithm of P, i.e. log P=log(c(n-octanol)/c(water)).

The Log P value is thus a substance-specific parameter that can be determined on the basis of the approach described above. The relevant approach and determination are well known to experts in the field. In addition, the corresponding Log P values are also listed in standard works.

Furthermore, the Log P values can also be determined in particular in a computational or computer-based way, in particular using the application or tool X Log P3. In particular, the determination of the Log P values can thus be effected in the present case using the X Log P3 method. Reference can also be made to Cheng, T. et al., “Computation of Octanol-Water Partition Coefficients by Guiding an Additive Model with Knowledge”, J. Chem. Inf. Model. 2007, 47, 2140-2148. In particular, very precise values can be obtained on the basis of the calculation-based or computer-aided determination.

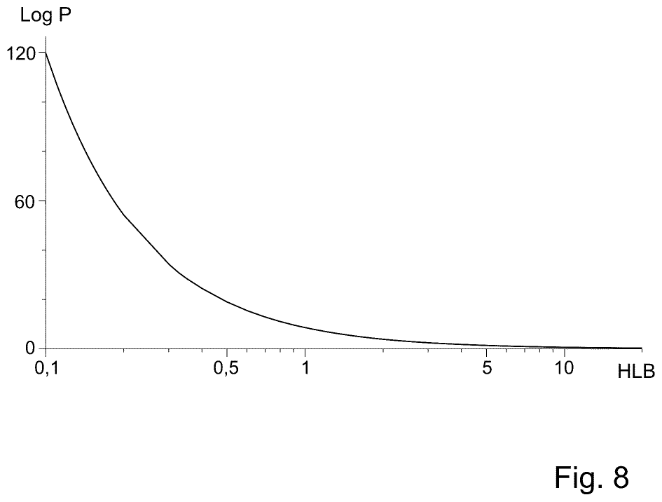

On the basis of the Log P value, a further characterization of the components or substances used is available within the scope of the present invention. In this context, the applicant has surprisingly found a functional relationship between Log P values on the one hand and HLB values on the other hand for the components or substances used or carrier systems according to the invention. In this context, reference can also be made to FIG. 8 and the following explanations.

In particular, the applicant was able to demonstrate a functional relationship based on the formula HLB=6.5469·Log P^{-0.874} (see also FIG. 8, which graphically illustrates the functional relationship based on the aforementioned formula (with the corresponding formula conversion)).

In the context of the present invention, the Log P values can thus be used with regard to the HLB values according to Griffin as an alternative and/or cumulative measure for describing the lipophilicity or hydrophilicity of the substances or components used. On this basis, a supplementary and further characterization of the carrier systems obtained according to the invention can also be performed. On the basis of or by using the corresponding Log P values, a further characterization and description of the lipophilicity of the underlying components can thus be carried out. In particular, the resulting properties with regard to passive diffusion, absorption and bioavailability can also be further characterized on this basis.

Overall, the use of the Log P values also provides a characterization of the underlying components, substances and carrier systems obtained that is complementary to and more detailed than the HLB values according to Griffin. In particular, in this context, n-octanol is used as the distribution system for determining the Log P value, as mentioned above, in comparison to water. In this context, n-octanol, as a long-chain alcohol, is also generally used as a model for the cell membrane, which is mainly composed of amphiphilic phospholipids. For this reason, too, the Log P value is particularly suitable for characterizing the components used and the resulting carrier systems according to the invention, also with regard to their bioavailability and interaction with active ingredients or the like.

The targeted and purposeful use of special components or substances with a defined HLB value according to Griffin on the one hand and a defined Log P value on the other hand, and the coordination of these values, can be used to provide optimized carrier systems overall, also with regard to their formation or behavior in the liquid medium, also taking into account, for example, polyol as a further component of the underlying liquid medium. On this basis, defined, stable and customized carrier systems can be developed.

As far as the HLB value according to Griffin and the Log P value are concerned, these can be determined in each case in particular under standard conditions (i.e. at room temperature (20° C.) and ambient pressure (1,013.25 hPa)).

In addition, the input of energy, in particular mixing or stirring energy, into the underlying system in a special way, as provided for in the invention, leads to the formation of the carrier system according to the invention or the composition according to the invention, under providing a substantially laminar flow and avoiding the formation of a non-laminar or turbulent flow, in a completely surprising manner, to further improved carrier systems or compositions according to the invention. Because the formation of a laminar flow results in—without wishing to limit or refer to this topic—a particularly defined input of energy, which does not adversely affect the underlying self-aggregation of the components to form the carrier system. Rather, the input of energy is particularly gentle on the basis of the inventive measure, which leads to the formation of defined particle systems with a narrow particle size distribution while at the same time having a defined particle size and defined physical formation (e.g. in the form of disc LNPs). In particular, the occurrence of defect structures or the like is effectively prevented or minimized.

Furthermore, the process control provided for in a targeted manner within the scope of the invention, while acquiring special process parameters, enables a further optimized process performance. In particular, on this basis, corresponding process parameters can be adapted or adjusted even during the process operation, so that on this basis, too, there are overall uniform or specifically controlled process conditions for the formation of optimal carrier systems or compositions related thereto according to the invention.

In this context, the measures of the invention for the controlled input of energy, with the formation of a laminar flow, and the special process control complement each other in a synergistic manner that goes beyond the sum of the individual measures. Consequently, optimized or customized carrier systems with a defined particle formation and compositions based on them can also be provided in accordance with the invention.

With regard to acquiring special process parameters, for example, a sudden (relative) change in a process parameter (e.g. conductivity) may indicate an unwanted change in the structural composition of the underlying system or medium with the relevant components. If the process parameters change accordingly, the relevant process parameters can then be changed or adjusted to ensure an optimal manufacturing process.

On the basis of the concept according to the invention, a preferably lipid-based carrier system is provided which can be equipped or loaded with active ingredients in a targeted and purposeful manner. By selecting and coordinating the respective substances, customized or optimized carrier systems can also be provided in this regard in order to enable optimal equipping with the active ingredient. As mentioned above, the systems according to the invention are characterized in particular by small particle sizes, wherein in particular special lipid nanoparticles (LNP) are formed or present, in particular in the form of disc-shaped (slice-shaped) lipid nanoparticles, as will be explained below. The particles, in particular lipid nanoparticles (LNP), are relatively small in size or particle diameter, and this with a homogeneous particle size distribution or lower dispersity and/or variance, so that the particles of the carrier system according to the invention, in particular those present in a liquid medium, are highly homogeneous. This is accompanied by defined properties of the underlying compositions, including their (storage) stability and their defined release of active substances or active ingredients at the site of action (e.g. (body) cells). In addition, an optimal loading or equipping of the carrier system with active ingredients as relevant components can be realized.

On this basis, too, a universal carrier system is provided overall within the scope of the present invention, which is suitable for a large number of active ingredient types or types and can be customized individually in this regard.

In addition, the method according to the invention is based on a small number of process steps, wherein the method according to the invention can be carried out using relatively simply constructed devices and with excellent process control, which not least leads to consistent product qualities and an applicability of the method according to the invention. In this regard, a high degree of automation can also be realized. In addition, the method according to the invention is characterized in that high throughputs can be achieved, so that a cost-optimized or overall economical method is provided.

In particular, the method according to the invention makes it possible to dispense with the use of substances that are sometimes problematic or physiologically questionable, such as ethanol and chloroform, so that in this respect, complex removal steps are also no longer necessary, which is beneficial for product quality and economy. In particular, this also improves the compatibility of compositions resulting from the method with regard to their use as a drug or medication.

As regards the agglomeration or aggregation of the components of the carrier system based on the substances mentioned in general, this is based—without wishing to limit or rely on this theory—for example or in particular on entropy-driven processes. In addition, the aggregation or agglomeration can be caused by van der Waals forces, intermolecular interactions, chemical bonds or the like. As will be explained in detail below, in the context of the present invention, the agglomeration or aggregation is in particular caused or induced by the input of energy, in particular mixing and/or stirring energy, into the liquid medium in which the components or substances are supplied, wherein the resulting particle sizes or particle diameters can also be set in a targeted manner on this basis. This is effected in particular with regard to the dispersity or variance of the underlying particles of the lipid-based carrier system according to the invention in this context.

As mentioned above, the respective HLB value according to Griffin is of importance with regard to the formation of the particles, in particular lipid nanoparticles (LNP), of the carrier system according to the invention, in particular the lipid-based carrier system. By specifically selecting and matching the components with regard to their respective HLB value according to Griffin, the resulting particles, in particular lipid nanoparticles (LNP), can be further set in terms of their structure, shape and size, or the relevant properties can be specifically predetermined. The selection and coordination of the substances in dependence on their HLB values according to Griffin also leads to an improved processability and/or manageability and/or handling of the substances within the scope of the process operation according to the invention, in particular with regard to the incorporation into the underlying fluid medium for the formation of the composition with the carrier system present therein on the basis of the particles, which will be dealt with in detail below.

With regard to the input of energy as envisaged by the invention, the following procedure in particular has proved advantageous: The amount of energy introduced, in particular the amount of mixing and/or stirring energy introduced, can be set in such a way that a Reynolds number Re, in particular a Reynolds number Re related to a stirrer flow, of at most 2,000, in particular at most 1,500, preferably at most 1,000, and more preferably at most 500, is present.

With regard to the process operation according to the invention, it has proved to be particularly advantageous if the substances mentioned above each comprise the following properties: In particular, the substance (i) can have an HLB value according to Griffin in the range from 0.5 to 8, preferably in the range from 2 to 7, preferably in the range from 3 to 6, and in particular the substance (i) comprise a Log P value in the range from 0.80 to 18.97, preferably in the range from 0.93 to 3.88, preferably in the range from 1.11 to 2.44.
In addition, the substance (ii) may comprise an HLB value according to Griffin in the range of 13 to 19, in particular in the range of 13 to 17.9, preferably in the range of 13.5 to 17.5, preferably in the range of 14 to 17, or substance (ii) may also comprise a Log P value in the range from 0.30 to 0.46, in particular in the range from 0.32 to 0.46, preferably in the range from 0.33 to 0.44, preferably in the range from 0.34 to 0.42.
Furthermore, the substance (iii) may comprise an HLB value according to Griffin in the range of 0.5 to 1.5, preferably in the range of 0.6 to 1.4, preferably in the range of 0.7 to 1.3, or substance (iii) may further comprise a Log P value in the range of 5.40 to 18.97, preferably in the range of 5.84 to 15.40, preferably in the range of 6.36 to 12.91.
The substance (iv) may comprise an HLB value according to Griffin in the range of 1.5 to 3.5, preferably in the range of 2 to 3, or the substance (iv) may comprise a Log P value in the range of 2.05 to 5.40, preferably in the range of 2.44 to 3.88.
The substance (v) may comprise an HLB value according to Griffin in the range of 1 to 8, preferably in the range of 1.25 to 7, preferably in the range of 1.5 to 6.5, or the substance (v) may comprise a Log P value in the range from 0.80 to 8.58, preferably in the range from 0.93 to 6.65, preferably in the range from 1.01 to 5.40.

The selection of the substances according to the invention with regard to the respective underlying HLB value according to Griffin makes it possible in particular to control the specific formation or shaping of the particles of the preferably lipid-based carrier system according to the invention, wherein, for example, lipid nanoparticles (LNP) in the form of disc-shaped (disk-shaped) lipid nanoparticles can be obtained by the special selection and coordination of the substances on the basis of the HLB value according to Griffin, which comprise particularly advantageous properties.

In particular, the method can behave in such a way that it is characterized by at least one of the following features and/or measures (1a) to (5a), in particular by a combination of at least two, preferably at least three, preferably at least four, particularly preferred all five, of the following features and/or measures (1a) to (5a):
(1a) the substance (i) has a Griffin HLB value of at most 11, in particular at most 8, in particular in the range from 0.5 to 8, preferably in the range from 2 to 7, preferably in the range from 3 to 6, and/or substance (i) comprises a Log P value of at least 0.55, in particular at least 0.80, in particular in the range from 0.80 to 18.97, preferably in the range from 0.93 to 3.88, preferably in the range from 1.11 to 2.44;
(2a) the substance (ii) comprises an HLB value according to Griffin of at least 13, in particular in the range from 13 to 19, preferably in the range from 13 to 17.9, preferably in the range from 13.5 to 17.5, particularly preferred in the range from 14 to 17, and/or substance (ii) comprises a Log P value of at most 0.46, in particular in the range from 0.32 to 0.46, preferably in the range from 0.33 to 0.44, preferably in the range from 0.34 to 0.42;
(3a) the substance (iii) comprises an HLB value according to Griffin of at most 1.5, in particular in the range from 0.5 to 1.5, preferably in the range from 0.6 to 1.4, preferably in the range from 0.7 to 1.3, and/or the substance (iii) comprises a Log P value of at least 5.40, in particular in the range from 5.40 to 18.97, preferably in the range from 5.84 to 15.40, preferably in the range from 6.36 to 12.91;
(4a) the substance (iv) comprises an HLB value according to Griffin of at most 4, in particular in the range from 1 to 4, preferably in the range from 1.5 to 3.5, preferably in the range from 2 to 3, and/or the substance (iv) comprises a Log P value of at least 1.76, in particular in the range from 1.76 to 8.58, preferably in the range from 2.05 to 5.40, preferably in the range from 2.44 to 3.88;
(5a) the substance (v) comprises an HLB value according to Griffin of at most 8, in particular in the range from 1 to 8, preferably in the range from 1.25 to 7, preferably in the range from 1.5 to 6.5, and/or the substance (v) comprises a Log P value of at least 0.80, in particular in the range from 0.80 to 8.58, preferably in the range from 0.93 to 6.65, preferably in the range from 1.01 to 5.40.

In this context, the present invention also relates to a Method for producing a preferably lipid-based carrier system, preferably in particulate form, preferably in the form of lipid nanoparticles (LNP), in particular for a pharmaceutical active ingredient; and/or for producing a carrier system loaded and/or equipped with a pharmaceutical loaded and/or equipped with a pharmaceutical active ingredient, preferably a lipid-based carrier system, preferably in particulate form, preferably in the form of lipid nanoparticles (LNP), in particular a process according to one of the preceding claims,
wherein the components of the carrier system are brought into contact with one another and/or are brought into interaction with one another, in particular are brought into physical and/or chemical interaction with one another, in such a way that an agglomeration, in particular aggregation, of the components to form the carrier system, preferably in particulate form, preferably in the form of lipid nanoparticles (LNP), is effected,
wherein the components of the carrier system comprise the following substances:
   (i) at least one phospholipid, preferably phosphatidylcholine;
   (ii) at least one non-ionic amphiphilic and/or surfactant substance, in particular selected from the group of polyglycerol esters, alkyl polyglycosides, amphiphilic and/or surfactant PEGylated (polyoxyethylated) substances and mixtures or combinations thereof; preferably selected from surfactant PEGylated substances, preferably selected from the group of PEGylated lipids, particularly preferably selected from the group of PEGylated fatty and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids, as well as their combinations and mixtures, even more preferably selected from PEGylated fatty acids and their salts and esters;
   (iii) optionally at least one steroid and/or secosteroid, in particular at least one sterol (sterol), preferably selected from the group of zoosterols, phytosterols and mycosterols, as well as combinations and mixtures thereof, preferably cholesterol and/or phytosterol, and/or in particular at least one secosterol, preferably at least one calciferol (vitamin D);
   (iv) optionally at least one cationic lipid, in particular an amine-based cationic lipid, preferably a cationic lipid based on a tertiary amine;
   (v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or tri-fatty acid ester of glycerol (mono-, di- or tri-fatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
wherein the method is characterized by at least one of the following features and/or measures (1a) to (5a), in particular by a combination of at least two, preferably at least three, preferably at least four, particularly preferably all five, of the following features and/or measures (1a) to (5a):
   (1a) the substance (i) comprises an HLB value according to Griffin of at most 11, in particular at most 8, in particular in the range from 0.5 to 8, preferably in the range from 2 to 7, preferably in the range from 3 to 6, and/or the substance (i) comprises a Log P value of at least 0.55, in particular at least 0.80, in particular in the range from 0.80 to 18.97, preferably in the range from 0.93 to 3.88, preferably in the range from 1.11 to 2.44;
   (2a) the substance (ii) comprises an HLB value according to Griffin of at least 13, in particular in the range from 13 to 19, preferably in the range from 13 to 17.9, preferably in the range from 13.5 to 17.5, particularly preferred in the range from 14 to 1 7, and/or the substance (ii) comprises a Log P value of at most 0.46, in particular in the range from 0.32 to 0.46, preferably in the range from 0.33 to 0.44, preferably in the range from 0.34 to 0.42;
   (3a) the substance (iii) comprises an HLB value according to Griffin of at most 1.5, in particular in the range from 0.5 to 1.5, preferably in the range from 0.6 to 1.4, preferably in the range from 0.7 to 1.3, and/or the substance (iii) comprises a Log P value of at least 5.40, in particular in the range from 5.40 to 18.97, preferably in the range from 5.84 to 15.40, preferably in the range from 6.36 to 12.91;
   (4a) the substance (iv) comprises an HLB value according to Griffin of at most 4, in particular in the range from 1 to 4, preferably in the range from 1.5 to 3.5, preferably in the range from 2 to 3, and/or the substance (iv) comprises a Log P value of at least 1.76, in particular in the range from 1.76 to 8.58, preferably in the range from 2.05 to 5.40, preferably in the range from 2.44 to 3.88;
   (5a) the substance (v) comprises an HLB value according to Griffin of at most 8, in particular in the range from 1 to 8, preferably in the range from 1.25 to 7, preferably in the range from 1.5 to 6.5, and/or the substance (v) comprises a Log P value of at least 0.80, in particular in the range from 0.80 to 8.58, preferably in the range from 0.93 to 6.65, preferably in the range from 1.01 to 5.40;
in particular wherein the method, in particular producing the carrier system or the carrier system loaded and/or equipped with the pharmaceutical active ingredient, is carried out in a liquid medium, in particular in a liquid medium at a temperature in the range from 20° C. to 40° C., preferably at room temperature (20° C.) and ambient pressure (1,013.25 hPa), preferably in the form of a dispersion medium (dispersing agent), and after formation of the carrier system, a preferably disperse composition, in particular a dispersion, which comprises the carrier system or the carrier system loaded and/or equipped with the pharmaceutical active ingredient and/or the carrier system loaded and/or equipped with a pharmaceutical active ingredient and the liquid medium, or a dispersion of the carrier system or the carrier system loaded and/or equipped with a pharmaceutical active ingredient in the liquid medium results;
wherein the agglomeration, in particular the aggregation, of the constituents for forming the carrier system is carried out with the input of energy, preferably with the input of mixing and/or stirring energy, in particular into the liquid medium comprising at least a part of the constituents and/or substances of the carrier system and/or into a mixture comprising at least a part of the constituents and/or substances of the carrier system, wherein the energy input, in particular mixing and/or stirring energy, is set such that an at least substantially laminar flow is present, in particular in the liquid medium comprising at least some of the components and/or substances of the carrier system and/or in the mixture comprising at least some of the components and/or substances of the carrier system, in particular by at least substantially completely avoiding the formation of a non-laminar and/or turbulent flow and/or in particular by at least substantially completely avoiding the formation of non-laminar and/or turbulent flow components; and/or
wherein a, in particular, continuous method and/or process control (production control), in particular online and/or inline process control, is carried out, in particular for monitoring the method status and/or method progress, preferably for monitoring and/or setting the formation of the particles of the carrier system, in particular lipid nanoparticles (LNP), and/or in particular for monitoring and/or setting process parameters, such as the input of energy, preferably the input of mixing and/or stirring energy, the input and/or removal of thermal energy, flow behavior, mass throughput, volume flows or the like, wherein the method and/or process control is effected by acquiring at least one process parameter, in particular at least two process parameters, wherein the process parameter is selected from the group of temperature, conductivity, viscosity and pH value, in particular of the liquid medium, preferably together with the components and/or substances introduced therein, and/or of the composition.

According to the invention, it is in particular well proven if the following combinations are realized with regard to the aforementioned features and/or measures (1a) to (5a) with the relevant HLB values according to Griffin: Thus, according to the invention, it may in particular be provided that the method is realized by a combination of the features and/or measures (1a) and (2a), in particular by a combination of the features and/or measures (1a), (2a) and (3a), preferably by a combination of the features and/or measures (1a), (2a), (3a) and (4a) or preferably by a combination of the features and/or measures (1a), (2a), (3a) and (5a), preferably by a combination of the features and/or measures (1a), (2a), (3a), (4a) and (5a).

On the basis of the aforementioned combinations of characteristics with the defined HLB values, the method according to the invention can be further optimized, also with regard to the provision of corresponding particles of the preferably lipid-based carrier system with defined properties, in particular with regard to relatively small particle sizes with small dispersity or small variance of the underlying mean particle diameters.

In particular, the measures listed below are also beneficial in this context:
According to the invention, the substances (i) and (iii) or the substances (i) and (iv) or the substances (i) and (v) can comprise an average HLB value according to Griffin (respective arithmetic mean value of the HLB values according to Griffin of the aforementioned substance combinations) in the range from 1 to 10, in particular in the range from 2 to 9, preferably in the range from 3 to 8, or according to the invention, the substances (i) and (iii) or the substances (i) and (iv) or the substances (i) and (v) may comprise an average Log P value (respective arithmetic mean of the Log P values of the aforementioned substance combinations) in the range from 0.62 to 8.58, in particular in the range from 0.70 to 3.88, preferably in the range from 0.80 to 2.44.
Further, the substances (i), (iii) and (iv) or the substances (i), (iii) and (v) or the substances (i), (iv) and (v) may comprise an average HLB value according to Griffin (respective arithmetic mean value of the HLB values according to Griffin of the aforementioned substance combinations) in the range from 1 to 10, in particular in the range from 2 to 9, preferably in the range from 3 to 8, or furthermore, the substances (i), (iii) and (iv) or the substances (i), (iii) and (v) or the substances (i), (iv) and (v) may comprise a mean log P value (respective arithmetic mean of the log P values of the aforementioned substance combinations) of the aforementioned substance combinations) in the range from 0.62 to 8.58, in particular in the range from 0.70 to 3.88, preferably in the range from 0.80 to 2.44.
In addition, the substances (i), (iii), (iv) and (v) may comprise a mean HLB value according to Griffin (respective arithmetic mean value of the HLB values according to Griffin of the aforementioned combination of substances) in the range from 1 to 10, in particular in the range from 2 to 9, preferably in the range from 3 to 8, resp. in addition, the substances (i), (iii), (iv) and (v) may comprise a mean log P value (respective arithmetic mean of the log P values of the aforementioned substance combinations) of the aforementioned substance combinations) in the range from 0.62 to 8.58, in particular in the range from 0.70 to 3.88, preferably in the range from 0.80 to 2.44.

Consequently, it is particularly envisaged according to the invention that the amphiphilic substances, in particular based on substances (i), (iii), (iv) and (v), comprise an average HLB value according to Griffin (respective arithmetic mean value obtained according to Griffin in the aforementioned combination of substances) in the range from 1 to 10, in particular in the range from 2 to 9, preferably in the range from 3 to 8. A corresponding mean value according to Griffin also applies in particular to the further inclusion of substances listed below, which can be an optional component of the carrier system according to the invention, such as substance (vii) and/or substance (viii).

Furthermore, it may be provided according to the invention that the amphiphilic substances, in particular based on substances (i), (iii), (iv) and (v), comprise a mean Log P value (respective arithmetic mean value in the aforementioned combination of substances) in the range from 0.62 to 8.58, in particular in the range from 0.70 to 3.88, preferably in the range from 0.80 to 2.44. A corresponding Log P mean value also applies in particular to the further inclusion of substances listed below, which may be an optional component of the carrier system according to the invention, such as substance (vii) and/or substance (viii).

With regard to the substances that can be used as components of the carrier system according to the invention as part of the method according to the invention, the underlying packing parameter or critical packing parameter Pₖᵣ is also important in this respect.

The selection of the substances as components of the preferably lipid-based carrier system according to the invention using the critical packing parameter Pₖᵣ also makes it possible to control the formation or shaping of the particles on which the preferably lipid-based carrier system according to the invention is based, for example in the form of specially defined lipid nanoparticles (LNP) or in particular disk-shaped lipid nanoparticles, as will be configured below.

According to the Israelachvili model, the critical packing parameter Pₖᵣ is based on the formula Pₖᵣ=V/a₀−l_{C}, wherein l_{C} represents the length of the hydrophobic residue of the molecule on which the substance is based, V the volume of the molecule on which the substance is based and a₀ the cross-sectional surface of the hydrophilic head group of the molecule on which the substance is based. The Pₖᵣ value thus describes a ratio of hydrophilic head groups to lipophilic groups of the underlying molecule, with a focus on geometric or space-filling components. The Pₖᵣ value is dimensionless and characteristic of the molecules in question. As stated above, the formation or shaping of the particles of the preferably lipid-based carrier system according to the invention can also be effected as a function of the Pₖᵣ value.

In this context, it has proven to be advantageous in particular if the substances used according to the invention comprise the following Pₖᵣ values:
Thus, the substance (i) may comprise a critical packing parameter Pₖᵣ of at least 0.5, in particular at least 0.7, especially in the range from 0.7 to 1.225, preferably in the range from 0.77 to 1.12, preferably in the range from 0.84 to 1.05.
In addition, the substance (ii) may comprise a critical packing parameter Pₖᵣ of at most 0.35, in particular in the range from 0.007 to 0.35, preferably in the range from 0.035 to 0.315, preferably in the range from 0.07 to 0.28.
Furthermore, the substance (iii) may comprise a critical packing parameter Par of at least 1.155, in particular in the range from 1.155 to 1.225, preferably in the range from 1.162 to 1.218, preferably in the range from 1.169 to 1.211.
In addition, the substance (iv) may comprise a critical packing parameter Pₖᵣ of at least 0.98, in particular in the range from 0.98 to 1.19, preferably in the range from 1.015 to 1.155, preferably in the range from 1.05 to 1.12.
The substance (v) may comprise a critical packing parameter Pₖᵣ of at least 0.7, in particular in the range from 0.7 to 1.19, preferably in the range from 0.77 to 1.1725, preferably in the range from 0.805 to 1.155.

Against this background, the following is also particularly advantageous: Thus, the method may in particular be identified by at least one of the following features and/or measures (1b) to (5b), in particular by a combination of at least two, preferably at least three, preferably at least four, particularly preferred all five, of the following features and/or measures (1b) to (5b):
(1b) the substance (i) comprises a critical packing parameter Pₖᵣ of at least 0.5, in particular at least 0.7, preferably in the range from 0.7 to 1.225, preferably in the range from 0.77 to 1.12, preferably in the range from 0.84 to 1.05;
(2b) the substance (ii) comprises a critical packing parameter Pₖᵣ of at most 0.35, in particular in the range from 0.007 to 0.35, preferably in the range from 0.035 to 0.315, preferably in the range from 0.07 to 0.28;
(3b) wherein the substance (iii) comprises a critical packing parameter Pₖᵣ of at least 1.155, in particular in the range from 1.155 to 1.225, preferably in the range from 1.162 to 1.218, preferably in the range from 1.169 to 1.211;
(4b) the substance (iv) comprises a critical packing parameter Pₖᵣ of at least 0.98, in particular in the range from 0.98 to 1.19, preferably in the range from 1.015 to 1.155, preferably in the range from 1.05 to 1.12;
(5b) the substance (v) comprises a critical packing parameter Pₖᵣ of at least 0.7, in particular in the range from 0.7 to 1.19, preferably in the range from 0.77 to 1.1725, preferably in the range from 0.805 to 1.155.

According to the invention, the following combinations have also proven to be advantageous in particular, also with regard to the targeted control of the formation or shaping of the underlying particles: In particular, the method can be implemented by a combination of the features and/or measures (1b) and (2b), in particular by a combination of the features and/or measures (1b), (2b) and (3b), preferably by a combination of the features and/or measures (1b), (2b), (3b) and (4b) or preferably by a combination of the features and/or measures (1b), (2b), (3b) and (5b), preferably by a combination of the features and/or measures (1b), (2b), (3b), (4b) and (5b).

Furthermore, the carrier system provided as part of the method according to the invention may comprise the substances listed in particular in the amounts listed below. According to the invention, however, it may be provided in individual cases to deviate from the values listed below without departing from the present invention.

With regard to substance (i), it may behave in particular as follows:
Thus, the carrier system may comprise the substance (i) in an amount in the range from 0.5 wt. % to 50 wt. %, in particular in the range from 1 wt. % to 40 wt. %, preferably in the range from 1.25 wt. % to 30 wt. %, preferably in the range from 1.5 wt. % to 25 wt. %, based on the carrier system and/or based on the entirety of the components of the carrier system.
Similarly, the substance (i) can be used in an amount such that the carrier system comprises the substance (i) in an amount in the range from 0.1 wt. % to 50 wt. %, in particular in the range from 0.25 wt. % to 40 wt. %, preferably in the range from 0.5 wt. % to 30 wt. %, preferably in the range from 1 wt. % to 25 wt. %, based on the carrier system and/or based on the entirety of the components of the carrier system.

In addition, substance (ii) may behave as follows:
Thus, the carrier system may comprise the substance (ii) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the entirety of the components of the carrier system.
Similarly, the substance (ii) can be used in an amount such that the carrier system comprises the substance (ii) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the total of the components of the carrier system.

In addition, substance (iii) may behave as follows:
The carrier system may comprise the substance (iii) in an amount in the range from 0.2 wt. % to 40 wt. %, in particular in the range from 0.5 wt. % to 25 wt. %, preferably in the range from 1 wt. % to 20 wt. %, preferably in the range from 3 wt. % to 15 wt. %, based on the carrier system and/or based on the entirety of the components of the carrier system.
Similarly, the substance (iii) can be used in an amount such that the carrier system comprises the substance (iii) in an amount in the range from 0.2% to 40% by weight, in particular in the range from 0.5% to 25% by weight, preferably in the range from 1% to 20% by weight, preferably in the range from 3% to 15% by weight, based on the carrier system and/or based on the total of the components of the carrier system.

In addition, substance (iv) may behave as follows:
Thus, the carrier system may comprise the substance (iv) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the total of the components of the carrier system.
Similarly, the substance (iv) can be used in an amount such that the carrier system comprises the substance (iv) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the entirety of the components of the carrier system.

In addition, substance (v) can behave as follows:
Thus, the carrier system may comprise the substance (v) in an amount in the range from 2 wt. % to 85 wt. %, in particular in the range from 4 wt. % to 80 wt. %, preferably in the range from 6 wt. % to 75 wt. %, preferably in the range from 8 wt. % to 70 wt. %, based on the carrier system and/or based on the entirety of the components of the carrier system.
Similarly, the substance (v) can be used in an amount such that the carrier system comprises the substance (v) in an amount in the range from 2 wt. % to 85 wt. %, in particular in the range from 4 wt. % to 80 wt. %, preferably in the range from 6 wt. % to 75 wt. %, preferably in the range from 8 wt. % to 70 wt. %, based on the carrier system and/or based on the entirety of the components of the carrier system.

Furthermore, the weight ratio of the substances used for the carrier system or in the method according to the invention is also of great importance: On this basis, the properties of the resulting particles can be further specified or set, for example with regard to improved compatibility with the underlying liquid medium or with the active ingredient with which the particles or the carrier system are loaded or equipped.

Furthermore, in particular the following may be provided according to the invention:

Thus, the weight-related quantitative ratio of substance (i) to substance (ii) [substance (i):substance (ii)] can be in the range from 50:1 to 1:160, in particular in the range from 20:1 to 1:75, preferably in the range from 10:1 to 1:56, preferably in the range from 5:1 to 1:43, or can be set to the aforementioned values.

In particular, the substances (i) and (ii) can be used in an amount such that the carrier system comprises the substances (i) and (ii) in a weight-related amount ratio of substance (i) to substance (ii) [substance (i):substance (ii)] in the range from 50:1 to 1:160, in particular in the range from 20:1 to 1:75, preferably in the range from 10:1 to 1:56, preferably in the range from 5:1 to 1:43.

In particular, the following may also be provided according to the invention:

Thus, the weight-related quantitative ratio of substance (i) to substance (iii) [substance (i):substance (iii)] can be in the range from 250:1 to 1:80, in particular in the range from 80:1 to 1:25, preferably in the range from 30:1 to 1:16, preferably in the range from 8:1 to 1:10, or can be set to the aforementioned values.

In particular, the substances (i) and (iii) can be used in an amount such that the carrier system comprises the substances (i) and (iii) in a weight-related amount ratio of substance (i) to substance (iii) [substance (i):substance (iii)] in the range from 250:1 to 1:80, in particular in the range from 80:1 to 1:25, preferably in the range from 30:1 to 1:16, preferably in the range from 8:1 to 1:10.

Furthermore, in particular the following may be provided according to the invention:

Thus, the weight-related quantitative ratio of substance (i) to substance (iv) [substance (i):substance (iv)] can be in the range from 50:1 to 1:160, in particular in the range from 20:1 to 1:75, preferably in the range from 10:1 to 1:56, preferably in the range from 5:1 to 1:43, or can be set to the aforementioned values.

In particular, the substances (i) and (iv) can be used in an amount such that the carrier system comprises the substances (i) and (iv) in a weight-related amount ratio of substance (i) to substance (iv) [substance (i):substance (iv)] in the range from 50:1 to 1:160, in particular in the range from 20:1 to 1:75, preferably in the range from 10:1 to 1:56, preferably in the range from 5:1 to 1:43.

Further according to the invention, in particular the following can be provided:
Thus, the weight-related quantitative ratio of substance (i) to substance (v) [substance (i):substance (v)] can be in the range from 25:1 to 1:170, in particular in the range from 10:1 to 1:80, preferably in the range from 5:1 to 1:60, preferably in the range from 4:1 to 1:46, or can be set to the aforementioned values.

In particular, the substances (i) and (v) can be used in an amount such that the carrier system comprises the substances (i) and (v) in a weight-related amount ratio of substance (i) to substance (ii) [substance (i):substance (v)] in the range from 25:1 to 1:170, in particular in the range from 10:1 to 1:80, preferably in the range from 5:1 to 1:60, preferably in the range from 4:1 to 1:46.

In particular, the following may also be provided according to the invention:

Thus, the weight-based quantitative ratio of substance (i) to substance (ii) to substance (iii) to substance (iv) [substance (i):substance (ii):substance (iii):substance (iv)] may be in the range from (0.5 to 50):(1 to 80):(0.2 to 40):(1 to 80), in particular in the range from (1 to 40):(2 to 75):(0.5 to 25):(2 to 75), preferably in the range of (1.25 to 30):(3 to 70):(1 to 20):(3 to 70), preferably in the range of (1.5 to 25):(5 to 65):(3 to 15):(5 to 65), or set to the aforementioned values.

In particular, the substances (i), (ii), (iii) and (iv) can be used in an amount such that the carrier system contains the substances (i), (ii), (iii) and (iv) in a weight-related quantity ratio of substance (i) to substance (ii) to substance (iii) to substance (iv) [substance (i):substance (ii):substance (iii):substance (iv)] in the range from (0.5 to 50):(1 to 80):(0.2 to 40):(1 to 80), in particular in the range from (1 to 40):(2 to 75):(0.5 to 25):(2 to 75), preferably in the range of (1.25 to 30):(3 to 70):(1 to 20):(3 to 70), preferably in the range of (1.5 to 25):(5 to 65):(3 to 15):(5 to 65).

In this context, the following may be provided in particular according to the invention:

Thus, the weight-based quantitative ratio of substance (i) to substance (ii) to substance (iii) to substance (v) [substance (i):substance (ii):substance (iii):substance (v)] may be in the range from (0.5 to 50):(1 to 80):(0.2 to 40):(2 to 85), in particular in the range from (1 to 40):(2 to 75):(0.5 to 25):(4 to 80), preferably in the range of (1.25 to 30):(3 to 70):(1 to 20):(6 to 75), preferably in the range of (1.5 to 25):(5 to 65):(3 to 15):(8 to 70), or set to the aforementioned values.

In particular, the substances (i), (ii), (iii) and (v) can be used in an amount such that the carrier system contains the substances (i), (ii), (iii) and (v) in a weight-related quantity ratio of substance (i) to substance (ii) to substance (iii) to substance (v) [substance (i):substance (ii):substance (iii):substance (v)] in the range from (0.5 to 50):(1 to 80):(0.2 to 40):(2 to 85), in particular in the range from (1 to 40):(2 to 75):(0.5 to 25):(4 to 80), preferably in the range of (1.25 to 30):(3 to 70):(1 to 20):(6 to 75), preferably in the range of (1.5 to 25):(5 to 65):(3 to 15):(8 to 70).

Furthermore, in particular the following may be provided according to the invention:

Thus, the weight-related quantity ratio of substance (i) to substance (ii) to substance (iii) to substance (iv) to substance (v) [substance (i):Substance (ii):Substance (iii):Substance (iv):substance (v)] in the range from (0.5 to 50):(1 to 80):(0.2 to 40):(1 to 80):(2 to 85), in particular (1 to 40):(2 to 75):(0.5 to 25):(2 to 75):(4 to 80), preferably (1.25 to 30):(3 to 70):(1 to 20):(3 to 70):(6 to 75), preferably (1.5 to 25):(5 to 65):(3 to 15):(5 to 65):(8 to 70), or can be set to the aforementioned values.

In particular, the substances (i), (ii), (iii) and (iv) can be used in an amount such that the carrier system contains the substances (i), (ii), (iii) and (iv) in a weight-related quantity ratio of substance (i) to substance (ii) to substance (iii) to substance (iv) to substance (v) [substance (i):substance (ii):substance (iii):substance (iv)] in the range from (0.5 to 50):(1 to 80):(0.2 to 40):(1 to 80), in particular (1 to 40):(2 to 75):(0.5 to 25):(2 to 75), preferably (1.25 to 30):(3 to 70):(1 to 20):(3 to 70), preferably (1.5 to 25):(5 to 65):(3 to 15):(5 to 65).

According to a further preferred embodiment according to the invention, the following may also be provided:
Thus, the weight-based ratio of substance (ii) to the hydrophobic substances, in particular hydrophobic amphiphilic substances, preferably selected from the group consisting of substance (i), substance (iii), substance (iv), substance (v) and combinations thereof, preferably to substance (i) and/or substance (iii) and optionally substance (iv) and optionally substance (v) [substance (ii):hydrophobic substances] are in the range from 10:1 to 1:20, preferably in the range from 5:1 to 1:15, preferably in the range from 2:1 to 1:10, and/or are set to the aforementioned values.
In particular, the substance (ii) and the hydrophobic substances, in particular the hydrophobic amphiphilic substances, preferably selected from the group of substance (i), substance (iii), substance (iv), substance (v) and combinations thereof, can be used in an amount such that the carrier system contains the substances in a weight-related quantitative ratio of substance (ii) to the hydrophobic substances, in particular hydrophobic amphiphilic substances, preferably selected from the group of substance (i), substance (iii), substance (iv), substance (v) and combinations thereof, preferably to substance (i) and/or substance (iii) and optionally substance (iv) and optionally substance (v), substance (iv) and optionally substance (v) [substance (ii):hydrophobic substances] in the range from 10:1 to 1:20, preferably in the range from 5:1 to 1:15, preferably in the range from 2:1 to 1:10.

As far as the components for the preferably lipid-based carrier system according to the invention are concerned, the use of the substances listed below has proved to be particularly advantageous:
Thus, in particular, it may be provided according to the invention that the substance (i) is or comprises 1,2-distearoyl-sn-glycero-3-phosphocholine (DSPC, distearoylphosphatidylcholine, colfosceril stearate). In principle, lecithin, in particular soy lecithin, can be used as substance (i).

In addition, it may be provided according to the invention that the substance (ii) is and/or comprises a PEG stearate, in particular PEG 40 stearate, or a macrogol stearate, in particular macrogol 40 stearate; or that the substance (ii) is and/or comprises PEG 2000-DMG (1,2-dimyristoyl-rac-glycero-3-methoxypolyethyleneglycol-2000); or that the substance (ii) is and/or comprises a cetromacrogol (polyethylene glycol hexadecyl ether); or that the substance (ii) is and/or comprises 2-[(polyethylene glycol)-2000]-N,N-ditetradecylacetamide (ALC-0159); or that the substance (ii) is and/or comprises a polaxamer; or that the substance (ii) is or comprises an alkylpolyglycoside, in particular C₈- to C₁₄-alkylpolyglycoside, preferably C₁₀- to C₁₂-alkylpolyglycoside; or that the substance (ii) is or comprises an ethoxylated sorbitan fatty acid ester, in particular polysorbate, preferably polysorbate 80 and/or polysorbate 20.

In particular, substance (iii) may be or comprise cholesterol or phytosterol or mixtures thereof, in particular phytosterol. In addition to optimizing the composition and structure of the particles of the lipid-based carrier system, phytosterols and cholesterol are also associated with an antioxidant protective effect, which is particularly pronounced for phytosterols. This is accompanied by protection against oxidation, in particular with regard to active ingredients or other oxidation-sensitive substances or components used according to the invention. Stigmasterol, for example, can be used as a phytosterol.

Furthermore, as regards the use of calciferol (vitamin D) in particular as substance (iii), it can behave according to the invention in particular in such a way that-without wishing to limit or refer to this theory, in this respect there is an incorporation into the lamellar structure of the particles, which also leads to stabilization or oxidation protection due to the antioxidant effect of calciferol (vitamin D). Calciferol (vitamin D) can thus act in particular as a lamellar or membrane antioxidant. In addition, calciferol (vitamin D) has an immunostimulating or immunomodulating effect, so that administration can also have the effect of strengthening the immune system.

As stated above, the method according to the invention provides a universal carrier system, so to speak, which can be loaded or equipped with a large number of different types or kinds of active ingredients, wherein further tailoring can be effected on the basis of the coordination and selection of the components or substances and the controlled configuration or shaping of the particles, in particular lipid nanoparticles, of the carrier system according to the method.

With regard to the substance (iv) in the form of the cationic lipid, it is particularly the case that this is a lipid which is positively charged or formed as a cation at a pH value at least in the range from 6.5 to 7.8, in particular in the range from 6.8 to 7.6, preferably in the range from 7 to 7.5, preferably in the range from 7.35 to 7.45 (physiological range).

According to the invention, it is also provided in particular for the use of an active ingredient that the active ingredient, in particular as substance (vi), is a component of the carrier system and/or forms a component of the carrier system and/or wherein the carrier system comprises the active ingredient, in particular as substance (vi).

In particular, the active ingredient, in particular as substance (vi), can be a component of the particles, preferably lipid nanoparticles, of the preferably lipid-based carrier system in a corresponding manner. In this respect, the active ingredient, in particular as substance (vi), can be or become attached or integrated, preferably incorporated or integrated, with respect to the particles, in particular lipid nanoparticles. This leads to further protection of the active ingredient, to improved (storage) stability of the resulting composition and to improved administration of the active ingredient also at the site of action, in particular to improved uptake into (body) cells.

In particular, the following may apply to the active substance or the substance (vi) present or used as a component of the carrier system:

In particular, the active ingredient may be a therapeutic and/or prophylactic active ingredient, in particular a therapeutic and/or prophylactic pharmaceutical zeutic active ingredient.

According to the invention, the active ingredient can in particular be an at least essentially water-insoluble, preferably an at least essentially water-insoluble amphiphilic active ingredient. In this respect, the term “water-insoluble” refers in particular to the relevant property of the active ingredient at room temperature (20° C.) and ambient pressure (1,013.25 hPa). According to the invention, however, water-soluble active ingredients can also be used under the aforementioned conditions.

According to the invention, the active ingredient can be selected from the group of vaccine active ingredients (vaccination active ingredients), antibiotic active ingredients, antifungal active ingredients, antiviral active ingredients, antiphlogistic active ingredients, antipyretic active ingredients, antiallergic active ingredients, anticancer agents, hormonal agents, anticoagulant agents, antitumor agents (such as cytostatics), food supplements, vitamins, minerals, UV-absorbing substances and their mixtures and combinations, preferably vaccine agents (vaccine agents).

Furthermore, the active ingredient can be selected from the group of RNA, in particular messenger RNA (mRNA); retinol; tocopherol; corticosteroids, in particular cortisone; antibiotics, in particular tetracyclines and benzoylpenicillins; diclofenac; estradiol, estradiol hemihydrate, salicylic acid, acetylsalicylic acid; indomethacin; essential oils; in particular amphiphilic UVA filters and UVB filters; nicotinamide; and mixtures and combinations thereof.

In particular with regard to the use of RNA or mRNA, preferably single-stranded mRNA, it has been found to be advantageous according to the invention if the carrier system, in particular further comprises the substance (iv) in the form of the cationic lipid, in particular as defined above. This can provide further optimization of the equipment of the carrier system or the particles, preferably lipid nanoparticles, with the active ingredient. In particular, an optimized interaction with regard to the negatively charged or anionic RNA, in particular mRNA, can be present or ensured.

According to the invention, it may in particular be such that the active ingredient is a vaccine active ingredient (vaccine active ingredient), in particular a nucleic acid-based vaccine active ingredient and/or gene-based vaccine active ingredient, preferably a vaccine active ingredient encoding an antigen of a pathogen and/or a pathogen, in particular an antigen of a virus.

In this context, it may in particular be provided that the vaccine active ingredient is selected from the group consisting of DNA; RNA, in particular messenger RNA (mRNA); viral vectors; plasmids, in particular plasmid DNA (pDNA); preferably RNA; preferably mRNA; and mixtures and combinations thereof.

Similarly, it may be envisaged in this context that the active ingredient is an mRNA-based vaccine active ingredient and/or mRNA, in particular wherein the mRNA is single-stranded and/or in particular wherein the mRNA comprises a 5′-cap structure. In this regard, the mRNA-based vaccine active ingredient and/or the mRNA may equally comprise at least one poly(A) sequence and/or at least one poly(C) sequence.

In addition, in the context of the present invention, the active ingredient may be an mRNA-based vaccine active ingredient and/or mRNA, in particular wherein the mRNA encodes a protein, in particular antigen, of a coronavirus, preferably SARS-CoV-2, in particular wherein the protein, in particular antigen, encodes the membrane-anchored spike protein (spike glycoprotein or S-glycoprotein) of the coronavirus, preferably at least partially, preferably at least substantially completely. S protein or S glycoprotein) of the coronavirus, preferably SARS-CoV-2, at least partially, preferably at least essentially completely. In this context, it may in particular be a single-stranded, 5′-capped messenger RNA (mRNA) that codes for the membrane-anchored viral spike (S) protein or spike (S) glycoprotein of SARS-CoV-2. In general, the active ingredient may be a gene-based active ingredient or gene-based vaccine active ingredient. According to the invention, however, the active ingredient or vaccine active ingredient can also be a protein and/or antigen, in particular of a pathogenic pathogen, such as a bacterium, virus or the like.

In general, the active ingredient or substance (vi) may comprise an HLB value according to Griffin in the range from 0.5 to 6, in particular in the range from 1 to 5. In contrast, however, the active ingredient or substance (vi) may also comprise an HLB value according to Griffin in the range from 6 to 15, in particular in the range from 8 to 14, preferably in the range from 10 to 13. For mRNA, in particular, an HLB value according to Griffin in the range from 8 to 14, especially 10 to 13, may be present. In addition, the active ingredient or substance (vi) may comprise a Log P value in the range from 1.11 to 18.97, in particular in the range from 1.36 to 8.58. In contrast, however, the active ingredient or substance (vi) may also comprise a Log P value in the range from 0.39 to 1.11, in particular in the range from 0.42 to 0.80, preferably in the range from 0.46 to 0.62. For mRNA, a Log P value in the range of 0.42 to 0.80, in particular 0.46 to 0.62, may be present.

According to the invention, the carrier system may comprise the active ingredient, in particular as substance (vi), or the substance (vi) in an amount in the range from 0.01 wt. % to 80 wt. %, in particular in the range from 0.1 wt. % to 70 wt. %, preferably in the range from 0.5 wt. % to 60 wt. %, preferably in the range from 1 wt. % to 50 wt. %, based on the carrier system and/or based on the totality of the components of the carrier system.

In this context, the ratio of the active ingredient or substance (vi) to the other components or substances of the carrier system is also important:

Thus, the weight-based quantitative ratio of substance (i) to substance (ii) to active ingredient or substance (vi) (i.e. the active ingredient) [substance (i):substance (ii):substance (vi)] can be in the range of (0.5 to 50):(1 to 80):(0.01 to 80), in particular in the range of (1 to 40):(2 to 75):(0.1 to 70), preferably in the range of (1.25 to 30):(3 to 70):(0.5 to 60), preferably in the range of (1.5 to 25):(5 to 65):(1 to 50) and/or set to the aforementioned values.

Furthermore, the weight-related quantity ratio of substance (i) to substance (ii) to substance (iii) to substance (iv) to substance (v) to active ingredient or substance (vi) [substance (i):Substance (ii):Substance (iii):Substance (iv):Substance (v):Substance (vi)] in the range from (0.5 to 50):(1 to 80):(0.2 to 40):(1 to 80):(2 to 85):(0.01 to 80), in particular in the range from (1 to 40):(2 to 75):(0.5 to 25):(2 to 75):(4 to 80):(0.1 to 70), preferably in the range of (1.25 to 30):(3 to 70):(1 to 20):(3 to 70):(6 to 75):(0.5 to 60), preferably in the range of (1.5 to 25):(5 to 65):(3 to 15):(5 to 65):(8 to 70):(1:50), and/or set to the aforementioned values.

In addition, the lipid-based carrier system in particular may comprise other components or substances which can be used in the method according to the invention. In this way, the properties of the resulting particles or lipid nanoparticles can be further set and the formation of the particles can be further optimized as part of the process operation:

Erfindungsgemäß kann das Trägersystem als weiteren Bestandteil, insbesondere als Substanz (vii), mindestens einen Fettalkohol, insbesondere C₈-bis C₂₅-Fettalkohol, vorzugsweise mindestens einen verzweigten Fettalkohol, bevorzugt Octyldodecanol, besonders bevorzugt-2Octyldocen-1-ol, umfassen, insbesondere in einer Menge im Bereich von 1 Gew.-% to 80% by weight, preferably in the range from 5% to 70% by weight, preferably in the range from 10% to 60% by weight, based on the carrier system and/or based on the total of the components of the carrier system.

In this context, the fatty alcohol, preferably octyldodecanol, may comprise an HLB value according to Griffin in the range from 1 to 5, in particular in the range from 2 to 4, and/or the fatty alcohol, preferably octyldodecanol, may comprise a Log P value in the range from 1.36 to 8.58, in particular in the range from 1.76 to 3.88.

Similarly, the fatty alcohol, preferably octyldodecanol, may comprise a critical packing parameter Pₖᵣ in the range from 0.91 to 1.19, in particular in the range from 0.98 to 1.12.

In particular, the fatty alcohol or octyldodecanol can be 2-octyldodecan-1-ol (trade name Eutanol G). Octyldecanol comprises an HLB value according to Griffin of about 3 and is therefore very similar in its physical properties to the previously mentioned substance ALC-0315. The use of a fatty alcohol can be used to further control the formation of the carrier system in the form of particles.

Furthermore, the carrier system can comprise as a further component, in particular as substance (viii), at least one surface-active amphiphilic substance, in particular surface-active and non-micelle-forming amphiphilic substance, preferably at least one sorbitan fatty acid ester, preferably at least one sorbitan fatty acid ester with or based on saturated and/or unsaturated C₈- to C₁₈-fatty acids, in particular C₁₀- to C₁₄-fatty acids, particularly preferred sorbitan caprylate, in particular in an amount in the range of 0.1 wt.-% to 30 wt.-%, preferably at least one sorbitan fatty acid ester, preferably at least one sorbitan fatty acid ester with or based on saturated and/or unsaturated C- to C-fatty acids, in particular C- to C-fatty acids, particularly preferred sorbitan caprylate, in particular in an amount in the range of 0.1 wt.-% to 30 wt. %, preferably in the range from 1 wt. % to 20 wt. %, preferably in the range from 5 wt.-% to 15 wt. %, based on the carrier system and/or based on the total of the components of the carrier system.

In this context, the surface-active amphiphilic substance, preferably the sorbitan fatty acid ester, may comprise an HLB value according to Griffin in the range from 2 to 5, in particular in the range from 3 to 4, or the surface-active amphiphilic substance, preferably the sorbitan fatty acid ester, may comprise a Log P value in the range from 1.36 to 3.88, in particular in the range from 1.76 to 2.44.

In addition, the surface-active amphiphilic substance, preferably the sorbitan fatty acid ester, may comprise a critical packing parameter Pₖᵣ in the range from 0.91 to 1.12, in particular in the range from 0.98 to 1.05.

In addition, the carrier system may comprise as a further component, in particular as substance (ix), at least one ether, in particular ether of a diol or polyol, preferably ether of a bicyclic diol, preferably dialkyl isosorbide, particularly preferably dimethyl isosorbide (DMI), even more preferably 2,5-dimethyl isosorbide, in particular in an amount in the range from 2.5 wt.-% to 30 wt.-%, preferably in the range from 5 wt.-% to 25 wt.-%, preferably in the range from 10 wt.-% to 20 wt.-%, based on the carrier system and/or % to 30 wt. %, preferably in the range from 5 wt. % to 25 wt. %, preferably in the range from 10 wt. % to 20 wt. %, based on the carrier system and/or based on the total of the components of the carrier system.

Without wishing to limit or refer to this theory, dimethyl isosorbide is a penetration enhancer, which can facilitate the uptake of the active substance into a (body) cell. In addition, the targeted use of dimethyl isosorbide in producing the particle system means that particularly small particles, preferably lipid nanoparticles, are obtained in relation to the carrier system, and this with low dispersity or low variance at the same time.

According to the invention, it may further be provided that the carrier system comprises as a further component, in particular as substance (x), at least one alcohol, in particular wherein the alcohol is selected from the group of monohydric alcohols, polyhydric alcohols, in particular polyols, and mixtures thereof and combinations thereof; and/or wherein the carrier system comprises as a further component, in particular as substance (x), at least one polyol.

In this context, it may be provided that the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures thereof.

Similarly, it may be provided in this respect that the alcohol, in particular the polyol, is selected from the group of propylene glycol, polyethylene glycol, sorbitol, mannitol, glycerol, sucrose, panthenol and mixtures and combinations thereof.

According to the invention, it is preferably that the alcohol, in particular the polyol, is panthenol. Panthenol is also a penetration enhancer. In this respect, the applicant has found in a completely surprising way that the penetration behavior of the resulting particles, in particular lipid nanoparticles of the carrier system, can be predetermined or set by the targeted and purposeful use of panthenol, as is already the case for the previously described dimethylisosorbide according to substance (ix).

Overall, panthenol can comprise multifunctional properties with regard to the use according to the invention. Panthenol is a cationic substance that is water-soluble but also anchors itself in lipid-based structures or interacts with them.

According to the invention, the alcohol, in particular the polyol, can also be glycerol.

According to the invention, the substance (x) or the alcohol, in particular the polyol, can be present or used in an amount in the range from 1 wt. % to 90 wt. %, in particular in the range from 2 wt. % to 70 wt. %, preferably in the range from 3 wt. % to 60 wt. %, preferably in the range from 5 wt. % to 55 wt. %, based on the carrier system, in particular based on the entirety of the components of the carrier system.

According to the invention, it may also be provided that the alcohol, in particular the polyol, is used in an amount such that the weight-related quantitative ratio of alcohol, in particular the polyol, to carrier system, in particular the entirety of the components of the carrier system, [alcohol, in particular polyol:carrier system, in particular the entirety of the components of the carrier system] is in the range from 1:150 to 10:1, in particular in the range from 1:100 to 5:1, preferably in the range from 1:50 to 1:1, preferably in the range from 1:20 to 1:5, and/or is set.

According to a preferred embodiment according to the invention, it is in particular provided that the alcohol is not a C₁- to C₄-monoalcohol, preferably not an ethanol, and/or in particular wherein the alcohol does not comprise a C₁- to C₄-monoalcohol, in particular not an ethanol. In particular, the substance (x) and/or the carrier material is not a C₁- to C₄-monoalcohol, preferably not an ethanol. Preferably, the substance (x) does not comprise any C₁- to C₄-monoalcohol, in particular no ethanol.

According to the invention, it is provided in a particularly preferred manner that the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant).

In particular, in this context, it is envisaged that the liquid medium is formed and/or present as a continuous phase.

In particular, it is equally envisaged that the formation of the carrier system, in particular in particulate form, preferably in the form of lipid nanoparticles (LNP), is effected and/or brought about in the liquid medium.

According to the invention, it behaves in particular in such a way that after formation of the carrier system a preferably disperse composition, in particular a dispersion, which comprises the carrier system or the carrier system loaded and/or equipped with the active pharmaceutical ingredient and the liquid medium, results and/or in particular wherein after formation of the carrier system a dispersion of the carrier system or the carrier system loaded and/or equipped with an active pharmaceutical ingredient results in the liquid medium.

Accordingly, according to the invention, it is thus in particular provided that the contacting or interaction of the components to form the carrier system is effected in a liquid medium, in particular for the purpose of agglomeration or aggregation of the components to form the particles of the carrier system, in particular lipid nanoparticles.

In other words, according to the invention, it is particularly the case that the components of the carrier system are brought into contact (with each other) and/or into interaction (with each other) in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), or that the agglomeration of the components to form the carrier system, preferably in particulate form, preferably in the form of lipid nanoparticles (LNP), is effected and/or brought about in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant).

In this context, the liquid medium can be formed or present as a continuous phase. In addition, it also behaves in particular in this respect in such a way that after formation of the carrier system a preferably disperse composition, in particular a dispersion, which comprises the carrier system or the carrier system loaded and/or equipped with the active pharmaceutical ingredient and the liquid medium, results and/or in particular wherein after formation of the carrier system a dispersion of the carrier system or the carrier system loaded and/or equipped with the active pharmaceutical ingredient results in the liquid medium.

With regard to the process operation according to the invention and the associated formation of the carrier system in the form of particles, in particular lipid nanoparticles, the further formation of the liquid medium and the amount used in the process according to the invention, in particular also in relation to the components of the carrier system, are also of important significance:

In particular, the liquid medium can be present or used in an amount 0.05 times to 10 times, in particular 0.1 times to 5 times, in particular 0.15 times to 4 times, preferably 0.2 times to 3 times, the amount by weight of the carrier system, in particular the amount by weight of the entirety of the components of the carrier system. In particular, the amount by weight of the liquid medium refers to the entirety of the liquid medium with any further components or constituents of the liquid medium provided therein. In accordance with the invention, it can also be provided in a corresponding manner that the liquid medium is used in an amount such that the weight-related quantity ratio of carrier system, in particular the entirety of the components of the carrier system, to liquid medium [carrier system, in particular the entirety of the components of the carrier system:liquid medium] is in the range from 1:20 to 50:1, in particular in the range from 1:10 to 20:1, preferably in the range from 1:5 to 10:1, preferably in the range from 1:4 to 5:1, and/or is set.

According to the invention, the liquid medium may further comprise water and/or at least one alcohol, preferably water and at least one alcohol. In this context, the alcohol may be selected from the group consisting of monohydric alcohols, polyhydric alcohols, in particular polyols, and mixtures and combinations thereof.

In a preferred manner according to the invention, the alcohol is a polyhydric alcohol, in particular a polyol. By using the alcohols provided in this respect, in particular polyhydric alcohols, such as polyols, the penetration or dispersion of the components in the liquid medium can be controlled or improved, which is also advantageous with regard to the formation of the particulate structures in the liquid medium. As described below, it is particularly the case according to the invention that the alcohol in question is not a C-C₁₄-monoalcohol, in particular not ethanol.

In contrast, it is preferably according to the invention that the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof.

According to the invention, it is particularly provided that the alcohol, in particular the polyol, is selected from the group of propylene glycol, polyethylene glycol, sorbitol, mannitol, glycerol, sucrose, panthenol and mixtures thereof and combinations thereof. According to a preferred embodiment, the alcohol, in particular the polyol, can be panthenol. Thus, in principle, the liquid medium can also comprise panthenol, and in particular independently of the components of the carrier system. In this respect, reference can also be made to the above configurations with regard to the properties of panthenol.

In addition, the alcohol, in particular the polyol, can also be glycerol. In the context of the present invention, glycerol comprises surprisingly special properties with regard to its use for the liquid medium described herein or for the carrier system (cf. substance (x) described above). In particular, higher-melting substances, especially higher-melting amphiphilic substances, such as higher-melting phospholipids (e.g. DSPC), can also be dissolved or optimally brought into interaction with each other or with the other substances using glycerol.

The alcohol in the liquid medium also behaves in particular according to a particularly preferred embodiment in such a way that the alcohol is not a C₁- to C₄-monoalcohol, preferably not ethanol, or that the alcohol does not comprise a C₁- to C₄-monoalcohol, in particular not ethanol. In other words, the liquid medium also behaves in particular in such a way that it does not comprise or comprise any C₁- to C₄-monoalcohol, in particular no ethanol, and is thus in particular free of a C₁- to C₄-monoalcohol.

According to the invention, the liquid medium may comprise the water in an amount in the range from 10 wt. % to 99.5 wt. %, in particular in the range from 20 wt. % to 99 wt. %, preferably in the range from 30 wt. % to 97 wt. %, preferably in the range from 35 wt. % to 95 wt. %, based on the liquid medium. In particular, the liquid medium may comprise the alcohol, in particular the polyol, in an amount in the range from 0.5% to 90% by weight, in particular in the range from 1% to 80% by weight, preferably in the range from 3% to 70% by weight, preferably in the range from 5% to 65% by weight, based on the liquid medium.

In addition, the liquid medium may comprise or consist of a mixture of water and at least one alcohol, in particular polyol, in particular as defined above, wherein the weight-related quantitative ratio of water to alcohol, in particular polyol, [water:alcohol, in particular polyol] is in the range from 200:1 to 1:50, in particular in the range from 100:1 to 1:20, preferably in the range from 50:1 to 1:5, preferably in the range from 20:1 to 1:1, in particular with respect to the liquid medium.

Against this background, it is particularly envisaged according to the invention that the method, in particular the producing of the carrier system or the carrier system loaded and/or equipped with a pharmaceutically active substance, is carried out in the presence of at least one alcohol, in particular polyol, in particular as a component of the liquid medium and/or as a component of the carrier system, or that the agglomeration of the components to form the carrier system, preferably in particulate form, preferably in the form of lipid nanoparticles (LNP), is carried out in the presence of at least one alcohol, in particular polyol.

In this regard, it is in particular provided that the alcohol is selected from the group of monohydric alcohols, polyhydric alcohols, in particular polyols, and mixtures and combinations thereof, in particular as defined above, or that the alcohol, in particular the polyol, is present or used in an amount as defined above.

According to the invention, it may further be provided that the liquid medium comprises at least one ether, in particular ether of a diol or polyol, preferably ether of a bicyclic diol, preferably dialkyl isosorbide, particularly preferably dimethyl isosorbide, even more preferably 2,5-dimethyl isosorbide, in particular in an amount in the range from 0.5 wt.-% to 25 wt.-%, preferably in the range from 1 wt.-% to 10 wt.-%, based on the liquid medium. % to 25 wt. %, preferably in the range from 1 wt. % to 10 wt. %, preferably in the range from 1 wt. % to 10 wt. %, based on the liquid medium.

In particular, the method according to the invention, especially the producing of the carrier system or the carrier system loaded and/or equipped with a pharmaceutically active agent, can be carried out in the presence of at least one ether, in particular as defined above, or the agglomeration of the components to form the carrier system, preferably in particulate form, preferably in the form of lipid nanoparticles (LNP), can be carried out in the presence of at least one ether, in particular as defined above. In this regard, the ether may be present or used in an amount as previously defined.

According to a further preferred embodiment, it may also be provided according to the invention that the liquid medium comprises at least one antioxidant, in particular ascorbic acid (vitamin C), in particular in an amount in the range from 0.1 wt. % to 10 wt. %, preferably in the range from 0.2 wt. % to 5 wt. %, preferably in the range from 0.5 wt. % to 2 wt. %, based on the liquid medium.

According to the invention, it may in particular be provided that the method, in particular the producing of the carrier system or the carrier system loaded and/or equipped with a pharmaceutical active ingredient, is carried out in the presence of at least one antioxidant, in particular ascorbic acid (vitamin C), in particular as defined above; or the agglomeration of the components to form the carrier system, preferably in particulate form, preferably in the form of lipid nanoparticles (LNP), is carried out in the presence of at least one antioxidant, in particular ascorbic acid (vitamin C). In this respect, the antioxidant may be present or used in an amount as defined above.

Furthermore, the liquid medium may comprise at least one biopolymer, in particular selected from the group of alginates, hyaluronic acid and chitosan (as well as their respective salts and derivatives) and mixtures and combinations thereof, in particular in an amount in the range from 0.1 wt. % to 50 wt. %, in particular in the range from 0.5 wt. % to 30 wt. %, preferably in the range from 1 wt. % to 10 wt. %, based on the liquid medium. The use of the aforementioned biopolymers is considered in particular in the case where the liquid medium is further used for (subsequent) dilution of the carrier system or the particles. This can provide the composition with additional properties.

Furthermore, the liquid medium may comprise further components or ingredients, such as additives or the like, in particular selected from the group of wetting agents or surface-active substances, preservatives, stabilizers, taste- or odour-modifying substances, rheology modifiers, thickeners, dyes, buffers, pH adjusters or mixtures or combinations thereof. In this regard, flavorings such as (further) sweeteners or sweeteners can also be used. For example, acesulfame can be added as a sweetener (e.g. to counteract a bitter taste when the composition is administered orally).

As also stated above, it may be in accordance with the invention that the carrier system and/or the liquid medium and/or the composition, in particular as defined above, is/are at least substantially free of aliphatic monoalcohols, in particular at least substantially free of aliphatic C₁- to C₄-monoalcohols, preferably at least substantially free of ethanol. In this respect, the method according to the invention may in particular be carried out in the at least substantially complete absence of aliphatic monoalcohols, in particular in the at least substantially complete absence of aliphatic C₁- to C₄-monoalcohols, preferably in the at least substantially complete absence of ethanol. In this way, a high compatibility with respect to the active ingredients and a high compatibility are ensured. In particular, there is no need for complex method steps to remove the monoalcohol mentioned above.

Moreover, according to the invention, it is in particular provided that the carrier system and/or the liquid medium and/or the composition, in particular as defined above, is or are at least substantially free of halogenated hydrocarbons, in particular at least substantially free of chlorinated hydrocarbons, preferably at least substantially free of chloroform. In particular, the method can be carried out in at least substantially complete absence of halogenated hydrocarbons, in particular in at least substantially complete absence of chlorinated hydrocarbons, preferably in at least substantially complete absence of chloroform.

With regard to the particles of the carrier system produced or provided by the special process operation, the following can also be cited in particular.

In particular, the agglomeration, especially aggregation, of the components is effected in such a way that the carrier system results in particulate form, in particular in the form of lipid nanoparticles (LNP), preferably lipid nanoparticles (LNP) in the form of micelles, vesicles and/or disk-shaped lipid nanoparticles (LNP) (disk lipid nanoparticles or disk LNP), disk-LNP), preferably disk-shaped lipid nanoparticles (LNP). According to the invention, the carrier system is in particular in particulate form, in particular in the form of lipid nanoparticles (LNP), preferably lipid nanoparticles (LNP) in the form of micelles, vesicles and/or discoidal (disk-shaped) lipid nanoparticles (LNP) (disk lipid nanoparticles or disk LNP), disk-LNP), preferably disk-shaped lipid nanoparticles (LNP). According to the invention, it is in particular the case that discoid (disk-shaped) lipid nanoparticles are formed in particular and preferably due to the special process operation. In principle, however, other spatial structures or particle shapes may also be present, for example in the form of micelles, in particular spherical micelles and/or worm micelles, vesicles or the like.

The aforementioned formation or shaping of the particles is of special advantage both with regard to the rheological properties, the (storage) stability of the composition and the equipping of the particles with the active ingredient and its release at the site of action, and in particular with regard to the formation of the particles in the form of disk-shaped lipid nanoparticles (synonymously also referred to as disk lipid nanoparticles). The formation of the aforementioned particles is also controlled in particular by the selection and coordination of the components or substances used for the carrier system and their processing in accordance with the process. Due to their special shape, the disk-shaped lipid nanoparticles in question comprise particularly good properties with regard to the absorption or integration of active ingredients and their targeted release and also lead to particularly homogeneous and stable compositions or dispersions.

In principle, the particles can in particular be present or formed as a preferably homogeneous and/or preferably coherent lamellar structure or shape. On the other hand, a formation or presence in hexagonal or cubic form is also possible.

In particular, the method according to the invention can also be used in this context in such a way that the formation of, in particular, large multilayer vesicles (MLVs) is at least substantially completely avoided or at least reduced in the course of producing the carrier system or the particles in question.

The method according to the invention or the carrier system obtainable in this way is also characterized in particular by the fact that the particles of the carrier system, preferably lipid nanoparticles (LNP), comprise an at least substantially homogeneous particle size distribution, in particular with low dispersity and/or with small variance. In particular, the method according to the invention ensures that the particles of the carrier system, preferably lipid nanoparticles (LNP), comprise at least substantially a normal distribution and/or Gaussian distribution, in particular with low dispersity and/or with small variance.

In this context, the relevant particle sizes, in particular the hydrodynamic diameters, can be determined using light diffraction (light diffractometry), in particular laser diffraction, preferably dynamic light scattering (DLS), preferably in accordance with ISO 22412:2017 and/or DIN ISO 22412:2018-09.

In particular, it can behave according to the invention in such a way that the particle sizes (absolute), in particular the hydrodynamic diameters (absolute) of the particles of the carrier system, preferably lipid nanoparticles, comprise at least essentially a normal distribution and/or standard distribution. In this way, special defined properties of the composition obtained in this respect are obtained or guaranteed.

In general, the method according to the invention leads to the formation of particles with small particle sizes or particle diameters, which are in particular in the nanometer range.

In particular, the particles of the carrier system, preferably the lipid nanoparticles (LNP), can have a particle size (absolute), in particular a hydrodynamic diameter (absolute), in the range from 5 nm to 10,000 nm, in particular in the range from 10 nm to 5,000 nm, preferably in the range from 20 nm to 3,000 nm, preferably in the range from 50 nm to 2,000 nm, particularly preferred in the range from 75 nm to 1.500 nm, particularly more preferably in the range from 90 nm to 1,250 nm, further preferably in the range from 100 nm to 1,000 nm, again further preferably in the range from 100 nm to 900 nm, determined in particular using light diffraction (light diffractometry), in particular laser diffraction, preferably dynamic light scattering (DLS), preferably in accordance with ISO 22412:2017 and/or DIN ISO 22412:2018-09.

For example, particle sizes can be determined using a Horiba® LA-920 Particle Size Analyzer.

In particular, the particles of the carrier system, preferably the lipid nanoparticles (LNP), can have an average particle size, in particular an average hydrodynamic diameter, in the range from 5 nm to 2,900 nm, in particular in the range from 10 nm to 2,500 nm, preferably in the range from 20 nm to 2.000 nm, preferably in the range from 50 nm to 1,500 nm, particularly preferred in the range from 50 nm to 1,000 nm, in particular determined using light diffraction (light diffractometry), in particular laser diffraction, preferably dynamic light scattering (DLS), preferably in accordance with ISO 22412:2017 and/or DIN ISO 22412:2018-09.

As previously stated, the particles may comprise an overall low variance: In this respect, according to the invention, it may in particular be the case that the variance of the particle size, in particular the variance of the hydrodynamic diameter, of the particles of the carrier system, preferably the lipid nanoparticles (LNP), is at most 45%, in particular at most 35%, preferably at most 25%, preferably at most 20%, especially preferably at most 15%, even more preferably at most 10%, in particular with respect to the mean particle size or the mean hydrodynamic diameter and/or in particular determined using light diffraction (light diffractometry), in particular laser diffraction, preferably dynamic light scattering (DLS), preferably in accordance with ISO 22412:2017 and/or DIN ISO 22412:2018-09. In other words, the particles of the carrier system, preferably the lipid nanoparticles, may comprise a variance of the particle size, in particular a variance of the hydrodynamic diameter, of at most 45%, in particular at most 35%, preferably at most 25%, preferably at most 20%, particularly preferably at most 15%, particularly more preferably at most 10%, in particular based on the mean particle size or the mean hydrodynamic diameter and/or in particular determined using light diffraction (light diffractometry), in particular as stated above.

In particular, the particles of the carrier system, preferably the lipid nanoparticles (LNP), can have an average particle size D50, in particular an average hydrodynamic diameter D50, in the range from 2.5 nm to 950 nm, in particular in the range from 7.5 nm to 850 nm, preferably in the range from 10 nm to 800 nm, preferably in the range from 15 nm to 750 nm, in particular determined using light diffraction (light diffractometry), in particular laser diffraction, preferably dynamic light scattering (DLS), preferably in accordance with ISO 22412:2017 and/or DIN ISO 22412:2018-09.

In addition, the particles of the carrier system, preferably the lipid nanoparticles (LNP), can have an average particle size D90, in particular an average hydrodynamic diameter D90, in the range from 1 nm to 1.000 nm, in particular in the range from 5 nm to 900 nm, preferably in the range from 7.5 nm to 850 nm, preferably in the range from 10 nm to 800 nm, in particular determined using light diffraction (light diffractometry), in particular laser diffraction, preferably dynamic light scattering (DLS), preferably in accordance with ISO 22412:2017 and/or DIN ISO 22412:2018-09.

In the following, the method according to the invention is described in further detail with reference to preferred embodiments, on the basis of which it is further possible to obtain or set the previously defined particle shapes and sizes.

As part of the process operation according to the invention, the agglomeration, in particular aggregation, of the components (in particular to form the carrier system, preferably in particulate form) can be effected in such a way that a lyotropic liquid-crystalline structure based in particular on the carrier system and the liquid medium results. In particular, the carrier system together with the liquid medium can be present as a lyotropic liquid-crystalline structure based on the carrier system and the liquid medium and/or comprise this structure. In particular, a corresponding, in particular lyotropic, liquid-crystalline structure or phase can be obtained in the liquid medium as part of the process operation according to the invention with the inclusion of the components of the carrier system. In this context, a gel phase can be present or result in particular with regard to the liquid medium with the components present therein.

As part of the process operation according to the invention, the agglomeration, in particular aggregation, of the constituents to form the carrier system can in particular be carried out with input of energy, preferably input of mixing and/or stirring energy, in particular into the liquid medium comprising at least some of the constituents and/or substances of the carrier system and/or into a mixture comprising at least some of the constituents and/or substances of the carrier system.

In this respect, the energy introduced, in particular mixing and/or stirring energy, can be set such that an at least substantially laminar flow is present, in particular in the liquid medium comprising at least some of the components and/or substances of the carrier system and/or in the mixture comprising at least some of the components and/or substances of the carrier system, in particular with at least substantially complete avoidance of the formation of a non-laminar and/or turbulent flow and/or in particular with at least substantially complete avoidance of the formation of non-laminar and/or turbulent flow components.

In the context of the invention, it is possible, for example, to proceed in such a way that the components or substances used to form the carrier system are supplied in their entirety (for example simultaneously or successively) into the liquid medium and a corresponding input of energy is effected. However, as will be explained in detail below, it is also possible to proceed according to the invention in such a way that the components or substances used to form the carrier system are divided up, in particular in different batches, wherein mixtures of components without a liquid medium may also be present, in which the introduction of energy can take place. In this respect, batches of at least some of the components or substances can also be produced with a liquid medium, in which an input of energy can also be effected. The batches in question can then be combined, wherein in this respect another input of energy and, if necessary, the addition of further liquid medium can be effected to obtain the composition.

According to the invention, priority is thus given to setting laminar flow conditions. In this context, the applicant has found in a completely surprising manner that the presence of laminar flows in the fluid medium with the components of the carrier system introduced with it leads to the formation of special defined particulate structures, in particular as defined above, for example with regard to obtaining the discoidal lipid nanoparticles in question. As a result of the presence of a laminar flow or by mixing the components, the active ingredient to be supplied is in particular also protected, for example with regard to reducing the influence of shear forces or the like, which is particularly important for active ingredients based on mRNA or the like.

In general, the amount of energy introduced within the scope of the method according to the present invention should be effected in particular within the following ranges or under the following conditions. In individual cases, however, it is also possible to deviate from the following values within the scope of the present invention without departing from the present invention:
In particular, the amount of energy introduced, especially the amount of mixing and/or stirring energy introduced, can be in the range from 0.1 W/kg to 10 W/kg, in particular in the range from 0.2 W/kg to 5 W/kg, preferably in the range from 0.5 W/kg to 3 W/kg, preferably in the range from 1 W/kg to 2 W/kg, relative to the components to be brought into interaction, optionally together with the liquid medium.
Further, the amount of energy introduced, in particular the amount of mixing and/or stirring energy introduced, can be at most 2,000 J/kg, in particular at most 1,000 J/kg, preferably at most 500 J/kg, preferably at most 200 J/kg, relative to the components to be brought into interaction, optionally together with the liquid medium.
In particular, the amount of energy introduced, in particular the amount of mixing and/or stirring energy introduced, can be set such that a Reynolds number Rₑ, in particular a Reynolds number Rₑ related to a stirrer flow, of at most 2,000, in particular at most 1,500, preferably at most 1,000, preferably at most 500, is present.
In particular, the input of energy, in particular the input of mixing and/or stirring energy, can be effected for a period of time in the range from 0.1 s to 600 s, in particular in the range from 1 s to 500 s, preferably in the range from 2 s to 400 s, preferably in the range from 5 s to 200 s.
According to the invention, it can also be provided that the input of energy, in particular the input of mixing and/or stirring energy, is effected using at least one stirring device (stirring device), in particular wherein the stirring device is a wall-mounted stirring device and/or in particular wherein the stirring device is a spiral stirrer or a wire stirrer and/or in particular wherein the stirring device is part of a receiving device (processing device), in particular a container and/or reactor, preferably a stirring chamber and/or stirring vessel, preferably a wire stirrer, and/or in particular wherein the stirring device is a component of a receiving device (processing device), in particular a container and/or reactor, preferably a stirring chamber and/or stirring vessel.
In particular, the particle size, in particular the mean particle size, and/or the variance of the particle size of the particles, in particular lipid nanoparticles (LNP), of the carrier system can be controlled or set via the time duration of the input of energy, in particular via the time duration of the input of mixing and/or stirring energy, and/or via the amount of energy introduced, in particular the amount of mixing and/or stirring energy introduced, wherein larger time durations and/or larger amounts of energy lead to smaller particle sizes and/or smaller variances.

According to the invention, it is advantageous if the containers or reactors used according to the invention are in particular tubular or boiler-shaped. In particular, this allows dead spaces to be avoided. For example, so-called Ekato-Paravisc®-stirring devices can be used. The laminar flow can be controlled or monitored, for example, via the speed of the stirring device or the viscosity values of the fluid.

With regard to the supply of, in particular, mixing or stirring energy, the applicant has found in a completely surprising way that both the median of the particle size and the variance depend on the amount of energy supplied and on the stirring duration with regard to the coherent liquid crystalline structures formed or to be formed, or can be controlled thereby. In particular, the median particle size decreases exponentially with increasing stirring time. At the same time, the variance also decreases, in particular on a logarithmic scale. In particular, the agitation time also determines the particle size or the underlying variance. The longer stirring is carried out taking laminar flow into account, the smaller the particles become, in particular with a simultaneous decrease in variance. This allows the particle sizes or the relevant compositions to be set in a targeted manner.

With regard to the amount of energy introduced, the so-called Reynolds number is also of great importance, as mentioned above. The Reynolds number is a characteristic value for the amount of energy introduced into the underlying mixture or system.

The Reynolds number can be described in particular according to the following formula:

$$Re = \frac{ρ \cdot N \cdot {D}^{2}}{η}$$

η=dynamic viscosity [Pa·s]; ρ=density of the fluid [kg/m³], N=speed of the agitator [1/s]; D=diameter of the agitator [m])

According to the invention, it may in particular be provided that the energy input is not effected via ultrasound. This avoids an uneven and difficult to control input of energy, so that, according to the invention, a special gentle producing process is ensured.

According to the invention, the method, in particular the producing of the carrier system or the carrier system loaded and/or equipped with the active pharmaceutical ingredient, and/or the agglomeration of the components to form the carrier system can be carried out or effected at a temperature of at most 100° C., in particular at most 95° C., preferably at most 90° C.

In particular, the method, especially the producing of the carrier system or the carrier system loaded and/or equipped with the active pharmaceutical ingredient, and/or the agglomeration of the components for forming the carrier system can be carried out or effected at a temperature in the range from 10° C. to 100° C., in particular in the range from 15° C. to 95° C., preferably in the range from 20° C. to 90° C.

According to the invention, it is advantageous if the method, in particular the producing of the carrier system or the carrier system loaded and/or equipped with the active pharmaceutical ingredient, and/or the agglomeration of the components for forming the carrier system is carried out and/or effected at a temperature below the phase transition temperature of the (i) phospholipid. In particular, the process temperature, in particular the temperature for producing the carrier system or the carrier system loaded and/or equipped with the active pharmaceutical ingredient, and/or the temperature for agglomeration of the components for forming the carrier system can be selected and/or set as a function of the (i) phospholipid used, preferably to a temperature value below the phase transition temperature of the (i) phospholipid. In this way, special stable and homogeneously formed carrier systems can be obtained.

According to the invention, the method, in particular the producing of the carrier system or the carrier system loaded and/or equipped with the active pharmaceutical ingredient, and/or the agglomeration of the components to form the carrier system can be carried out continuously, semi-continuously or batchwise, in particular continuously, preferably continuously-cascaded.

In particular, the method, especially the producing of the carrier system or the carrier system loaded and/or equipped with the active pharmaceutical ingredient, and/or the agglomeration of the components for forming the carrier system can be carried out in at least one receiving device, in particular a container and/or reactor, preferably a stirring chamber and/or stirring vessel. No corresponding processing takes place in the receiving device.

In this respect, as stated in the present case, the method can, for example, be based on a joint batch of the components or the liquid medium or on different batches, for example by dividing the components and possibly in combination with liquid medium in a respective batch, in particular followed by a combination or merging of the respective (starting) batches.

According to the invention, it is possible in particular to proceed in such a way that the components of the carrier system to be brought into contact and/or to be brought into interaction are at first divided into different batches, in particular at least two batches, and/or are introduced in different batches, in particular at least two batches, optionally in each case in combination with liquid medium preferably containing an alcohol, in particular polyol, and/or optionally followed by the addition of liquid medium preferably containing an alcohol, in particular polyol. As explained herein, the alcohol is in particular not a C₁- to C₄-monoalcohol and in particular not ethanol.

The division of the components or substances used for producing the carrier system according to the invention also makes it possible to avoid the use of the aforementioned alcohols, and this is accompanied by the aforementioned advantages.

An input of energy, in particular an input of mixing and/or stirring energy, in particular as defined above, can be performed for the respective batches, independently of each other. In addition, the respective batches can be brought to a temperature, as defined above, independently of each other. In addition, a respective batch can be present or processed in a receiving device, in particular a container and/or reactor, preferably a stirring chamber and/or stirring vessel.

In this context, the various batches, in particular the first batch and the second batch, can then be combined and/or united to form the carrier system, optionally in each case in combination with liquid medium preferably comprising an alcohol, in particular polyol, and/or optionally followed by the addition of liquid medium preferably comprising an alcohol, in particular polyol (wherein the liquid medium, as stated above, is in particular such that it does not comprise any C₁- to C₄-monoalcohol, in particular no ethanol).

In this context, it may be provided according to the invention that, in order to form the carrier system, at least one of the batches, in particular the first or the second batch, is fed and/or added to the at least one other batch and/or is combined and/or merged therewith, in particular so that a joint (merged) batch is obtained, in particular wherein an input of energy, in particular an input of mixing and/or stirring energy, in particular as defined above, is performed for the joint batch; and/or in particular wherein the joint batch is brought to a temperature as defined above; and/or in particular wherein the joint batch is present and/or processed in a receiving device, in particular a container and/or reactor, preferably a stirred chamber and/or stirred vessel.

In the context of the present invention, the following procedure has also proved to be of special advantage. Thus, according to the invention, it may be provided that the hydrophobic substances and optionally the amphiphilic substances, optionally in combination with liquid medium preferably containing an alcohol, in particular polyol, in particular as previously defined, are provided in a first batch and/or that a first batch is provided which contains the hydrophobic substances and optionally the amphiphilic substances, optionally in combination with liquid medium preferably containing an alcohol, in particular polyol, in particular as previously defined, and
in that the hydrophilic substances and optionally the amphiphilic substances, in particular in combination with liquid medium preferably containing an alcohol, in particular a polyol, in particular as defined above, are presented in a second batch and/or wherein a second batch is provided which contains the hydrophilic substances and optionally the amphiphilic substances, in particular in combination with liquid medium preferably containing a polyol, in particular as defined above, and
optionally that the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing an alcohol, in particular polyol, in particular as defined above;
wherein, in particular, the first batch and the second batch and optionally the third batch are subsequently combined and/or united to form the carrier system.

In this context, it is particularly possible to proceed in such a way that the hydrophilic substances comprise a Griffin HLB value of at least 6, preferably at least 7, preferably at least 8, and/or that the hydrophobic substances comprise a Griffin HLB value of less than 6, preferably at most 5.5, preferably at most 5, and/or that the hydrophilic substances comprise a Log P value of at most 1.11, preferably at most 0.93, preferably at most 0.80, and/or that the hydrophobic substances comprise a Log P value of more than 1.11, preferably at least 1.22, preferably at least 1.36.

The supply or introduction of the active ingredient or substance (vi) in relation to the aforementioned batches can be effected in particular depending on the underlying hydrophobic or hydrophilic properties.

According to a specific embodiment according to the invention, it may in particular be provided that the substance (i), optionally joint with at least one substance different from substance (ii), in particular in combination with a liquid medium preferably containing an alcohol, in particular a polyol, in particular as defined above, is presented in a first batch and/or that a first batch is provided which contains the substance (i), optionally joint with at least one substance different from substance (ii), in particular in combination with a liquid medium preferably containing an alcohol, in particular a polyol, in particular as defined above, and that the substance (i), optionally joint with at least one substance different from substance (ii), in particular in combination with a liquid medium preferably containing an alcohol, in particular a polyol, in particular as defined above, is presented in a second batch.

in that the substance (ii), optionally together with at least one substance different from substance (i), in particular in combination with a liquid medium preferably containing an alcohol, in particular polyol, in particular as defined above, is presented in a second batch, wherein a second batch is provided which contains the substance (ii), optionally together with at least one substance different from substance (i), in particular in combination with a liquid medium preferably containing an alcohol, in particular polyol, in particular as defined above, and
optionally wherein the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing an alcohol, in particular polyol, in particular as defined above,
wherein, in particular, the first batch and the second batch and optionally the third batch are subsequently combined and/or united to form the carrier system.

According to a further specific embodiment of the present invention, it may furthermore also be provided that the substance (i) is presented together with substance (ii), in particular with at least a part of substance (ii), optionally together with at least one substance different from substance (ii), in particular in combination with a liquid medium preferably containing an alcohol, in particular polyol, in particular as defined above, in a first batch and/or that a first batch is provided which contains substance (i), optionally together with at least one substance different from substance (ii), in particular in combination with an alcohol, in particular as defined above, in a first batch, in particular as defined above, in a first batch and/or in that a first batch is provided which contains the substance (i), optionally joint with at least one substance other than substance (ii), in particular in combination with a liquid medium preferably containing an alcohol, in particular polyol, in particular as defined above, and
in that the substance (ii), optionally joint with at least one substance different from substance (i), in particular in combination with a liquid medium preferably containing an alcohol, in particular polyol, in particular as defined above, is presented in a second batch and/or wherein a second batch is provided which contains the substance (ii), optionally joint with at least one substance different from substance (i), in particular in combination with a liquid medium preferably containing an alcohol, in particular polyol, in particular as defined above, and
optionally that the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing an alcohol, in particular polyol, in particular as defined above,
wherein, in particular, the first batch and the second batch and optionally the third batch are subsequently combined and/or united to form the carrier system.

According to a still further specific embodiment of the present invention, it may furthermore also be provided that the substance (i), in particular in combination with liquid medium preferably containing an alcohol, in particular polyol, in particular as defined above, is presented in a first batch and/or that a first batch is provided which contains the substance (i), in particular in combination with liquid medium preferably containing an alcohol, in particular polyol, in particular as defined above, wherein the first batch is brought to a temperature in the range from 10° C. to 30° C., in particular in the range from 15° C. to 25° C., and
in that the substances (ii), (iii) and (vii), optionally in combination with liquid medium preferably containing an alcohol, in particular polyol, in particular as defined above, are presented in a second batch and/or in that a second batch is provided which contains the substances (ii), (iii) and (vii), optionally in combination with liquid medium preferably containing an alcohol, in particular polyol, in particular as defined above, and in particular wherein the second batch is brought to a temperature in the range from 60° C. to 90° C., in particular in the range from 70° C. to 80° C., and
optionally that the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing an alcohol, in particular polyol, in particular as defined above;
in particular wherein the first batch and the second batch, in particular after cooling the second batch preferably to a temperature below 70° C., in particular in the range from 40° C. to 55° C., and optionally the third batch are subsequently combined and/or united to form the carrier system, in particular wherein the second batch and optionally the third batch are added to the first batch.

The supply or introduction of the active ingredient or substance (vi) with respect to the aforementioned batches can thus be effected taking into account the thermal stability of the active ingredient. In particular, the addition or supply can be effected in the first batch or a third batch, in particular if it is an active ingredient with lower thermal stability. In principle, the addition or supply can also be effected in the second batch, in particular if the active ingredient has a higher thermal stability.

According to a further special embodiment, it is also possible according to the invention that the substance (ii), in particular in combination with a liquid medium preferably containing a polyol, in particular as defined above, is provided in a first batch and/or that a first batch is provided which contains the substance (ii), in particular in combination with a liquid medium preferably containing an alcohol, in particular a polyol, in particular as defined above, wherein the first batch is brought to a temperature in the range from 15° C. to 60° C., in particular in the range from 20° C. to 50° C., and
in that the substances (i) and optionally (iii) and/or (vii), preferably the substances (i), (iii) and (vii), optionally in combination with liquid medium preferably containing an alcohol, in particular polyol, in particular as defined above, are presented in a second batch and/or wherein a second batch is provided which contains the substances (i) and optionally (iii) and/or (vii), preferably the substances (i), (iii) and (vii), optionally in combination with liquid medium preferably containing an alcohol, in particular polyol, in particular as defined above, in particular wherein the second batch is brought to a temperature in the range from 60° C. to 90° C., in particular in the range from 70° C. to 80° C., and
optionally that the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing an alcohol, in particular polyol, in particular as defined above;
in particular wherein the first batch and the second batch, in particular after cooling the second batch preferably to a temperature below 70° C., in particular in the range from 40° C. to 55° C., and optionally the third batch are subsequently combined and/or united to form the carrier system, in particular wherein the second batch and optionally the third batch are added to the first batch.

In the context of the present invention, it is completely surprising that the use of C₁- to C₄-monoalcohols or ethanol and chloroform as solvents for the stated components can be avoided. In accordance with the invention, it is also generally possible to dilute or further process the final joint batch obtained with further liquid medium, which in particular contains an alcohol, preferably a polyol.

On this basis, a ready-to-use composition can thus be obtained which contains the carrier system present in particles. As part of the method according to the invention, effective incorporation or attachment of the active ingredient into or onto the carrier system can also be effected.

In accordance with the invention, a division of the components used can also be effected on the basis of or as a function of their respective HLB values according to Griffin, wherein various batches can be used to combine the respective components, which are then combined, leading to the formation of defined compositions with defined particles of the carrier system.

With regard to the distribution of the components used according to the invention with respect to their HLB values according to Griffin or their Log P value with the corresponding distribution to different batches, the following procedure can be used in particular according to a further specific embodiment of the present invention: Thus, according to the invention, it may in particular be as follows,
in that the substances with an HLB value according to Griffin of at least 6, preferably at least 7, preferably at least 8, or with a Log P value of at most 1.11, preferably at most 0.93, preferably at most 0.80, in particular in combination with a liquid medium preferably containing an alcohol, in particular polyol, in particular as defined above, are preferred in a first batch, and/or in that a first batch is provided which contains the substances with an HLB value according to Griffin of at least 6, preferably at least 7, preferably at least 8, or with a Log P value of at most 1.11, preferably at most 0.93, preferably at most 0.80, in particular in combination with a liquid medium preferably containing an alcohol, in particular polyol, in particular as defined above, in particular wherein the first batch is brought to a temperature in the range from 15° C. to 60° C., in particular in the range from 20° C. to 50° C., and
in that the substances with an HLB value according to Griffin of less than 6, preferably at most 5.5, preferably at most 5, and/or with a Log P value of more than 1.11, preferably at least 1.22, preferably at least 1.36, optionally in combination with liquid medium preferably containing an alcohol, in particular polyol, in particular as defined above, are introduced into a second batch and/or wherein a second batch is provided, which contains the substances with an HLB value according to Griffin of less than 6, preferably at most 5.5, preferably at most 5, and/or with a Log P value of more than 1.11, preferably at least 1.22, preferably at least 1.36, optionally in combination with liquid medium preferably containing an alcohol, in particular polyol, in particular as defined above, in particular wherein the second batch is brought to a temperature in the range from 60° C. to 90° C., in particular in the range from 70° C. to 80° C., and
optionally that the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing an alcohol, in particular polyol, in particular as defined above;
in particular wherein the first batch and the second batch, in particular after cooling the second batch preferably to a temperature below 70° C., in particular in the range from 40° C. to 55° C., and optionally the third batch are subsequently combined and/or united to form the carrier system, in particular wherein the second batch and optionally the third batch are added to the first batch.

According to yet another special embodiment, it is also possible to proceed in such a way that the substances (i), (ii) and optionally (x), preferably the substances (i), (ii) and (x), in particular in combination with liquid medium preferably containing an alcohol, in particular polyol, in particular as defined above, are presented in a first batch and/or that a first batch is provided, which contains the substances (i), (ii) and optionally (x), preferably the substances (i), (ii) and (x), in particular in combination with liquid medium preferably containing an alcohol, in particular polyol, in particular as defined above, in particular wherein the first batch is brought to a temperature in the range from 30° C. to 90° C., in particular in the range from 50° C. to 80° C., and
in that the substances (iii) and/or (v), preferably the substances (iii) and (v), optionally in combination with liquid medium preferably containing an alcohol, in particular polyol, in particular as defined above, are presented in a second batch and/or wherein a second batch is provided which contains the substances (iii) and/or (v), preferably the substances (iii) and (v), optionally in combination with liquid medium preferably containing an alcohol, in particular polyol, in particular as defined above, in particular wherein the second batch is brought to a temperature in the range from 40° C. to 100° C., in particular in the range from 60° C. to 90° C., and
optionally that the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing a polyol, in particular as defined above;
in particular wherein the first batch and the second batch are subsequently combined and/or united to form the carrier system, in particular wherein the second batch is added to the first batch, in particular followed by the addition of liquid medium preferably containing an alcohol, in particular polyol, and/or the third batch.

According to a further preferred embodiment according to the invention, in particular with regard to the previously mentioned DMI according to substance (ix), it is also possible to proceed according to the invention in such a way that the substances (i), (ii) and (ix) and optionally (iii) and optionally (v), preferably the substances (i), (ii), (iii), (v) and (ix), in particular in combination with a liquid medium preferably containing an alcohol, in particular polyol, in particular as defined above, in a first batch and/or in that a first batch is provided which contains the substances (i), (ii) and (ix) and optionally (iii) and optionally (v), preferably the substances (i), (ii), (iii), (v) and (ix), in particular in combination with liquid medium preferably containing an alcohol, in particular polyol, in particular as defined above, in particular wherein the first batch is brought to a temperature in the range from 30° C. to 80° C., in particular in the range from 45° C. to 65° C., and
in that a second batch preferably comprises or is formed from a liquid medium containing an alcohol, in particular a polyol, in particular as defined above, wherein the second batch is brought to a temperature in the range from 10° C. to 30° C., in particular in the range from 15° C. to 25° C., and
optionally that the active ingredient or the substance (vi), in particular in combination with liquid medium preferably containing an alcohol, in particular polyol, in particular as defined above, is supplied and/or presented in the first batch and/or or in the second batch and/or in a third batch, preferably in a third batch, and/or optionally that a third batch is provided which contains the active ingredient or the substance (vi), in particular in combination with liquid medium preferably containing a polyol, in particular as defined above;
in particular wherein the first batch and the second batch are subsequently combined and/or united to form the carrier system, in particular wherein the second batch is added to the first batch, in particular followed by the addition of preferably an alcohol, in particular polyol, containing liquid medium and/or the third batch.

According to a further embodiment according to the invention, it is also possible to proceed in such a way that the substances (i) and (ii) and optionally (iii) and optionally (v) and optionally (vi) (i.e. the active compound), preferably the substances (i), (ii), (iii), (v) and (vi), in particular in combination with liquid medium preferably containing an alcohol, in particular polyol, in particular as defined above, are presented in a first batch and/or that a first batch is provided which contains the substances (i) and (ii) and optionally (iii) and optionally (v) and optionally (vi), preferably the substances (i), (ii), (iii), (v) and (vi), in particular in combination with liquid medium preferably containing an alcohol, in particular polyol, in particular as defined above, in particular wherein the first batch is brought to a temperature in the range from 30° C. to 80° C., in particular in the range from 45° C. to 65° C., and
in that a second batch preferably comprises or is formed from a liquid medium containing an alcohol, in particular a polyol, in particular as defined above, wherein the second batch is brought to a temperature in the range from 10° C. to 30° C., in particular in the range from 15° C. to 25° C., and
optionally that the active ingredient or the substance (vi), in particular in combination with liquid medium preferably containing an alcohol, in particular polyol, in particular as defined above, is (moreover) supplied and/or presented in the first batch and/or in the second batch and/or in a third batch, preferably in a third batch, and/or optionally that moreover a third batch is provided which contains the active ingredient or the substance (vi), in particular in combination with liquid medium preferably containing a polyol, in particular as defined above;
in particular wherein the first batch and the second batch are subsequently combined and/or united to form the carrier system, in particular wherein the second batch is added to the first batch, in particular followed by the addition of preferably an alcohol, in particular polyol, containing liquid medium and/or the third batch.

With regard to the previously mentioned batches, further batches may be provided: In particular, it may also be provided according to the invention that the active ingredient and/or the substance (iv), optionally in combination with substance (ii) and/or (iii) and/or in particular in combination with liquid medium preferably containing an alcohol, in particular polyol, is presented in a fourth batch and/or that a fourth batch is also provided, which contains the substance (iv), optionally in combination with substance (ii) and/or (iii) and/or in particular in combination with liquid medium preferably containing an alcohol, in particular polyol, wherein the fourth batch is added to at least one of the further batches and/or the previously combined further batches and/or the composition.

In addition, it may also be provided according to the invention that the substance (iv), optionally in combination with substance (ii) and/or (iii) and/or in particular in combination with liquid medium preferably containing an alcohol, in particular polyol, in particular as defined above, is presented in a fifth batch and/or that a fifth batch is also provided, which contains the substance (iv), optionally in combination with substance (ii) and/or (iii) and/or in particular in combination with liquid medium preferably containing an alcohol, in particular polyol, in particular wherein the fifth batch is added to at least one of the further batches and/or the previously combined further batches and/or the composition.

According to the invention, it has proved advantageous if the batches are combined and/or brought together in succession and/or in a cascade, in particular in the form of a continuous-cascade process operation, preferably with the use of respective receiving devices, in particular containers and/or reactors, preferably stirred chambers and/or stirred tanks, in particular wherein the receiving devices are arranged in succession and/or in a cascade.

According to the invention, however, it may also be provided that all components of the carrier system to be brought into contact and/or interaction are brought together and/or combined jointly, in particular directly and/or directly (i.e. in particular without preceding division into different batches), in particular together with liquid medium preferably containing an alcohol, in particular polyol, and/or are combined and/or united to form the carrier system, in particular followed by the (further) addition of liquid medium preferably containing an alcohol, in particular polyol, preferably in a joint batch, preferably in a joint receiving device, in particular container and/or reactor, preferably stirred chamber and/or stirred vessel, or else
that at first a liquid medium preferably containing an alcohol, in particular a polyol, optionally together with at least a part of at least one component of the carrier system, followed by the addition of at least one further batch which contains or consists of the remaining, preferably all the remaining, components of the carrier system to be brought into contact and/or interaction jointly, in particular together with liquid medium preferably containing an alcohol, in particular polyol, in particular by bringing together and/or combining all the batches to form the carrier system, in particular followed by the (further) addition of liquid medium preferably containing an alcohol, in particular polyol, preferably in a joint batch, preferably in a joint receiving device, in particular container and/or reactor, preferably stirred chamber and/or stirred vessel.

For this purpose, it may be provided that an input of energy, in particular an input of mixing and/or stirring energy, in particular as defined above, is performed for the joint batch; and/or that, preferably for the joint batch, a temperature as defined above is set.

It may also be provided, in particular, that the components of the carrier system are melted and/or converted into a liquid state before and/or during merging and/or combining and/or that the liquid medium and/or the batch containing the liquid medium is set and/or brought to a temperature of at most 40° C.

With regard to the aforementioned process operations, it is in particular the case that the liquid medium preferably containing an alcohol, in particular polyol, is a medium as defined above, in particular independently for the respective batches; or that the alcohol, in particular polyol, is an alcohol, in particular polyol, as defined above, in particular independently for the respective batches.

In this respect, it is particularly the case according to the invention wherein the alcohol is not a C₁- to C₄-monoalcohol, preferably not ethanol, or in particular wherein the alcohol does not comprise a C₁- to C₄-monoalcohol, in particular not ethanol.

In particular, different liquid media or alcohols, in particular polyols, can be used for the respective batches, in particular in coordination with the components present in this respect, in particular in accordance with the aforementioned configurations.

According to the invention, it is also possible to proceed in such a way that the carrier system obtained in accordance with the process or the carrier system obtained in accordance with the process and loaded and/or equipped with the active pharmaceutical ingredient or the composition obtained in accordance with the process, which comprises the carrier system or the carrier system loaded and/or equipped with the active pharmaceutical ingredient and the liquid medium is cooled, in particular by adding liquid (further) medium which has been adjusted and/or cooled to a temperature below the temperature of the carrier system and/or composition obtained, in particular as defined above.

In this respect, the liquid medium to be added may comprise a temperature which is at least 5° C., in particular at least 10° C., preferably at least 15° C., below the temperature of the carrier system obtained or of the carrier system loaded and/or equipped with the active pharmaceutical ingredient obtained or of the composition obtained.

In particular, the liquid medium to be added may comprise a temperature in the range from −20° C. to 40° C., in particular in the range from −10° C. to 30° C., preferably in the range from 0° C. to 25° C., preferably in the range from −5° C. to 20° C.

According to the invention, it may in particular be provided in this context that the (further) liquid medium, which is set to a temperature below the temperature of the obtained carrier system and/or the obtained composition and/or is cooled, is added at least substantially immediately and/or promptly (e.g. at most 60 s, in particular at most 30 s, preferably at most 10 s) after obtaining the carrier system and/or the composition.

The liquid medium, which in particular has been adjusted or cooled to a defined temperature, can thus be added to the carrier system or composition. The resulting cooling further stabilizes the carrier system or the relevant active ingredient or composition. The cooling effect ensures further thermostabilization of the carrier system or the active ingredient or composition.

In accordance with the invention, the method is particularly such that the in particular continuous method and/or process control (production control), in particular online and/or inline process control, as described above, is used to monitor the method status and/or method progress, preferably to monitor and/or set the formation of the particles of the carrier system, in particular lipid nanoparticles (LNP), and/or in particular for monitoring and/or setting process parameters, such as input of energy, preferably input of mixing and/or stirring energy, input and/or removal of heat energy, flow behavior, mass throughput, volume flows or the like, in particular; in particular wherein the method and/or process control is effected by acquiring at least one process parameter, in particular at least two process parameters, in particular wherein the process parameter is selected from the group of temperature, conductivity, viscosity and pH value, preferably conductivity, in particular in each case of the liquid medium, preferably together with the constituents and/or substances introduced therein, or the composition and/or the respective batches, in particular as defined above, and combinations thereof.

According to the invention, the in particular continuous method and/or process control (production control) can be effected by acquiring the conductivity, in particular of the liquid medium, preferably together with the components and/or substances introduced therein, or the composition and/or the respective batches, in particular as defined hereinbefore, optionally in combination with at least one further process parameter, in particular as defined hereinbefore. In particular, the conductivity, in particular the conductivity of the liquid medium, preferably together with the constituents and/or substances introduced therein, or the conductivity of the composition and/or the conductivity of the respective batches, in particular as defined above, can be selected as the at least one process parameter, optionally wherein the method and/or process control is effected via the additional acquiring of at least one further process parameter, in particular as defined above. According to the invention, the preferably selected process parameter thus represents the conductivity as defined above.

With regard to the process parameters to be acquiring. (relative) changes in this respect, for example (relative) conductivity changes, can be used to control or adjust process parameters such as input of energy or the like. This enables effective process control or process operation, which can also be automated

As part of the control or monitoring of the process operation according to the invention, it is in particular possible to carry out the production of the underlying particles under computer control. As far as the conductivity is concerned, this can in particular be used as a measure with regard to a correlation to the particle concentration or production. Similarly, there is a correlation to the electrolyte concentration, for example. Dilution, for example, can also be set in this way. The viscosity as a characteristic of gel phases in particular can also be used with regard to the formation of particles. The viscosity can, for example, be determined directly via the torque of the stirrer, which is similar to the operation of a Brookfield viscometer. In particular, the torque required to maintain the movement can be measured, from which the stress and thus the viscosity can then be derived. In particular, the viscosity can be determined using Brookfield or as Brookfield viscosity. Furthermore, in the case of viscoelastic behaviour, this can also be characterized by the loss factor tan δ. In this context, viscoelastic behaviour is characterized in particular by tan δ<1. In general, the viscoelastic behavior decreases with increasing temperature. At the phase transition temperature, a value tan δ>1 is assumed.

In particular, the viscosity can be measured in accordance with EN ISO 3219, especially ISO 3219-1:2021 or EN ISO 3219-1:2021. A viscometer can also be used for this purpose. The particle size distribution can also be determined. For example, methods based on light scattering or laser diffractometry can be used.

The method according to the invention can in particular be carried out in an automated, preferably at least essentially fully automated, but at least partially automated, or computer-controlled manner. As part of the method according to the invention, an overall composition based on particles of the carrier system present in a liquid medium with a corresponding finish or loading based on an active ingredient can be provided, which comprises overall defined properties.

In particular, after formation of the carrier system, a preferably disperse composition, in particular a dispersion, preferably as defined above, which comprises the carrier system or the carrier system loaded and/or equipped with the active pharmaceutical ingredient and the liquid medium, can result and/or in particular wherein after formation of the carrier system a composition, in particular as defined above, results in the form of a dispersion of the carrier system or the carrier system loaded and/or equipped with an active pharmaceutical ingredient in the liquid medium.

The composition obtainable by the process may comprise the carrier system, preferably the particles, preferably the lipid nanoparticles, in an amount in the range from 5 wt. % to 95 wt. %, in particular in the range from 10 wt. % to 90 wt. %, preferably in the range from 15 wt. % to 85 wt. %, preferably in the range from 20 wt. % to 80 wt. %, particularly preferred in the range from 25 wt. % to 75 wt. %, based on the composition.

In particular, the carrier system may be present in the composition in particulate form, in particular in the form of lipid nanoparticles (LNP), preferably lipid nanoparticles (LNP) in the form of micelles, vesicles and/or discoidal (disk-shaped) lipid nanoparticles (LNP) (disk lipid nanoparticles or disk LNP), preferably discoidal (disk-shaped) lipid nanoparticles (LNP), in particular as defined above.

The composition produced on the basis of the method according to the invention is characterized in particular by a low water activity. In particular, the composition may comprise a water activity of less than 1, in particular at most 0.9, preferably at most 0.7, preferably at most 0.5, particularly preferred at most 0.3.

In general, the composition may comprise a water activity in the range from 0 to 1, in particular in the range from 0 to 0.9, preferably in the range from 0 to 0.7, preferably in the range from 0 to 0.5, particularly preferred in the range from 0 to 0.3.

The water activity can be determined in particular according to ISO 21807:2004 and/or according to ISO 18787:2017 and/or 21 CFR Part 11, in particular as in ISO 29621:2017. The water activity (also referred to as a_{w} value or activity of water) of a composition is a measure of the available or active water, which may differ from the mere indication of the water content. The water activity influences, among other things, the growth of microorganisms, the course of chemical processes such as fat oxidation or oxidation processes in general and also the physicochemical properties of the compositions. The water activity is defined in particular as the ratio of the water vapor partial pressure (p) of a composition to the saturation vapor pressure of pure water (p₀) and can be determined in particular using the formula a_{w}=p/p₀ (for example at room temperature or 20° C.). In particular, the compositions according to the invention may behave in such a way—without wishing to limit or refer to this theory—that water is partially present in bound form, which leads to reduced water activity. In addition to the above configurations, the water activity can also be determined in accordance with the ISO 29621 standard, in particular with regard to a so-called preservation stress test.

The compositions according to the invention thus comprise relatively low water activities overall. This also results in a high storage stability or a high oxidation protection with respect to the underlying components or ingredients.

With the method according to the invention, it is also possible, in particular, to provide carrier systems or compositions with defined or customized Log P values in a targeted manner. This can, for example, increase the stability of the corresponding composition or the bioavailability of the carrier system or the active ingredient.

Thus, the carrier system and/or the composition, in particular the carrier system, may comprise a Log P value, in particular total Log P value, of at most 5, in particular at most 4, preferably at most 3, particularly preferred at most 2, further preferred at most 1. In this context, the carrier system and/or the composition, in particular the carrier system, may comprise a Log P value, in particular total Log P value, in the range from 0.1 to 5, in particular in the range from 0.2 to 4, preferably in the range from 0.3 to 3, particularly preferred in the range from 0.4 to 2.

In particular, the components and/or substances of the carrier system, in particular substances (i), (ii), (iii), (iv) and/or (v), may be selected with the proviso that the resulting carrier system comprises a Log P value, in particular a total Log P value, of at most 5, in particular at most 4, preferably at most 3, particularly preferred at most 2, further preferably at most 1. In addition, the components and/or substances of the carrier system, in particular the substances (i), (ii), (iii), (iv) and/or (v), can be selected with the proviso that the resulting carrier system comprises a Log P value, in particular a total Log P value, in the range from 0.1 to 5, in particular in the range from 0.2 to 4, preferably in the range from 0.3 to 3, particularly preferred in the range from 0.4 to 2.

Overall, the aforementioned total Log P values for the carrier system also lead to optimized incorporation or attachment of active ingredients, such as mRNA. The carrier systems provided according to the invention, in particular in the form of lipid nanoparticles and in particular disc-shaped lipid nanoparticles, are also characterized in particular by the fact that a high bioavailability of the active ingredient or the particles as such is also present from the fact that relatively low Log P values are present in large (surface) areas of the carrier system or the particles and in this context the so-called Lipinski's rule of five is also fulfilled. It can be ensured that an overall Log P value is also present for the entire (surface) area of the carrier systems or disk-shaped lipid nanoparticles provided according to the invention, which fulfills the rule of five according to Lipinski.

As the particle size of the lipid nanoparticles decreases, it can also behave in particular in such a way that greater contact is formed between the cell and the lipid nanoparticle, wherein the average contact area per cell increases. This also leads to a high release of active ingredients. Overall, this also enables high bioavailability. In this context, optimal hydrophilicity or lipophilicity is also present in relation to the carrier system according to the invention. It should also be noted that active ingredients with an overall hydrophilicity or lipophilicity that is too high generally comprise an inadequate or non-optimal bioavailability, for example because there are no optimal permeability properties or the like. In this context, reference can be made to the so-called rule of five according to Lipinski, which states that the Log P value for a drug similarity or active ingredients should generally not exceed the value of five. In addition, based on the defined total Log P values, a stable composition with the carrier systems according to the invention is present. In particular, there is also a high compatibility with the underlying medium, also with regard to the additional use of polyol or the like in the medium, as previously mentioned. Based on the special HLD or (total) Log P values, the components used are also optimally compatible, resulting in optimized carrier systems. This means that tailor-made carrier systems with high compatibility with the underlying medium can also be provided on this basis.

According to the invention, it is possible for the HLB values and/or the Log P values to relate to a temperature, in particular process temperature, preferably mixing temperature, below the phase inversion temperature of the substance (ii) used and/or below the phase inversion temperature of the composition based on the substance (ii) used, or for the method to be carried out below the phase inversion temperature of the substance (ii) used and/or below the phase inversion temperature of the composition based on the substance (ii) used. In particular, the method and/or process control, in particular monitoring, can be effected by acquiring the conductivity and/or the surface tension, in particular dynamic surface tension, in particular in each case of the liquid medium, preferably together with the components and/or substances introduced therein, and/or of the composition. This can also be used to provide defined carrier systems or compositions.

The control or prevention of a phase inversion can, for example, be effected by acquiring process parameters, for example by acquiring or measuring the conductivity of the underlying medium, which in particular comprises the components or substances for forming the carrier system or the carrier system as such. In this respect, the surface tension, in particular dynamic surface tension, of the underlying medium or system can also be used as a process parameter.

According to the invention, the method according to the invention is generally carried out below the cloud point and/or below the demixing point of the underlying system and/or the liquid medium, preferably together with the components and/or substances introduced therein, and/or the composition. On this basis, overall optimized and uniform carrier systems can be obtained.

According to the present aspect, the present invention also relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
   in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results;
in particular wherein the substances are at first at least partially divided into different batches or are presented in different batches and are then combined and/or united to form the carrier system, preferably wherein the distribution of the substances to the different batches and/or the presentation of the substances in different batches is effected as a function of their respective hydrophilicity and/or lipophilicity and/or as a function of their HLB values according to Griffin and/or as a function of their Log P values, in particular wherein the distribution is effected in such a way that corresponding substances, in particular substances with the same or similar hydrophilicity and/or lipophilicity and/or substances with the same or similar Log P values, are each presented in the same batch, substances with the same or similar HLB values according to Griffin and/or with the same or similar Log P values, are each presented in the same batch and/or in particular wherein similar substances, in particular substances with the same or similar hydrophilicity and/or lipophilicity and/or substances with the same or similar HLB values according to Griffin and/or with the same or similar Log P values, are each presented in the same batch.

According to the present aspect, the present invention also relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
(ii) at least one non-ionic amphiphilic and/or surfactant substance, in particular selected from the group of polyglycerol esters, alkylpolyglycosides, amphiphilic and/or surfactant PEGylated (polyoxyethylated) substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids as well as combinations and mixtures thereof, even more preferably selected from PEGylated fatty acids and salts and esters thereof, in particular polyol esters of PEGylated fatty acids;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
(iv) optionally at least one cationic lipid, in particular an amine-based cationic lipid, preferably a cationic lipid based on a tertiary amine;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
wherein the method is characterized by at least one of the following features and/or measures (1a) to (5a), in particular by a combination of at least two, preferably at least three, preferably at least four, particularly preferred all five, of the following features and/or measures (1a) to (5a):
(1a) the substance (i) comprises an HLB value according to Griffin of at most 11, in particular at most 8, in particular in the range from 0.5 to 8, preferably in the range from 2 to 7, preferably in the range from 3 to 6, and/or the substance (i) comprises a Log P value of at least 0.55, in particular at least 0.80, in particular in the range from 0.80 to 18.97, preferably in the range from 0.93 to 3.88, preferably in the range from 1.11 to 2.44;
(2a) the substance (ii) comprises an HLB value according to Griffin of at least 13, in particular in the range from 13 to 19, preferably in the range from 13 to 17.9, preferably in the range from 13.5 to 17.5, particularly preferred in the range from 14 to 17, and/or the substance (ii) comprises a Log P value of at most 0.46, in particular in the range from 0.32 to 0.46, preferably in the range from 0.33 to 0.44, preferably in the range from 0.34 to 0.42;
(3a) the substance (iii) comprises an HLB value according to Griffin of at most 1.5, in particular in the range from 0.5 to 1.5, preferably in the range from 0.6 to 1.4, preferably in the range from 0.7 to 1.3, and/or the substance (iii) comprises a Log P value of at least 5.40, in particular in the range from 5.40 to 18.97, preferably in the range from 5.84 to 15.40, preferably in the range from 6.36 to 12.91;
(4a) the substance (iv) comprises an HLB value according to Griffin of at most 4, in particular in the range from 1 to 4, preferably in the range from 1.5 to 3.5, preferably in the range from 2 to 3, and/or the substance (iv) comprises a Log P value of at least 1.76, in particular in the range from 1.76 to 8.58, preferably in the range from 2.05 to 5.40, preferably in the range from 2.44 to 3.88;
(5a) the substance (v) comprises an HLB value according to Griffin of at most 8, in particular in the range from 1 to 8, preferably in the range from 1.25 to 7, preferably in the range from 1.5 to 6.5, and/or the substance (v) comprises a Log P value of at least 0.80, in particular in the range from 0.80 to 8.58, preferably in the range from 0.93 to 6.65, preferably in the range from 1.01 to 5.40;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results;
in particular wherein the substances are at first at least partially divided into different batches or are presented in different batches and are then combined and/or united to form the carrier system, preferably wherein the distribution of the substances to the different batches and/or the presentation of the substances in different batches is effected as a function of their respective hydrophilicity and/or lipophilicity and/or as a function of their HLB values according to Griffin and/or as a function of their Log P values, in particular wherein the distribution is effected in such a way that corresponding substances, in particular substances with the same or similar hydrophilicity and/or lipophilicity and/or substances with the same or similar Log P values, are each presented in the same batch, substances with the same or similar HLB values according to Griffin and/or with the same or similar Log P values, are each presented in the same batch and/or in particular wherein similar substances, in particular substances with the same or similar hydrophilicity and/or lipophilicity and/or substances with the same or similar HLB values according to Griffin and/or with the same or similar Log P values, are each presented in the same batch.

According to the aforementioned methods, it is in particular equally the case that the substances with an HLB value according to Griffin of at least 6, preferably at least 7, preferably at least 8, or with a Log P value of at most 1.11, preferably at most 0.93, preferably at most 0.80, in particular in combination with a liquid medium preferably containing a polyol, in particular as defined above, are provided in a first batch and/or in particular wherein a first batch is provided which contains the substances with an HLB value according to Griffin of at least 6, preferably at least 7, preferably at least 8, and/or with a Log P value of at most 1.11, preferably at most 0.93, preferably at most 0.80, in particular in combination with a liquid medium preferably containing a polyol, in particular as defined above, and/or in particular wherein the substances with an HLB value according to Griffin of less than 6, preferably at most 5.5, preferably at most 5, and/or with a Log P value of more than 1.11, preferably at least 1.22, preferably at least 1.36, optionally in combination with preferably a polyol-containing liquid medium, in particular as defined above, in a second batch and/or in particular wherein a second batch is provided which contains the substances with an HLB value according to Griffin of less than 6, preferably at most 5.5, preferably at most 5, and/or with a Log P value of more than 1.11, preferably at least 1.22, preferably at least 1.36, optionally in combination with preferably a polyol-containing liquid medium, in particular as defined above.

In this regard, the aforementioned methods according to the present aspect can also be carried out in the at least substantially complete absence of aliphatic monoalcohols, in particular in the at least substantially complete absence of aliphatic C1- to C4-monoalcohols, preferably in the at least substantially complete absence of ethanol.

Through the targeted use and combination of the substances or components of the carrier system or the medium, the formation of the carrier system can be further controlled or customized. Due to the special HLB values or Log P values of the components or substances used for the carrier system, there is also a high degree of compatibility and coordination of the components, so that defined carrier systems can also be provided on this basis in a targeted and purpose-oriented manner. For example, the use of a polyol can further influence or control the formation of aggregates.

According to the present aspect, the present invention further relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the non-ionic amphiphilic and/or surfactant substance comprises a Log P value of at most 0.46;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
   in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results;
wherein the liquid medium comprises water and at least one alcohol;
wherein the alcohol is selected from the group consisting of monohydric alcohols, polyhydric alcohols, in particular polyols, and mixtures and combinations thereof; and/or
in particular wherein the alcohol is a polyhydric alcohol, in particular a polyol; and/or
in particular wherein the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof; and/or
wherein the alcohol, in particular the polyol, is selected from the group consisting of propylene glycol, polyethylene glycol, sorbitol, mannitol, glycerol, sucrose, panthenol and mixtures thereof and combinations thereof; and/or
in particular wherein the alcohol, in particular the polyol, is glycerol; and/or
in particular wherein the alcohol, in particular the polyol, is panthenol; and/or
in particular wherein the alcohol is not a C₁- to C₄-monoalcohol, preferably not an ethanol, and/or in particular wherein the alcohol does not comprise a C₁- to C₄-monoalcohol, in particular not an ethanol; and/or
in particular wherein the liquid medium does not comprise any C₁- to C₄-monoalcohol, in particular no ethanol.

According to the present aspect, the present invention also relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
   in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results;
wherein the liquid medium comprises water and at least one alcohol and
wherein the liquid medium comprises or consists of a mixture of water and at least one alcohol, in particular polyol, wherein the weight-related quantitative ratio of water to alcohol, in particular polyol, [water:alcohol, in particular polyol] is in the range from 200:1 to 1:50, in particular in the range from 100:1 to 1:20, preferably in the range from 50:1 to 1:5, preferably in the range from 20:1 to 1:1, in particular with respect to the liquid medium, and/or is set;
wherein the alcohol is selected from the group consisting of monohydric alcohols, polyhydric alcohols, in particular polyols, and mixtures and combinations thereof; and/or
in particular wherein the alcohol is a polyhydric alcohol, in particular a polyol; and/or
wherein the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof; and/or
wherein the alcohol, in particular the polyol, is selected from the group consisting of propylene glycol, polyethylene glycol, sorbitol, mannitol, glycerol, sucrose, panthenol and mixtures thereof and combinations thereof; and/or
in particular wherein the alcohol, in particular the polyol, is glycerol; and/or
in particular wherein the alcohol, in particular the polyol, is panthenol; and/or
in particular wherein the alcohol is not a C₁- to C₄-monoalcohol, preferably not an ethanol, and/or in particular wherein the alcohol does not comprise a C₁- to C₄-monoalcohol, in particular not an ethanol; and/or
in particular wherein the liquid medium does not comprise any C₁- to C₄-monoalcohol, in particular no ethanol.

According to the present aspect, the present invention also relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
   in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results;
wherein the liquid medium comprises water and at least one alcohol and
wherein the liquid medium is used in an amount such that the weight-related quantity ratio of carrier system, in particular the entirety of the components of the carrier system, to liquid medium [carrier system, in particular the entirety of the components of the carrier system:liquid medium] is in the range from 1:20 to 50:1, in particular in the range from 1:10 to 20:1, preferably in the range from 1:5 to 10:1, preferably in the range from 1:4 to 5:1, and/or wherein the liquid medium comprises or consists of a mixture of water and at least one alcohol, in particular polyol, wherein the weight-related quantitative ratio of water to alcohol, in particular polyol, [water:alcohol, in particular polyol] is and/or is set in the range from 200:1 to 1:50, in particular in the range from 100:1 to 1:20, preferably in the range from 50:1 to 1:5, preferably in the range from 20:1 to 1:1, in particular with respect to the liquid medium;
wherein the alcohol is selected from the group consisting of monohydric alcohols, polyhydric alcohols, in particular polyols, and mixtures and combinations thereof; and/or
in particular wherein the alcohol is a polyhydric alcohol, in particular a polyol; and/or
in particular wherein the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof; and/or
wherein the alcohol, in particular the polyol, is selected from the group consisting of propylene glycol, polyethylene glycol, sorbitol, mannitol, glycerol, sucrose, panthenol and mixtures thereof and combinations thereof; and/or
in particular wherein the alcohol, in particular the polyol, is glycerol; and/or
in particular wherein the alcohol, in particular the polyol, is panthenol; and/or
in particular wherein the alcohol is not a C₁- to C₄-monoalcohol, preferably not an ethanol, and/or in particular wherein the alcohol does not comprise a C₁- to C₄-monoalcohol, in particular not an ethanol; and/or
in particular wherein the liquid medium does not comprise any C₁- to C₄-monoalcohol, in particular no ethanol.

According to the present aspect, the present invention further relates also to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
   in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
(vi) at least one active substance, in particular as defined in one of the claims 23 to 25, in particular wherein the active substance is a therapeutic and/or prophylactic active substance, in particular therapeutic and/or prophylactic pharmaceutical active substance, and/or in particular wherein the active substance is selected from the group of vaccine active substances (vaccination active substances), antibiotic active substances antifungal active ingredients, antiviral active ingredients, antiphlogistic active ingredients, antipyretic active ingredients, antiallergic active ingredients, anticancer active ingredients, hormone active ingredients, anticoagulant active ingredients, antitumor active ingredients (such as cytostatics), nutritional supplements, vitamins, minerals, UV-absorbing substances and mixtures thereof and combinations thereof, preferably vaccine active ingredients (vaccine active ingredients);
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results;
wherein the liquid medium comprises water and at least one alcohol;
wherein the alcohol is selected from the group consisting of monohydric alcohols, polyhydric alcohols, in particular polyols, and mixtures and combinations thereof; and/or
in particular wherein the alcohol is a polyhydric alcohol, in particular a polyol; and/or
wherein the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof; and/or
wherein the alcohol, in particular the polyol, is selected from the group consisting of propylene glycol, polyethylene glycol, sorbitol, mannitol, glycerol, sucrose, panthenol and mixtures thereof and combinations thereof; and/or
in particular wherein the alcohol, in particular the polyol, is glycerol; and/or
in particular wherein the alcohol, in particular the polyol, is panthenol; and/or
in particular wherein the alcohol is not a C₁- to C₄-monoalcohol, preferably not an ethanol, and/or in particular wherein the alcohol does not comprise a C₁- to C₄-monoalcohol, in particular not an ethanol; and/or
in particular wherein the liquid medium does not comprise any C₁- to C₄-monoalcohol, in particular no ethanol.

In accordance with the present aspect, the present invention again further relates to the method for producing a preferably lipid-based carrier system, preferably in particulate form, preferably in the form of lipid nanoparticles (LNP), in particular for an active pharmaceutical ingredient; and/or for producing a preferably lipid-based carrier system loaded and/or equipped with a pharmaceutical active ingredient, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one sterol (sterol), in particular selected from the group of zoosterols, phytosterols and mycosterols as well as combinations and mixtures thereof, preferably cholesterol and/or phytosterol;
   in particular wherein the sterol comprises an HLB value according to Griffin of at most 1.5 and/or in particular wherein the sterol comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the weight-related quantitative ratio of substance (i) to substance (ii) [substance (i):substance (ii)] is in the range from 50:1 to 1:160, in particular in the range from 20:1 to 1:75, preferably in the range from 10:1 to 1:56, preferably in the range from 5:1 to 1:43, and/or is set to the aforementioned values; and/or
wherein the quantitative ratio by weight of substance (i) to substance (ii) to substance (iii) to substance (iv) to substance (v) [substance (i):substance (ii):Substance (iii):Substance (iv):substance (v)] in the range from (0.5 to 50):(1 to 80):(0.2 to 40):(1 to 80):(2 to 85), in particular (1 to 40):(2 to 75):(0.5 to 25):(2 to 75):(4 to 80), preferably (1.25 to 30):(3 to 70):(1 to 20):(3 to 70):(6 to 75), preferably (1.5 to 25):(5 to 65):(3 to 15):(5 to 65):(8 to 70), and/or is set to the aforementioned values;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results.

According to the present aspect, the present invention also relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one sterol (sterol), in particular selected from the group of zoosterols, phytosterols and mycosterols as well as combinations and mixtures thereof, preferably cholesterol and/or phytosterol;
   in particular wherein the sterol comprises an HLB value according to Griffin of at most 1.5 and/or in particular wherein the sterol comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the active ingredient, in particular as substance (vi), is a component of the carrier system and/or forms a component of the carrier system and/or wherein the carrier system comprises the active ingredient, in particular as substance (vi);
wherein the weight-related quantitative ratio of substance (i) to substance (ii) to active ingredient or substance (vi) [substance (i):substance (ii):substance (vi)] is in the range from (0.5 to 50):(1 to 80):(0.01 to 80), in particular in the range from (1 to 40):(2 to 75):(0.1 to 70), preferably in the range of (1.25 to 30):(3 to 70):(0.5 to 60), preferably in the range of (1.5 to 25):(5 to 65):(1 to 50) and/or is set to the aforementioned values; and/or
in particular wherein the weight-related quantitative ratio of substance (i) to substance (ii) to substance (iii) to substance (iv) to substance (v) to active ingredient or substance (vi) [substance (i):Substance (ii):Substance (iii):Substance (iv):Substance (v):Substance (vi)] in the range from (0.5 to 50):(1 to 80):(0.2 to 40):(1 to 80):(2 to 85):(0.01 to 80), in particular in the range from (1 to 40):(2 to 75):(0.5 to 25):(2 to 75):(4 to 80):(0.1 to 70), preferably in the range of (1.25 to 30):(3 to 70):(1 to 20):(3 to 70):(6 to 75):(0.5 to 60), preferably in the range of (1.5 to 25):(5 to 65):(3 to 15):(5 to 65):(8 to 70):(1:50), and/or is set to the aforementioned values; and/or
in particular wherein the carrier system comprises the active ingredient and/or the substance (vi) in an amount in the range from 0.01 wt. % to 80 wt. %, in particular in the range from 0.1 wt. % to 70 wt. %, preferably in the range from 0.5 wt. % to 60 wt. %, preferably in the range from 1 wt. % to 50 wt. %, based on the carrier system and/or based on the entirety of the components of the carrier system;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results.

According to the present aspect, the present invention also relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one sterol (sterol), in particular selected from the group of zoosterols, phytosterols and mycosterols as well as combinations and mixtures thereof, preferably cholesterol and/or phytosterol;
   in particular wherein the sterol comprises an HLB value according to Griffin of at most 1.5 and/or in particular wherein the sterol comprises a Log P value of at least 5.40;
(iv) optionally at least one cationic lipid, in particular an amine-based cationic lipid, preferably a cationic lipid based on a tertiary amine;
in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the weight-related quantitative ratio of substance (i) to substance (ii) [substance (i):substance (ii)] is in the range from 50:1 to 1:160, in particular in the range from 20:1 to 1:75, preferably in the range from 10:1 to 1:56, preferably in the range from 5:1 to 1:43, and/or is set to the aforementioned values; and/or
wherein the quantitative ratio by weight of substance (i) to substance (ii) to substance (iii) to substance (iv) to substance (v) [substance (i):substance (ii):Substance (iii):Substance (iv):substance (v)] in the range from (0.5 to 50):(1 to 80):(0.2 to 40):(1 to 80):(2 to 85), in particular (1 to 40):(2 to 75):(0.5 to 25):(2 to 75):(4 to 80), preferably (1.25 to 30):(3 to 70):(1 to 20):(3 to 70):(6 to 75), preferably (1.5 to 25):(5 to 65):(3 to 15):(5 to 65):(8 to 70), and/or is set to the aforementioned values;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results;
wherein the liquid medium comprises water and at least one alcohol and
wherein the liquid medium is used in an amount such that the weight-related quantity ratio of carrier system, in particular the entirety of the components of the carrier system, to liquid medium [carrier system, in particular the entirety of the components of the carrier system:liquid medium] is in the range from 1:20 to 50:1, in particular in the range from 1:10 to 20:1, preferably in the range from 1:5 to 10:1, preferably in the range from 1:4 to 5:1, and/or wherein the liquid medium comprises or consists of a mixture of water and at least one alcohol, in particular polyol, wherein the weight-related quantitative ratio of water to alcohol, in particular polyol, [water:alcohol, in particular polyol] is and/or is set in the range from 200:1 to 1:50, in particular in the range from 100:1 to 1:20, preferably in the range from 50:1 to 1:5, preferably in the range from 20:1 to 1:1, in particular with respect to the liquid medium.

According to the present aspect, the present invention further also relates to the method for producing a preferably lipid-based carrier system, preferably in particulate form, preferably in the form of lipid nanoparticles (LNP), in particular for a pharmaceutically active agent; and/or for producing a preferably lipid-based carrier system loaded and/or equipped with a pharmaceutically active agent, preferably in particulate form, preferably in the form of lipid nanoparticles (LNP), in particular methods as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one sterol (sterol), in particular selected from the group of zoosterols, phytosterols and mycosterols as well as combinations and mixtures thereof, preferably cholesterol and/or phytosterol;
   in particular wherein the sterol comprises an HLB value according to Griffin of at most 1.5 and/or in particular wherein the sterol comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant), and, after formation of the carrier system, a preferably disperse composition, in particular a dispersion which comprises the carrier system or the carrier system loaded and/or equipped with the active pharmaceutical ingredient and the liquid medium, results or, after formation of the carrier system, a dispersion of the carrier system or the carrier system loaded and/or equipped with the active pharmaceutical ingredient and the liquid medium, respectively, the carrier system loaded and/or equipped with the active pharmaceutical ingredient and the liquid medium, results or, after formation of the carrier system, a dispersion of the carrier system or of the carrier system loaded and/or equipped with an active pharmaceutical ingredient in the liquid medium results,
wherein the carrier system obtained or the carrier system loaded and/or equipped with the active pharmaceutical ingredient obtained or the composition obtained, which comprises the carrier system or the carrier system loaded and/or equipped with the active pharmaceutical ingredient and the liquid medium, is cooled, in particular by (further) addition of, in particular, cooled liquid medium, in particular as defined above,
in particular wherein the liquid medium to be added comprises a temperature which is at least 5° C., in particular at least 10° C., preferably at least 15° C., below the temperature of the carrier system obtained or of the carrier system loaded and/or equipped with the active pharmaceutical ingredient obtained or of the composition obtained, and/or
in particular wherein the liquid medium to be added comprises a temperature in the range from −20° C. to 40° C., in particular in the range from −10° C. to 30° C., preferably in the range from 0° C. to 25° C., preferably in the range from −5° C. to 20° C.

In particular, the aforementioned methods according to the present aspect of the invention may also behave in such a manner, that the liquid medium comprises water and at least one alcohol;
in that or, in particular, wherein the alcohol is selected from the group of monohydric alcohols, polyhydric alcohols, in particular polyols, and mixtures and combinations thereof; and/or
in that or in particular wherein the alcohol is a polyhydric alcohol, in particular polyol; and/or
in that or, in particular, wherein the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof; and/or
in that or in particular wherein the alcohol, in particular the polyol, is selected from the group consisting of propylene glycol, polyethylene glycol, sorbitol, mannitol, glycerol, sucrose, panthenol and mixtures thereof and combinations thereof; and/or
that or in particular wherein the alcohol, in particular the polyol, is glycerol; and/or
that or in particular wherein the alcohol, in particular the polyol, is panthenol; and/or
in that or in particular wherein the alcohol is not a C₁- to C₄-monoalcohol, preferably not an ethanol, and/or in particular wherein the alcohol does not comprise a C₁- to C₄-monoalcohol, in particular not an ethanol; and/or
in that or in particular wherein the liquid medium does not comprise any C₁- to C₄-monoalcohol, in particular no ethanol.

According to the present aspect, the present invention further relates to the method for producing a preferably lipid-based carrier system, preferably in particulate form, preferably in the form of lipid nanoparticles (LNP), in particular for a pharmaceutical active ingredient; and/or for producing a preferably lipid-based carrier system loaded and/or equipped with a pharmaceutical active ingredient, preferably in particulate form, preferably in the form of lipid nanoparticles (LNP), in particular the method as defined above,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine (lecithin);
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
   in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
wherein the method, in particular producing the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1.013.25 hPa), preferably in the form of a dispersion medium (dispersant), and, after formation of the carrier system, a preferably disperse composition, in particular a dispersion comprising the carrier system or the carrier system loaded and/or equipped with the active pharmaceutical ingredient and the liquid medium, results or, after formation of the carrier system, a dispersion of the carrier system or the carrier system loaded and/or equipped with an active pharmaceutical ingredient in the liquid medium results.

According to the present aspect, the present invention also relates to the method for producing a preferably lipid-based carrier system, preferably in particulate form, preferably in the form of lipid nanoparticles (LNP), in particular for a pharmaceutical active ingredient; and/or for producing a preferably lipid-based carrier system loaded and/or equipped with a pharmaceutical active ingredient, preferably in particulate form, preferably in the form of lipid nanoparticles (LNP), in particular method according to one of the claims,
wherein the components of the carrier system are brought into contact (with each other) and/or are brought into interaction (with each other), in particular are brought into interaction physically and/or chemically, in such a way that agglomeration, in particular aggregation, of the components is effected to form the carrier system, preferably in particulate form, preferably in the form of lipid nanoparticles (LNP),
wherein the components of the carrier system comprise the following substances:
(i) at least one phospholipid, preferably phosphatidylcholine (lecithin);
(ii) at least one non-ionic amphiphilic and/or surfactant substance, in particular selected from the group of polyglycerol esters, alkyl polyglycosides, amphiphilic and/or surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
(iv) optionally at least one cationic lipid, in particular an amine-based cationic lipid, preferably a cationic lipid based on a tertiary amine;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol.

In this context, it may be envisaged that the method, in particular the producing of the carrier system or the carrier system loaded and/or equipped with the active pharmaceutical ingredient, is carried out in a liquid medium, in particular in a medium which is liquid at a temperature in the range from 20° C. to 40° C., preferably room temperature (20° C.), and ambient pressure (1.013.25 hPa), preferably in the form of a dispersion medium (dispersant), and, after formation of the carrier system, a preferably disperse composition, in particular a dispersion comprising the carrier system or the carrier system loaded and/or equipped with the active pharmaceutical ingredient and the liquid medium, results or, after formation of the carrier system, a dispersion of the carrier system or the carrier system loaded and/or equipped with an active pharmaceutical ingredient in the liquid medium results.

It may be provided for the aforementioned further subject-matters of the present aspect,
in that the agglomeration, in particular aggregation, of the components to form the carrier system is carried out with the introduction of energy, preferably introduction of mixing and/or stirring energy, in particular into the liquid medium comprising at least some of the components and/or substances of the carrier system and/or into a mixture comprising at least some of the components and/or substances of the carrier system, wherein the energy introduced, in particular mixing and/or stirring energy, is set in such a way that an at least substantially laminar flow is present, in particular in the liquid medium comprising at least some of the components and/or substances of the carrier system and/or in the mixture comprising at least some of the components and/or substances of the carrier system, in particular with at least substantially complete avoidance of the formation of a non-laminar and/or turbulent flow and/or in particular with at least substantially complete avoidance of the formation of non-laminar and/or turbulent flow components; and/or
that a method and/or process control, in particular continuous method and/or process control (production control), in particular online and/or inline process control, is carried out, in particular for monitoring the method status and/or method progress, preferably for monitoring and/or setting the formation of the particles of the carrier system, in particular lipid nanoparticles (LNP), and/or in particular for monitoring and/or setting method parameters, such as input of energy, preferably input of mixing and/or stirring energy, input and/or removal of heat energy, flow behavior, mass throughput, volume flows or the like; wherein the method and/or process control is effected by acquiring at least one process parameter, in particular at least two process parameters, the process parameter being selected from the group of temperature, conductivity, viscosity, pH value, electrical resistance and surface tension, in particular dynamic surface tension, preferably from the group of temperature, conductivity, viscosity and pH value, in particular in each case of the liquid medium, preferably together with the constituents and/or substances introduced therein, and/or the composition and/or the respective batches and combinations thereof.

For further configurations of the method according to the invention according to the present aspect, reference can also be made to the configurations of the further aspects according to the invention, which apply accordingly in the present case.

Furthermore, according to a further aspect of the present invention, the subject-matter of the present invention is also the carrier system, preferably lipid-based, preferably in particulate form, preferably in the form of lipid nanoparticles (LNP), in particular for a pharmaceutical active ingredient, and/or preferably lipid-based carrier system loaded and/or equipped with a pharmaceutical active ingredient, preferably in particulate form, preferably in the form of lipid nanoparticles (LNP), obtainable by a method as defined above.

According to the present aspect of the present invention, the present invention also relates equally to the preferably lipid-based carrier system according to the invention, preferably in particulate form, preferably in the form of lipid nanoparticles (LNP), in particular for a pharmaceutically active ingredient, and/or to a preferably lipid-based carrier system loaded and/or equipped with a pharmaceutically active ingredient, preferably in particulate form, preferably in the form of lipid nanoparticles (LNP), in particular as defined above,
wherein the components of the carrier system are stored together, in particular aggregated, to form the carrier system, preferably in particulate form, preferably in the form of lipid nanoparticles (LNP), and/or wherein the carrier system is preferably in particulate form, preferably in the form of lipid nanoparticles (LNP),
wherein the components of the carrier system comprise the following substances:
(i) at least one phospholipid, preferably phosphatidylcholine;
   in particular wherein the phospholipid comprises an HLB value according to Griffin of at most 11, in particular at most 8, and/or in particular wherein the phospholipid comprises a Log P value of at least 0.55, in particular at least 0.80;
(ii) at least one non-ionic amphiphilic and/or surfactant substance, in particular selected surfactant PEGylated substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids and combinations and mixtures thereof, even more preferably selected from PEGylated fatty acids and salts and esters thereof;
   in particular wherein the non-ionic amphiphilic and/or surfactant substance comprises an HLB value according to Griffin of at least 13 and/or in particular wherein the non-ionic amphiphilic and/or surfactant substance comprises a Log P value of at most 0.46;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D):
   in particular wherein the steroid and/or secosteroid, in particular the sterol, comprises an HLB value according to Griffin of at most 1.5 and/or in particular wherein the steroid and/or secosteroid, in particular the sterol, comprises a Log P value of at least 5.40;
(iv) optionally at least one cationic lipid, in particular an amine-based cationic lipid, preferably a cationic lipid based on a tertiary amine;
   in particular wherein the cationic lipid comprises an HLB value according to Griffin of at most 4 and/or in particular wherein the cationic lipid comprises a Log P value of at least 1.76;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C₈- to C₁₆-mono-, C₈- to C₁₆-di- or C₈- to C₁₆-trifatty acid ester of glycerol, particularly preferred at least one C₈- to C₁₄-mono-, C₈- to C₁₄-di- or C₈- to C₁₄-trifatty acid ester of glycerol;
   in particular wherein the mono-, di- or tricarboxylic acid ester of glycerol comprises an HLB value according to Griffin of at most 8 and/or in particular wherein the mono-, di- or tricarboxylic acid ester of glycerol comprises a Log P value of at least 0.80;
in particular wherein the mono-, di- or tricarboxylic acid ester of glycerol comprises an HLB value according to Griffin of at most 8;
in particular wherein the carrier system or the carrier system loaded and/or equipped with the active pharmaceutical ingredient is contained and/or present in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant);
in particular wherein the carrier system forms with the liquid medium a preferably disperse composition, in particular a dispersion, which comprises the carrier system or the carrier system loaded and/or equipped with the pharmaceutically active agent and the liquid medium, and/or in particular wherein the carrier system forms with the liquid medium a dispersion of the carrier system or the carrier system loaded and/or equipped with a pharmaceutically active agent in the liquid medium.

In particular, with regard to the carrier system, it may be provided that the agglomeration, in particular aggregation, of the components to form the carrier system is effected with the input of energy, preferably input of mixing and/or stirring energy, in particular into the liquid medium comprising at least some of the components and/or substances of the carrier system and/or into a mixture comprising at least some of the components and/or substances of the carrier system, wherein the energy input, in particular mixing and/or stirring energy, has been set such that an at least substantially laminar flow is present, in particular in the liquid medium comprising at least some of the components and/or substances of the carrier system and/or in the mixture comprising at least some of the components and/or substances of the carrier system, that an at least substantially laminar flow has been present, in particular in the liquid medium comprising at least some of the components and/or substances of the carrier system and/or in the mixture comprising at least some of the components and/or substances of the carrier system, in particular with at least substantially complete avoidance of the formation of a non-laminar and/or turbulent flow and/or in particular with at least substantially complete avoidance of the formation of non-laminar and/or turbulent flow components.

In particular, with regard to the carrier system, it may be provided that a method and/or process control (production control), in particular continuous online and/or inline process control, is effected, in particular for monitoring the method status and/or method progress, preferably for monitoring and/or setting the formation of the particles of the carrier system, in particular lipid nanoparticles (LNP), and/or in particular for monitoring and/or setting process parameters, such as input of energy, preferably input of mixing and/or stirring energy, input and/or removal of heat energy, flow behavior, mass throughput, volume flows or the like; wherein the method and/or process control is effected by acquiring at least one process parameter, in particular at least two process parameters, wherein the process parameter is selected from the group of temperature, conductivity, viscosity, pH value, in particular in each case of the liquid medium, preferably together with the components and/or substances introduced therein, and/or the composition.

According to the invention, it can be provided in particular with regard to the carrier system,
in that the substance (i) comprises an HLB value according to Griffin in the range from 0.5 to 8, preferably in the range from 2 to 7, preferably in the range from 3 to 6, and/or in that the substance (i) comprises a Log P value in the range from 0.80 to 18.97, preferably in the range from 0.93 to 3.88, preferably in the range from 1.11 to 2.44; and/or
in that the substance (ii) comprises an HLB value according to Griffin in the range from 13 to 19, in particular in the range from 13 to 17.9, preferably in the range from 13.5 to 17.5, preferably in the range from 14 to 17, and/or in that the substance (ii) comprises a Log P value in the range from 0.30 to 0.46, in particular in the range from 0.32 to 0.46, preferably in the range from 0.33 to 0.44, preferably in the range from 0.34 to 0.42; and/or
that the substance (iii) comprises an HLB value according to Griffin in the range from 0.5 to 1.5, preferably in the range from 0.6 to 1.4, preferably in the range from 0.7 to 1.3, and/or that the substance (iii) comprises a Log P value in the range from 5.40 to 18.97, preferably in the range from 5.84 to 15.40, preferably in the range from 6.36 to 12.91; and/or
in that the substance (iv) comprises an HLB value according to Griffin in the range from 1.5 to 3.5, preferably in the range from 2 to 3, and/or in that the substance (iv) comprises a Log P value in the range from 2.05 to 5.40, preferably in the range from 2.44 to 3.88; and/or
in that the substance (v) comprises an HLB value according to Griffin in the range from 1 to 8, preferably in the range from 1.25 to 7, preferably in the range from 1.5 to 6.5, and/or in that the substance (v) comprises a Log P value in the range from 0.80 to 8.58, preferably in the range from 0.93 to 6.65, preferably in the range from 1.01 to 5.40.

In particular, the carrier system can be identified by at least one of the following features and/or measures (1a) to (5a), in particular by a combination of at least two, preferably at least three, preferably at least four, particularly preferred all five, of the following features and/or measures (1a) to (5a):
(1a) the substance (i) comprises an HLB value according to Griffin of at most 11, in particular at most 8, in particular in the range from 0.5 to 8, preferably in the range from 2 to 7, preferably in the range from 3 to 6, and/or the substance (i) comprises a Log P value of at least 0.55, in particular at least 0.80, in particular in the range from 0.80 to 18.97, preferably in the range from 0.93 to 3.88, preferably in the range from 1.11 to 2.44;
(2a) the substance (ii) comprises an HLB value according to Griffin of at least 13, in particular in the range from 13 to 19, preferably in the range from 13 to 17.9, preferably in the range from 13.5 to 17.5, particularly preferred in the range from 14 to 17, and/or the substance (ii) comprises a Log P value of at most 0.46, in particular in the range from 0.32 to 0.46, preferably in the range from 0.33 to 0.44, preferably in the range from 0.34 to 0.42;
(3a) the substance (iii) comprises an HLB value according to Griffin of at most 1.5, in particular in the range from 0.5 to 1.5, preferably in the range from 0.6 to 1.4, preferably in the range from 0.7 to 1.3, and/or the substance (iii) comprises a Log P value of at least 5.40, in particular in the range from 5.40 to 18.97, preferably in the range from 5.84 to 15.40, preferably in the range from 6.36 to 12.91;
(4a) the substance (iv) comprises an HLB value according to Griffin of at most 4, in particular in the range from 1 to 4, preferably in the range from 1.5 to 3.5, preferably in the range from 2 to 3, and/or the substance (iv) comprises a Log P value of at least 1.76, in particular in the range from 1.76 to 8.58, preferably in the range from 2.05 to 5.40, preferably in the range from 2.44 to 3.88;
(5a) the substance (v) comprises an HLB value according to Griffin of at most 8, in particular in the range from 1 to 8, preferably in the range from 1.25 to 7, preferably in the range from 1.5 to 6.5, and/or the substance (v) comprises a Log P value of at least 0.80, in particular in the range from 0.80 to 8.58, preferably in the range from 0.93 to 6.65, preferably in the range from 1.01 to 5.40.

According to the present aspect, the present invention also relates to the preferably lipid-based carrier system, preferably in particulate form, preferably in the form of lipid nanoparticles (LNP), in particular for a pharmaceutically active agent, and/or to a preferably lipid-based carrier system loaded or equipped with a pharmaceutically active agent, preferably in particulate form, preferably in the form of lipid nanoparticles (LNP), in particular as defined above,
wherein the components of the carrier system are stored together, in particular aggregated, to form the carrier system, preferably in particulate form, preferably in the form of lipid nanoparticles (LNP), and/or wherein the carrier system is preferably in particulate form, preferably in the form of lipid nanoparticles (LNP),
wherein the components of the carrier system comprise the following substances:
(i) at least one phospholipid, preferably phosphatidylcholine;
(ii) at least one non-ionic amphiphilic and/or surfactant substance, in particular selected from the group of polyglycerol esters, alkylpolyglycosides, amphiphilic and/or surfactant PEGylated (polyoxyethylated) substances and mixtures or combinations thereof; preferably selected from surfactant PEGylated substances, preferably selected from the group of PEGylated lipids, particularly preferred selected from the group of PEGylated fatty acids and their salts and esters, PEGylated triglycerides (PEGylated fats and PEGylated oils), PEGylated waxes, PEGylated phospholipids, PEGylated sphingolipids, PEGylated lipopolysaccharides and PEGylated isoprenoids as well as combinations and mixtures thereof, even more preferably selected from PEGylated fatty acids and salts and esters thereof, in particular polyol esters of PEGylated fatty acids;
(iii) optionally at least one steroid and/or secosteroid, in particular at least one sterol (sterol), preferably selected from the group of zoosterols, phytosterols and mycosterols and combinations and mixtures thereof, preferably cholesterol and/or phytosterol, and/or in particular at least one secosterol, preferably at least one calciferol (vitamin D);
(iv) optionally at least one cationic lipid, in particular an amine-based cationic lipid, preferably a cationic lipid based on a tertiary amine;
(v) optionally at least one mono-, di-, tri- or polycarboxylic acid ester of a polyol, in particular at least one mono-, di- or tricarboxylic acid ester of glycerol (mono-, di- or tricarboxylic acid glyceride), preferably at least one mono-, di- or trifatty acid ester of glycerol (mono-, di- or trifatty acid glyceride), preferably at least one C8- to C16-mono-, C8- to C16-di- or C8- to C16-trifatty acid ester of glycerol, particularly preferred at least one C8- to C14-mono-, C8- to C14-di- or C8- to C14-trifatty acid ester of glycerol;
wherein the carrier system is identified by at least one of the following features and/or measures (1a) to (5a), in particular by a combination of at least two, preferably at least three, preferably at least four, particularly preferred all five, of the following features and/or measures (1a) to (5a):
(1a) the substance (i) comprises an HLB value according to Griffin of at most 11, in particular at most 8, in particular in the range from 0.5 to 8, preferably in the range from 2 to 7, preferably in the range from 3 to 6, and/or the substance (i) comprises a Log P value of at least 0.55, in particular at least 0.80, in particular in the range from 0.80 to 18.97, preferably in the range from 0.93 to 3.88, preferably in the range from 1.11 to 2.44;
(2a) the substance (ii) comprises an HLB value according to Griffin of at least 13, in particular in the range from 13 to 19, preferably in the range from 13 to 17.9, preferably in the range from 13.5 to 17.5, particularly preferred in the range from 14 to 17, and/or the substance (ii) comprises a Log P value of at most 0.46, in particular in the range from 0.32 to 0.46, preferably in the range from 0.33 to 0.44, preferably in the range from 0.34 to 0.42;
(3a) the substance (iii) comprises an HLB value according to Griffin of at most 1.5, in particular in the range from 0.5 to 1.5, preferably in the range from 0.6 to 1.4, preferably in the range from 0.7 to 1.3, and/or the substance (iii) comprises a Log P value of at least 5.40, in particular in the range from 5.40 to 18.97, preferably in the range from 5.84 to 15.40, preferably in the range from 6.36 to 12.91;
(4a) the substance (iv) comprises an HLB value according to Griffin of at most 4, in particular in the range from 1 to 4, preferably in the range from 1.5 to 3.5, preferably in the range from 2 to 3, and/or the substance (iv) comprises a Log P value of at least 1.76, in particular in the range from 1.76 to 8.58, preferably in the range from 2.05 to 5.40, preferably in the range from 2.44 to 3.88;
(5a) the substance (v) comprises an HLB value according to Griffin of at most 8, in particular in the range from 1 to 8, preferably in the range from 1.25 to 7, preferably in the range from 1.5 to 6.5, and/or the substance (v) comprises a Log P value of at least 0.80, in particular in the range from 0.80 to 8.58, preferably in the range from 0.93 to 6.65, preferably in the range from 1.01 to 5.40;
in particular wherein the carrier system or the carrier system loaded and/or equipped with the active pharmaceutical ingredient is contained and/or present in a liquid medium, in particular in a medium which is liquid at a temperature in the range from 20° C. to 40° C., preferably room temperature (20° C.), and ambient pressure (1,013.25 hPa), preferably in the form of a dispersion medium (dispersant);
in particular wherein the carrier system forms with the liquid medium a preferably disperse composition, in particular a dispersion, which comprises the carrier system or the carrier system loaded and/or equipped with the pharmaceutically active agent and the liquid medium, and/or in particular wherein the carrier system forms with the liquid medium a dispersion of the carrier system or the carrier system loaded and/or equipped with a pharmaceutically active agent in the liquid medium.

According to the invention, the carrier system can behave in particular in such a way that the carrier system is characterized by a combination of the features and/or measures (1a) and (2a), in particular by a combination of the features and/or measures (1a), (2a) and (3a), preferably by a combination of the features and/or measures (1a), (2a), (3a) and (4a) or preferably by a combination of the features and/or measures (1a), (2a), (3a) and (5a), preferably by a combination of the features and/or measures (1a), (2a), (3a), (4a) and (5a).

With regard to the carrier system according to the invention, it may in particular be provided that the substances (i) and (iii) or the substances (i) and (iv) or the substances (i) and (v) have an average HLB value according to Griffin (respective arithmetic mean value of the HLB values according to Griffin of the aforementioned substance combinations) in the range from 1 to 10, in particular in the range from 2 to 9, preferably in the range from 3 to 8, and/or in that the substances (i) and (iii) or the substances (i) and (iv) or the substances (i) and (v) comprise a mean log P value (respective arithmetic mean of the log P values of the aforementioned substance combinations) in the range from 0.62 to 8.58, in particular in the range from 0.70 to 3.88, preferably in the range from 0.80 to 2.44; and/or
that the substances (i), (iii) and (iv) or the substances (i), (iii) and (v) or the substances (i), (iv) and (v) comprise an average HLB value according to Griffin (respective arithmetic mean value of the HLB values according to Griffin of the aforementioned substance combinations) in the range from 1 to 10, in particular in the range from 2 to 9, preferably in the range from 3 to 8, and/or that the substances (i), (iii) and (iv) or the substances (i), (iii) and (v) or the substances (i), (iv) and (v) comprise a mean log P value (respective arithmetic mean of the log P values of the aforementioned substance combinations) of the aforementioned substance combinations) in the range from 0.62 to 8.58, in particular in the range from 0.70 to 3.88, preferably in the range from 0.80 to 2.44; and/or
that the substances (i), (iii), (iv) and (v) comprise a mean HLB value according to Griffin (respective arithmetic mean value of the HLB values according to Griffin of the aforementioned substance combinations) in the range from 1 to 10, in particular in the range from 2 to 9, preferably in the range from 3 to 8, and/or that the substances (i), (iii), (iv) and (v) comprise a mean Log P value (respective arithmetic mean of the Log P values of the aforementioned substance combinations) of the aforementioned substance combinations) in the range from 0.62 to 8.58, in particular in the range from 0.70 to 3.88, preferably in the range from 0.80 to 2.44.

Similarly, it may be provided that the substance (i) comprises a critical packing parameter Pₖᵣ of at least 0.5, in particular at least 0.7, especially in the range from 0.7 to 1.225, preferably in the range from 0.77 to 1.12, preferably in the range from 0.84 to 1.05; and/or
that the substance (ii) comprises a critical packing parameter Pₖᵣ of at most 0.35, in particular in the range from 0.007 to 0.35, preferably in the range from 0.035 to 0.315, preferably in the range from 0.07 to 0.28; and/or
that the substance (iii) comprises a critical packing parameter Pₖᵣ of at least 1.155, in particular in the range from 1.155 to 1.225, preferably in the range from 1.162 to 1.218, preferably in the range from 1.169 to 1.211; and/or
that the substance (iv) comprises a critical packing parameter Pₖᵣ of at least 0.98, in particular in the range from 0.98 to 1.19, preferably in the range from 1.015 to 1.155, preferably in the range from 1.05 to 1.12; and/or
that the substance (v) comprises a critical packing parameter Pₖᵣ of at least 0.7, in particular in the range from 0.7 to 1.19, preferably in the range from 0.77 to 1.1725, preferably in the range from 0.805 to 1.155.

In particular, the carrier system can be identified by at least one of the following features and/or measures (1b) to (5b), in particular by a combination of at least two, preferably at least three, preferably at least four, particularly preferred all five, of the following features and/or measures (1b) to (5b):
(1b) the substance (i) comprises a critical packing parameter Pₖᵣ of at least 0.5, in particular at least 0.7, preferably in the range from 0.7 to 1.225, preferably in the range from 0.77 to 1.12, preferably in the range from 0.84 to 1.05;
(2b) the substance (ii) comprises a critical packing parameter Pₖᵣ of at most 0.35, in particular in the range from 0.007 to 0.35, preferably in the range from 0.035 to 0.315, preferably in the range from 0.07 to 0.28;
(3b) wherein the substance (iii) comprises a critical packing parameter Pₖᵣ of at least 1.155, in particular in the range from 1.155 to 1.225, preferably in the range from 1.162 to 1.218, preferably in the range from 1.169 to 1.211;
(4b) the substance (iv) comprises a critical packing parameter Pₖᵣ of at least 0.98, in particular in the range from 0.98 to 1.19, preferably in the range from 1.015 to 1.155, preferably in the range from 1.05 to 1.12;
(5b) the substance (v) comprises a critical packing parameter Pₖᵣ of at least 0.7, in particular in the range from 0.7 to 1.19, preferably in the range from 0.77 to 1.1725, preferably in the range from 0.805 to 1.155.

In particular, the carrier system can be realized by a combination of the features and/or measures (1b) and (2b), in particular by a combination of the features and/or measures (1b), (2b) and (3b), preferably by a combination of the features and/or measures (1b), (2b), (3b) and (4b) or preferably by a combination of the features and/or measures (1b), (2b), (3b) and (5b), preferably by a combination of the features and/or measures (1b), (2b), (3b), (4b) and (5b).

In particular, it may be provided that the carrier system comprises the substance (i) in an amount in the range from 0.5% to 50% by weight, in particular in the range from 1% to 40% by weight, preferably in the range from 1.25% to 30% by weight, preferably in the range from 1.5% to 25% by weight, based on the carrier system and/or based on the entirety of the components of the carrier system; and/or
in that the substance (i) is used in an amount such that the carrier system comprises the substance (i) in an amount in the range from 0.1 wt. % to 50 wt. %, in particular in the range from 0.25 wt. % to 40 wt. %, preferably in the range from 0.5 wt. % to 30 wt. %, preferably in the range from 1 wt. % to 25 wt. %, based on the carrier system and/or based on the entirety of the components of the carrier system.

For the carrier system, it can equally be in particular such that the carrier system comprises the substance (ii) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the entirety of the components of the carrier system; and/or in that the substance (ii) is used in an amount such that the carrier system comprises the substance (ii) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the entirety of the components of the carrier system.

In addition, it may be provided that the carrier system comprises the substance (iii) in an amount in the range from 0.2 wt. % to 40 wt. %, in particular in the range from 0.5 wt. % to 25 wt. %, preferably in the range from 1 wt. % to 20 wt. %, preferably in the range from 3 wt. % to 15 wt. %, based on the carrier system and/or based on the entirety of the components of the carrier system; and/or
in that the substance (iii) is used in an amount such that the carrier system comprises the substance (iii) in an amount in the range from 0.2 wt. % to 40 wt. %, in particular in the range from 0.5 wt. % to 25 wt. %, preferably in the range from 1 wt. % to 20 wt. %, preferably in the range from 3 wt. % to 15 wt. %, based on the carrier system and/or based on the entirety of the components of the carrier system.

Furthermore, it may be provided that the carrier system comprises the substance (iv) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the entirety of the components of the carrier system; and/or
in that the substance (iv) is used in an amount such that the carrier system comprises the substance (iv) in an amount in the range from 1 wt. % to 80 wt. %, in particular in the range from 2 wt. % to 75 wt. %, preferably in the range from 3 wt. % to 70 wt. %, preferably in the range from 5 wt. % to 65 wt. %, based on the carrier system and/or based on the entirety of the components of the carrier system.

In addition, it may be provided that the carrier system comprises the substance (v) in an amount in the range from 2 wt. % to 85 wt. %, in particular in the range from 4 wt. % to 80 wt. %, preferably in the range from 6 wt. % to 75 wt. %, preferably in the range from 8 wt. % to 70 wt. %, based on the carrier system and/or based on the entirety of the components of the carrier system; and/or
in that the substance (v) is used in an amount such that the carrier system comprises the substance (v) in an amount in the range from 2 wt. % to 85 wt. %, in particular in the range from 4 wt. % to 80 wt. %, preferably in the range from 6 wt. % to 75 wt. %, preferably in the range from 8 wt. % to 70 wt. %, based on the carrier system and/or based on the entirety of the components of the carrier system.

Further, it may also be provided that the weight-related quantitative ratio of substance (i) to substance (ii) [substance (i):substance (ii)] is in the range from 50:1 to 1:160, in particular in the range from 20:1 to 1:75, preferably in the range from 10:1 to 1:56, preferably in the range from 5:1 to 1:43, and/or is set to the aforementioned values; and/or
in that the substances (i) and (ii) are used in an amount such that the carrier system comprises the substances (i) and (ii) in a weight-related amount ratio of substance (i) to substance (ii) [substance (i):substance (ii)] in the range from 50:1 to 1:160, in particular in the range from 20:1 to 1:75, preferably in the range from 10:1 to 1:56, preferably in the range from 5:1 to 1:43.

In addition, it behaves in particular in such a way that the weight-related quantitative ratio of substance (i) to substance (iii) [substance (i):substance (iii)] is in the range from 250:1 to 1:80, in particular in the range from 80:1 to 1:25, preferably in the range from 30:1 to 1:16, preferably in the range from 8:1 to 1:10, and/or is set to the aforementioned values; and/or
in that the substances (i) and (iii) are used in an amount such that the carrier system comprises the substances (i) and (iii) in a weight-related amount ratio of substance (i) to substance (iii) [substance (i):substance (iii)] in the range from 250:1 to 1:80, in particular in the range from 80:1 to 1:25, preferably in the range from 30:1 to 1:16, preferably in the range from 8:1 to 1:10.

In particular, it may be provided that the weight-related quantitative ratio of substance (i) to substance (iv) [substance (i):substance (iv)] is in the range from 50:1 to 1:160, in particular in the range from 20:1 to 1:75, preferably in the range from 10:1 to 1:56, preferably in the range from 5:1 to 1:43, and/or is set to the aforementioned values; and/or
in that the substances (i) and (iv) are used in an amount such that the carrier system comprises the substances (i) and (iv) in a weight-related amount ratio of substance (i) to substance (iv) [substance (i):substance (iv)] in the range from 50:1 to 1:160, in particular in the range from 20:1 to 1:75, preferably in the range from 10:1 to 1:56, preferably in the range from 5:1 to 1:43.

In particular, it may be provided that the weight-related quantitative ratio of substance (i) to substance (v) [substance (i):substance (v)] is in the range from 25:1 to 1:170, in particular in the range from 10:1 to 1:80, preferably in the range from 5:1 to 1:60, preferably in the range from 4:1 to 1:46, and/or is set to the aforementioned values; and/or
in that the substances (i) and (v) are used in an amount such that the carrier system comprises the substances (i) and (v) in a weight-related amount ratio of substance (i) to substance (ii) [substance (i):substance (v)] in the range from 25:1 to 1:170, in particular in the range from 10:1 to 1:80, preferably in the range from 5:1 to 1:60, preferably in the range from 4:1 to 1:46.

According to the invention, it may also be provided that the weight-related quantitative ratio of substance (i) to substance (ii) to substance (iii) to substance (iv) [substance (i):substance (ii):substance (iii):substance (iv)] is in the range from (0.5 to 50):(1 to 80):(0.2 to 40):(1 to 80), in particular in the range from (1 to 40):(2 to 75):(0.5 to 25):(2 to 75), preferably in the range of (1.25 to 30):(3 to 70):(1 to 20):(3 to 70), preferably in the range of (1.5 to 25):(5 to 65):(3 to 15):(5 to 65), and/or is set to the aforementioned values; and/or
in that the substances (i), (ii), (iii) and (iv) are used in an amount such that the carrier system contains the substances (i), (ii), (iii) and (iv) in a weight-related quantity ratio of substance (i) to substance (ii) to substance (iii) to substance (iv) [substance (i):substance (ii):substance (iii):substance (iv)] in the range from (0.5 to 50):(1 to 80):(0.2 to 40):(1 to 80), in particular in the range from (1 to 40):(2 to 75):(0.5 to 25):(2 to 75), preferably in the range of (1.25 to 30):(3 to 70):(1 to 20):(3 to 70), preferably in the range of (1.5 to 25):(5 to 65):(3 to 15):(5 to 65).

In addition, it may also be provided in the context of the present invention that the weight-related quantitative ratio of substance (i) to substance (ii) to substance (iii) to substance (v) [substance (i):substance (ii):substance (iii):substance (v)] is in the range from (0.5 to 50):(1 to 80):(0.2 to 40):(2 to 85), in particular in the range from (1 to 40):(2 to 75):(0.5 to 25):(4 to 80), preferably in the range of (1.25 to 30):(3 to 70):(1 to 20):(6 to 75), preferably in the range of (1.5 to 25):(5 to 65):(3 to 15):(8 to 70), and/or is set to the aforementioned values; and/or
in that the substances (i), (ii), (iii) and (v) are used in an amount such that the carrier system contains the substances (i), (ii), (iii) and (v) in a weight-related quantity ratio of substance (i) to substance (ii) to substance (iii) to substance (v) [substance (i):substance (ii):substance (iii):substance (v)] in the range from (0.5 to 50):(1 to 80):(0.2 to 40):(2 to 85), in particular in the range from (1 to 40):(2 to 75):(0.5 to 25):(4 to 80), preferably in the range of (1.25 to 30):(3 to 70):(1 to 20):(6 to 75), preferably in the range of (1.5 to 25):(5 to 65):(3 to 15):(8 to 70).

In addition, however, it may also be provided that the weight-related quantitative ratio of substance (i) to substance (ii) to substance (iii) to substance (iv) to substance (v) [substance (i):substance (ii):Substance (iii):Substance (iv):substance (v)] in the range from (0.5 to 50):(1 to 80):(0.2 to 40):(1 to 80):(2 to 85), in particular (1 to 40):(2 to 75):(0.5 to 25):(2 to 75):(4 to 80), preferably (1.25 to 30):(3 to 70):(1 to 20):(3 to 70):(6 to 75), preferably (1.5 to 25):(5 to 65):(3 to 15):(5 to 65):(8 to 70), and/or is set to the aforementioned values; and/or
in that the substances (i), (ii), (iii) and (iv) are used in an amount such that the carrier system contains the substances (i), (ii), (iii) and (iv) in a weight-related quantity ratio of substance (i) to substance (ii) to substance (iii) to substance (iv) to substance (v) [substance (i):substance (ii):substance (iii):substance (iv)] in the range from (0.5 to 50):(1 to 80):(0.2 to 40):(1 to 80), in particular (1 to 40):(2 to 75):(0.5 to 25):(2 to 75), preferably (1.25 to 30):(3 to 70):(1 to 20):(3 to 70), preferably (1.5 to 25):(5 to 65):(3 to 15):(5 to 65).

In particular, the substance (ii) and the hydrophobic substances, in particular the hydrophobic amphiphilic substances, preferably selected from the group of substance (i), substance (iii), substance (iv), substance (v) and combinations thereof, may be employed in an amount such that the carrier system contains the substances in a weight-related quantitative ratio of substance (ii) to the hydrophobic substances, in particular hydrophobic amphiphilic substances, preferably selected from the group of substance (i), substance (iii), substance (iv), substance (v) and combinations thereof, preferably to substance (i) and/or substance (iii) and optionally substance (iv) and optionally substance (v), substance (iv) and optionally substance (v) [substance (ii): hydrophobic substances] in the range from 10:1 to 1:20, preferably in the range from 5:1 to 1:15, preferably in the range from 2:1 to 1:10.

In particular, substance (i) may be or comprise 1,2-distearoyl-sn-glycero-3-phosphocholine.

In addition, it may be provided according to the invention that the substance (ii) is and/or comprises a PEG stearate, in particular PEG 40 stearate, and/or a macrogol stearate, in particular macrogol 40 stearate; and/or wherein the substance (ii) is and/or comprises PEG 2000-DMG (1,2-dimyristoyl-rac-glycero-3-methoxypolyethyleneglycol-2000); and/or wherein the substance (ii) is and/or comprises a cetromacrogol (polyethylene glycol hexadecyl ether); and/or wherein the substance (ii) is and/or comprises 2-[(polyethylene glycol)-2000]-N,N-ditetradecylacetamide (ALC-0159); and/or wherein the substance (ii) is and/or comprises a polaxamer; and/or wherein the substance (ii) is or comprises an alkylpolyglycoside, in particular C₈- to C₁₄-alkylpolyglycoside, preferably C₁₀- to C₁₂-alkylpolyglycoside; and/or wherein the substance (ii) is or comprises an ethoxylated sorbitan fatty acid ester, in particular polysorbate, preferably polysorbate 80 and/or polysorbate 20; and/or
that the substance (iii) is or comprises cholesterol or phytosterol or mixtures thereof, in particular phytosterol; and/or
wherein the substance (iv) is or comprises [(4-hydroxybutyl)azandiyl]-bis(hexane-6,1-diyl)-bis(2hexyldecanoate-) (ALC-0315); and/or wherein the substance (iv) is or comprises-heptadecan-9-yl-8-((2-hydroxyethyl)(6-oxo-6-(undecyloxy)-hexyl)amino)octanoate (SM-102); and/or
wherein the substance (v) is or comprises glycerol monolaurate; and/or wherein the substance (v) is or comprises capric acid/caprylic acid triglyceride.

In particular, the substance (iv) or the cationic lipid may comprise at least one tertiary aminofunctional group which can be quaternized in particular.

According to the invention, the active ingredient, in particular as substance (vi), can be a component of the carrier system and/or form a component of the carrier system and/or wherein the carrier system comprises the active ingredient, in particular as substance (vi).

In particular, it may be provided according to the invention that the active ingredient is a therapeutic and/or prophylactic active ingredient, in particular a therapeutic and/or prophylactic pharmaceutical active ingredient;
in particular wherein the active ingredient is an at least substantially water-insoluble, preferably an at least substantially water-insoluble amphiphilic active ingredient; and/or in particular wherein the active ingredient is a water-soluble active ingredient; and/or
in particular wherein the active ingredient is selected from the group of vaccine active ingredients, antibiotic active ingredients, antifungal active ingredients, antiviral active ingredients, antiphlogistic active ingredients, antipyretic active ingredients, antiallergic active ingredients, anticancer active ingredients, hormone active ingredients, anticoagulant active ingredients, vitamins, minerals, UV-absorbing substances and mixtures thereof and combinations thereof, preferably vaccine active ingredients; and/or
in particular wherein the active ingredient is selected from the group of RNA, in particular messenger RNA (mRNA); retinol; tocopherol; corticosteroids, in particular cortisone; antibiotics, in particular tetracyclines and benzoylpenicillins; diclofenac; estradiol, estradiol hemihydrate, salicylic acid, acetylsalicylic acid; indomethacin; essential oils; in particular amphiphilic UVA filters and UVB filters; nicotinamide; and mixtures and combinations thereof; and/or
in particular wherein the active ingredient is a vaccine active ingredient, in particular a nucleic acid-based vaccine active ingredient and/or gene-based vaccine active ingredient, preferably a vaccine active ingredient encoding an antigen of a pathogen and/or a pathogen, in particular an antigen of a virus; and/or
in particular wherein the vaccine active ingredient is selected from the group consisting of DNA; RNA, preferably messenger RNA (mRNA); viral vectors; plasmids, preferably plasmid DNA (pDNA); preferably RNA; preferably mRNA; and mixtures and combinations thereof; and/or
in particular wherein the active ingredient is an mRNA-based vaccine active ingredient and/or mRNA, in particular wherein the mRNA is single-stranded and/or in particular wherein the mRNA comprises a 5′-cap structure; and/or in particular wherein the active ingredient is an mRNA-based vaccine active ingredient and/or mRNA, in particular wherein the mRNA encodes a protein, in particular antigen, of a coronavirus, preferably SARS-CoV-2, in particular wherein the protein, in particular antigen, encodes the spike protein, in particular membrane-anchored spike protein (spike glycoprotein or S protein or S glycoprotein) of the coronavirus, preferably SARS-CoV-2, at least partially, preferably at least substantially completely; and/or
in particular wherein the active ingredient is a protein and/or antigen, in particular of a pathogenic agent, such as a bacterium, virus or the like.

According to the invention, the carrier system may comprise the active ingredient, in particular as substance (vi), and/or the substance (vi) in an amount in the range from 0.01 wt. % to 80 wt. %, in particular in the range from 0.1 wt. % to 70 wt. %, preferably in the range from 0.5 wt. % to 60 wt. %, preferably in the range from 1 wt. % to 50 wt. %, based on the carrier system and/or based on the entirety of the components of the carrier system.

According to the invention, in particular mRNA can be used as the active ingredient. The excellent properties of the carrier system also enable high cell uptake and bioavailability.

In particular, the active ingredient can be a vitamin A or vitamin A derivative, especially retinol or vitamin A1. This also enables high absorption of the active ingredient.

In addition, it may be provided according to the invention that the carrier system-comprises as a further component, in particular as substance (vii), at least one fatty alcohol, in particular C₈- to C₂₅-fatty alcohol, preferably at least one branched fatty alcohol, preferably octyldodecanol, particularly preferred 2octyldocen-1-ol-, in particular in an amount in the range from 1 wt.-% to 80 wt.-%, preferably in the range from 5 wt.-% to 70 wt.-%, preferably in the range from 10 wt.-% to 60 wt.-%, based on the carrier system and/or in an amount in the range from 1 wt.-% to 80 wt.-%, preferably in the range from 5 wt.-% to 70 wt.-%, preferably in the range from 10 wt.-% to 60 wt.-%.-% to 80% by weight, preferably in the range from 5% to 70% by weight, preferably in the range from 10% to 60% by weight, based on the carrier system and/or based on the total of the components of the carrier system;
in particular wherein the fatty alcohol, preferably octyldodecanol, comprises an HLB value according to Griffin in the range from 1 to 5, in particular in the range from 2 to 4, and/or in particular wherein the fatty alcohol, preferably octyldodecanol, comprises a Log P value in the range from 1.36 to 8.58, in particular in the range from 1.76 to 3.88; and/or
in particular wherein the fatty alcohol, preferably octyldodecanol, comprises a critical packing parameter Pₖᵣ in the range from 0.91 to 1.19, in particular in the range from 0.98 to 1.12.

In general, the carrier system can comprise as a further component, in particular as substance (viii), at least one surface-active amphiphilic substance, in particular surface-active and non-micelle-forming amphiphilic substance, preferably at least one sorbitan fatty acid ester, preferably at least one sorbitan fatty acid ester with or based on saturated and/or unsaturated C₈- to C₁₈-fatty acids, in particular C₁₀- to C₁₄-fatty acids, particularly preferred sorbitan caprylate, in particular in an amount in the range of 0.1 wt.-% to 30 wt.-%, preferably at least one sorbitan fatty acid ester, preferably at least one sorbitan fatty acid ester with or based on saturated and/or unsaturated C- to C-fatty acids, in particular C- to C-fatty acids, particularly preferred sorbitan caprylate, in particular in an amount in the range of 0.1 wt.-% to 30 wt. %, preferably in the range from 1 wt. % to 20 wt. %, preferably in the range from 5 wt.-% to 15 wt. %, based on the carrier system and/or based on the total of the components of the carrier system;
in particular wherein the surface-active amphiphilic substance, preferably the sorbitan fatty acid ester, comprises an HLB value according to Griffin in the range from 2 to 5, in particular in the range from 3 to 4, and/or in particular wherein the surface-active amphiphilic substance, preferably the sorbitan fatty acid ester, comprises a Log P value in the range from 1.36 to 3.88, in particular in the range from 1.76 to 2.44; and/or
in particular wherein the surface-active amphiphilic substance, preferably the sorbitan fatty acid ester, comprises a critical packing parameter Pₖᵣ in the range from 0.91 to 1.12, in particular in the range from 0.98 to 1.05.

In addition, it can behave in such a way that the carrier system comprises as a further component, in particular as substance (ix), at least one ether, in particular ether of a diol or polyol, preferably ether of a bicyclic diol, preferably dialkyl isosorbide, particularly preferably dimethyl isosorbide (DMI), particularly more preferably 2,5-dimethyl isosorbide, in particular in an amount in the range from 2.5 wt.-% to 30 wt.-%, preferably in the range from 5 wt.-% to 25 wt.-%, preferably in the range from 10 wt.-% to 20 wt.-%, based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system and/or based on the carrier system. % to 30 wt. %, preferably in the range from 5 wt. % to 25 wt. %, preferably in the range from 10 wt. % to 20 wt. %, based on the carrier system and/or based on all the components of the carrier system.

According to the invention, it may be provided that the carrier system comprises as a further component, in particular as substance (x), at least one alcohol, in particular wherein the alcohol is selected from the group of monohydric alcohols, polyhydric alcohols, in particular polyols, and mixtures thereof and combinations thereof; and/or wherein the carrier system comprises as a further component, in particular as substance (x), at least one polyol;
wherein the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof; and/or
wherein the alcohol, in particular the polyol, is selected from the group consisting of propylene glycol, polyethylene glycol, sorbitol, mannitol, glycerol, sucrose, panthenol and mixtures thereof and combinations thereof; and/or
in particular wherein the alcohol, in particular the polyol, is glycerol; and/or
in particular wherein the alcohol, in particular the polyol, is panthenol; and/or
in particular wherein the alcohol, in particular the polyol, is present and/or used in an amount in the range from 1 wt. % to 90 wt. %, in particular in the range from 2 wt. % to 70 wt. %, preferably in the range from 3 wt. % to 60 wt. %, preferably in the range from 5 wt. % to 55 wt. %, relative to the carrier system, in particular relative to the entirety of the components of the carrier system; and/or
in particular wherein the alcohol, in particular the polyol, is used in an amount such that the weight-related quantitative ratio of alcohol, in particular the polyol, to carrier system, in particular the entirety of the components of the carrier system, [alcohol, in particular polyol:carrier system, in particular the entirety of the components of the carrier system] is in the range from 1:150 to 10:1, in particular in the range from 1:100 to 5:1, preferably in the range from 1:50 to 1:1, preferably in the range from 1:20 to 1:5, and/or is set.

With regard to the carrier system, the liquid medium can be designed and/or present as a continuous phase.

In this context, the liquid medium may be present or used in an amount 0.05 times to 10 times, in particular 0.1 times to 5 times, especially 0.15 times to 4 times, preferably 0.2 times to 3 times, the amount by weight of the carrier system, in particular the amount by weight of the entirety of the components of the carrier system.

In addition, the liquid medium can be used in an amount such that the weight-related amount ratio of carrier system, in particular the entirety of the components of the carrier system, to liquid medium [carrier system, in particular the entirety of the components of the carrier system:liquid medium] is in the range from 1:20 to 50:1, in particular in the range from 1:10 to 20:1, preferably in the range from 1:5 to 10:1, preferably in the range from 1:4 to 5:1, and/or is set.

According to the invention, it may in particular be provided that the liquid medium comprises water and/or at least one alcohol, preferably water and at least one alcohol;
wherein the alcohol is selected from the group consisting of monohydric alcohols, polyhydric alcohols, in particular polyols, and mixtures and combinations thereof; and/or in particular wherein the alcohol is a polyhydric alcohol, in particular polyol.

According to the invention, it may in particular be provided that the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose;

panthenol; and mixtures and combinations thereof; and/or
that the alcohol, in particular the polyol, is selected from the group of propylene glycol, polyethylene glycol, sorbitol, mannitol, glycerol, sucrose, panthenol and mixtures and combinations thereof; and/or
that the alcohol, in particular the polyol, is glycerol; and/or
that the alcohol, in particular the polyol, is panthenol and/or
wherein the alcohol is not a C₁- to C₄-monoalcohol, preferably not an ethanol, and/or in particular wherein the alcohol does not comprise a C₁- to C₄-monoalcohol, in particular not an ethanol; and/or
that the liquid medium does not comprise any C₁- to C₄-monoalcohol, in particular no ethanol.

In addition, it may be provided according to the invention that the liquid medium contains the water in an amount in the range from 10 wt. % to 99.5 wt. %, in particular in the range from 20 wt. % to 99 wt. %, preferably in the range from 30 wt.-% to 97 wt. %, preferably in the range from 35 wt. % to 95 wt. %, based on the liquid medium; and/or
that the liquid medium contains the alcohol, in particular the polyol, in an amount in the range from 0.5 wt. % to 90 wt. %, in particular in the range from 1 wt. % to 80 wt. %, preferably in the range from 3 wt. % to 70 wt. %, preferably in the range from 5 wt. % to 65 wt. %, based on the liquid medium; and/or
in that the liquid medium comprises or consists of a mixture of water and at least one alcohol, in particular polyol, in particular wherein the weight-related quantitative ratio of water to alcohol, in particular polyol, [water:alcohol, in particular polyol] is in the range from 200:1 to 1:50, in particular in the range from 100:1 to 1:20, preferably in the range from 50:1 to 1:5, preferably in the range from 20:1 to 1:1, in particular with respect to the liquid medium, and/or is set.

In addition, with respect to the carrier system according to the invention, it may also be such that the liquid medium comprises at least one ether, in particular ether of a diol or polyol, preferably ether of a bicyclic diol, preferably dialkyl isosorbide, particularly preferably dimethyl isosorbide, even more preferably 2,5-dimethyl isosorbide, in particular in an amount in the range from 0.5 wt.-% to 25 wt.-%, preferably in the range from 1 wt.-% to 10 wt.-%, based on the liquid medium. % to 25 wt. %, preferably in the range from 1 wt. % to 10 wt. %, preferably in the range from 1 wt. % to 10 wt. %, based on the liquid medium.

In particular, the liquid medium may comprise at least one antioxidant, in particular ascorbic acid (vitamin C), in particular in an amount in the range from 0.1 wt. % to 10 wt. %, preferably in the range from 0.2 wt. % to 5 wt. %, preferably in the range from 0.5 wt. % to 2 wt. %, based on the liquid medium.

Furthermore, the liquid medium may comprise at least one biopolymer, in particular selected from the group of alginates, hyaluronic acid and chitosan (as well as their respective salts and derivatives) and mixtures and combinations thereof, in particular in an amount in the range from 0.1 wt. % to 50 wt. %, in particular in the range from 0.5 wt. % to 30 wt. %, preferably in the range from 1 wt. % to 10 wt. %, based on the liquid medium.

According to the invention, it may in particular be provided that the carrier system and/or the liquid medium and/or the composition, in particular as defined above, is/are at least substantially free of aliphatic monoalcohols, in particular at least substantially free of aliphatic C₁- to C₄-monoalcohols, preferably at least substantially free of ethanol.

In addition, it may be provided that the carrier system or the liquid medium or the composition, in particular as defined above, is or are at least substantially free of halogenated hydrocarbons, in particular at least substantially free of chlorinated hydrocarbons, preferably at least substantially free of chloroform.

In general, the carrier system also behaves in such a way that the agglomeration, in particular aggregation, of the components is such that the carrier system results in particulate form, in particular in the form of lipid nanoparticles (LNP), preferably lipid nanoparticles (LNP) in the form of micelles, vesicles and/or disk-shaped lipid nanoparticles (LNP) (disk lipid nanoparticles or disk LNP); and/or disk-LNP), preferably disk-shaped lipid nanoparticles (LNP); and/or
that the carrier system is present in particulate form, in particular in the form of lipid nanoparticles (LNP), preferably lipid nanoparticles (LNP) in the form of micelles, vesicles and/or disk-shaped lipid nanoparticles (LNP) (disk lipid nanoparticles or disk LNP), preferably disk-shaped lipid nanoparticles (LNP).

In general, the particle sizes (absolute), in particular the hydrodynamic diameters (absolute), of the particles of the carrier system, preferably lipid nanoparticles (LNP), can comprise at least essentially a normal distribution and/or Gaussian distribution, in particular wherein the particle sizes, in particular the hydrodynamic diameters, are determined using light diffraction (light diffractometry), in particular laser diffraction, preferably dynamic light scattering (DLS), preferably according to ISO 22412:2017 and/or DIN ISO 22412:2018-09, can be determined.

According to the invention, it is also particularly preferred that the particles of the carrier system, preferably the lipid nanoparticles (LNP), have a particle size (absolute), in particular a hydrodynamic diameter (absolute), in the range from 5 nm to 10,000 nm, in particular in the range from 10 nm to 5,000 nm, preferably in the range from 20 nm to 3,000 nm, preferably in the range from 50 nm to 2,000 nm, particularly preferably in the range from 75 nm to 1.500 nm, particularly more preferably in the range from 90 nm to 1,250 nm, further preferably in the range from 100 nm to 1,000 nm, again further preferably in the range from 100 nm to 900 nm, determined in particular using light diffraction (light diffractometry), in particular laser diffraction, preferably dynamic light scattering (DLS), preferably in accordance with ISO 22412:2017 and/or DIN ISO 22412:2018-09.

In addition, it can behave in such a way that the particles of the carrier system, preferably the lipid nanoparticles (LNP), have an average particle size, in particular an average hydrodynamic diameter, in the range from 5 nm to 2,900 nm, in particular in the range from 10 nm to 2,500 nm, preferably in the range from 20 nm to 2.000 nm, preferably in the range from 50 nm to 1,500 nm, particularly preferred in the range from 50 nm to 1,000 nm, in particular determined using light diffraction (light diffractometry), in particular laser diffraction, preferably dynamic light scattering (DLS), preferably in accordance with ISO 22412:2017 and/or DIN ISO 22412:2018-09.

In addition, it may be provided that the variance of the particle size, in particular the variance of the hydrodynamic diameter, of the particles of the carrier system, preferably the lipid nanoparticles (LNP), is at most 45%, in particular at most 35%, preferably at most 25%, preferably at most 20%, particularly preferably at most 15%, particularly more preferably at most 10%, in particular in relation to the mean particle size or the mean hydrodynamic diameter, and/or in particular determined using light diffraction (light diffractometry, in particular laser diffraction, preferably dynamic light scattering (DLS), preferably in accordance with the mean hydrodynamic diameter and/or in particular determined using light diffraction (light diffractometry), in particular laser diffraction, preferably dynamic light scattering (DLS), preferably in accordance with ISO 22412:2017 and/or DIN ISO 22412:2018-09; and/or
that the variance of the particle size, in particular the variance of the hydrodynamic diameter, of the particles of the carrier system, preferably of the lipid nanoparticles (LNP), is in the range from 5% to 45%, in particular in the range from 10% to 40%, preferably in the range from 15% to 35%, preferably in the range from 20% to 35%, in particular with respect to the mean particle size or the mean hydrodynamic diameter and/or in particular determined using light diffraction (light diffractometry), in particular laser diffraction, preferably dynamic light scattering (DLS), preferably in accordance with ISO 22412:2017 and/or DIN ISO 22412:2018-09.

Furthermore, it may also be provided that the particles of the carrier system, preferably the lipid nanoparticles (LNP), have an average particle size D50, in particular an average hydrodynamic diameter D50, in the range from 2.5 nm to 950 nm, in particular in the range from 7.5 nm to 850 nm, preferably in the range from 10 nm to 800 nm, preferably in the range from 15 nm to 750 nm, in particular determined using light diffraction (light diffractometry), in particular laser diffraction, preferably dynamic light scattering (DLS), preferably in accordance with ISO 22412:2017 and/or DIN ISO 22412:2018-09; and/or that the particles of the carrier system, preferably the lipid nanoparticles (LNP), have an average particle size D90, in particular an average hydrodynamic diameter D90, in the range from 1 nm to 1.000 nm, in particular in the range from 5 nm to 900 nm, preferably in the range from 7.5 nm to 850 nm, preferably in the range from 10 nm to 800 nm, in particular determined using light diffraction (light diffractometry), in particular laser diffraction, preferably dynamic light scattering (DLS), preferably in accordance with ISO 22412:2017 and/or DIN ISO 22412:2018-09.

According to the invention, it can also behave in such a way that the agglomeration, in particular aggregation, of the components is such that a lyotropic liquid-crystalline structure in particular results on the basis of the carrier system and the liquid medium; and/or
that the carrier system together with the liquid medium is present as a lyotropic liquid-crystalline structure based on the carrier system and the liquid medium and/or comprises this structure.

In particular, it may be provided according to the invention that the carrier system is contained and/or supplied in a liquid medium, in particular as defined above, in particular that the carrier system forms a composition, in particular a pharmaceutical composition, with the liquid medium; and/or
in particular that the composition is a preferably disperse composition, in particular a dispersion, preferably as defined above, which comprises the carrier system or the carrier system loaded and/or equipped with the pharmaceutically active substance and the liquid medium, and/or wherein the composition, in particular as defined above, is in the form of a dispersion of the carrier system or the carrier system loaded and/or equipped with a pharmaceutically active substance in the liquid medium.

In particular, it may be provided according to the invention that the composition comprises the carrier system, preferably the particles, preferably the lipid nanoparticles, in an amount in the range from 5 wt. % to 95 wt. %, in particular in the range from 10 wt. % to 90 wt. %, preferably in the range from 15 wt. % to 85 wt. %, preferably in the range from 20 wt. % to 80 wt. %, particularly preferred in the range from 25 wt. % to 75 wt. %, based on the composition; and/or
that the carrier system is present in the composition in particulate form, in particular in the form of lipid nanoparticles (LNP), preferably lipid nanoparticles (LNP) in the form of micelles, vesicles and/or discoidal (disk-shaped) lipid nanoparticles (LNP) (disk lipid nanoparticles or disk LNP), preferably discoidal (disk-shaped) lipid nanoparticles (LNP), in particular as defined above.

With respect to the composition, it may also be such that the composition comprises a water activity of less than 1, in particular at most 0.9, preferably at most 0.7, preferably at most 0.5, particularly preferred at most 0.3; and/or
that the composition comprises a water activity in the range from 0 to 1, in particular in the range from 0 to 0.9, preferably in the range from 0 to 0.7, preferably in the range from 0 to 0.5, particularly preferred in the range from 0 to 0.3;
in particular determined in accordance with ISO 21807:2004 and/or ISO 18787:2017 and/or 21 CFR Part 11, in particular as defined in ISO 29621:2017.

In particular, the carrier system and/or the composition, in particular the carrier system, may comprise a Log P value, in particular total Log P value, in the range from 0.1 to 5, in particular in the range from 0.1 to 5, in particular in the range from 0.2 to 4, preferably in the range from 0.3 to 3, particularly preferred in the range from 0.4 to 2. In particular, it may also be provided that the components and/or substances of the carrier system, in particular substances (i), (ii), (iii), (iv) and/or (v), are selected with the proviso that the resulting carrier system comprises a Log P value, in particular a total Log P value, of at most 5, in particular at most 4, preferably at most 3, particularly preferred at most 2, further preferred at most 1. Furthermore, it may be provided that the components and/or substances of the carrier system, in particular the substances (i), (ii), (iii), (iv) and/or (v), are selected with the proviso that the resulting carrier system comprises a Log P value, in particular a total Log P value, in the range from 0.1 to 5, in particular in the range from 0.2 to 4, preferably in the range from 0.3 to 3, particularly preferred in the range from 0.4 to 2.

Furthermore, according to a further aspect of the present invention, the subject-matter of the present invention is also the composition according to the invention, in particular a pharmaceutical composition, preferably a drug or medicament, wherein the composition comprises at least one preferably lipid-based carrier system, preferably in particulate form, preferably in the form of lipid nanoparticles (LNP), in particular for a pharmaceutically active substance; and/or at least one preferably lipid-based carrier system loaded and/or equipped with a pharmaceutical active ingredient, preferably in particulate form, preferably in the form of lipid nanoparticles (LNP), according to one of the claims 82 to 129 in a liquid medium, in particular in a medium at a temperature in the range from 20° C. to 40° C., preferably room temperature (20° C.), and ambient pressure (1.013.25 hPa), preferably in the form of a dispersion medium (dispersant).

In particular, the composition is preferably a disperse composition, in particular a dispersion comprising the carrier system or the carrier system loaded and/or equipped with the pharmaceutically active agent and the liquid medium. In particular, the composition according to the invention is in the form of a dispersion of the carrier system or the carrier system loaded and/or equipped with a pharmaceutically active agent in the liquid medium.

The composition may comprise the carrier system, preferably the particles, preferably the lipid nanoparticles, in an amount in the range from 5 wt. % to 95 wt. %, more preferably in the range from 10 wt. % to 90 wt. %, preferably in the range from 15 wt. % to 85 wt. %, more preferably in the range from 20 wt. % to 80 wt. %, particularly preferred in the range from 25 wt. % to 75 wt. %, based on the composition.

In addition, the carrier system may be present in the composition in particulate form, in particular in the form of lipid nanoparticles (LNP), preferably lipid nanoparticles (LNP) in the form of micelles, vesicles and/or discoidal (disk-shaped) lipid nanoparticles (LNP) (disk lipid nanoparticles or disk LNP), preferably discoidal (disk-shaped) lipid nanoparticles (LNP), in particular as defined above.

In particular, the composition may comprise a water activity of less than 1, in particular at most 0.9, preferably at most 0.7, preferably at most 0.5, particularly preferred at most 0.3. In particular, the composition may comprise a water activity in the range from 0 to 1, especially in the range from 0 to 0.9, preferably in the range from 0 to 0.7, preferably in the range from 0 to 0.5, particularly preferred in the range from 0 to 0.3. The water activity can be determined in particular according to ISO 21807:2004 and/or according to ISO 18787:2017 and/or 21 CFR Part 11, in particular as defined in ISO 29621:2017.

In general, the active ingredient can be a component of the carrier system or form a component of the carrier system. In particular, the carrier system may comprise or contain the active ingredient.

According to the invention, with regard to the composition according to the invention, the active ingredient may be a therapeutic and/or prophylactic active ingredient, in particular a therapeutic and/or prophylactic pharmaceutical active ingredient,
in particular wherein the active ingredient is an at least substantially water-insoluble, preferably an at least substantially water-insoluble amphiphilic active ingredient; and/or in particular wherein the active ingredient is a water-soluble active ingredient; and/or
wherein the active ingredient is selected from the group consisting of vaccine active ingredients (vaccine active ingredients), antibiotic active ingredients, antifungal active ingredients, antiviral active ingredients, antiphlogistic active ingredients, antipyretic active ingredients, antiallergic active ingredients anticancer agents, hormonal agents, anticoagulant agents, antitumor agents (such as cytostatics), food supplements, vitamins, minerals, UV-absorbing substances and mixtures thereof and combinations thereof, preferably vaccine agents (vaccine agents); and/or
in particular wherein the active ingredient is selected from the group of RNA, in particular messenger RNA (mRNA); retinol; tocopherol; corticosteroids, in particular cortisone; antibiotics, in particular tetracyclines and benzoylpenicillins; diclofenac; estradiol, estradiol hemihydrate, salicylic acid, acetylsalicylic acid; indomethacin; essential oils; in particular amphiphilic UVA filters and UVB filters; nicotinamide; and mixtures and combinations thereof; and/or
in particular wherein the active substance is a vaccine active substance (vaccine active substance), in particular a nucleic acid-based vaccine active substance and/or gene-based vaccine active substance, preferably a vaccine active substance encoding an antigen of a pathogen and/or a pathogenic agent, in particular an antigen of a virus; and/or
in particular wherein the vaccine active ingredient is selected from the group consisting of DNA; RNA, preferably messenger RNA (mRNA); viral vectors; plasmids, preferably plasmid DNA (pDNA); preferably RNA; preferably mRNA; and mixtures and combinations thereof; and/or
in particular wherein the active ingredient is an mRNA-based vaccine active ingredient and/or mRNA, in particular wherein the mRNA is single-stranded and/or in particular wherein the mRNA comprises a 5′-cap structure; and/or in particular wherein the active ingredient is an mRNA-based vaccine active ingredient and/or mRNA, in particular wherein the mRNA encodes a protein, in particular antigen, of a coronavirus, preferably SARS-CoV-2, in particular wherein the protein, in particular antigen, encodes the spike protein, in particular membrane-anchored spike protein (spike glycoprotein or S protein or S glycoprotein) of the coronavirus, preferably SARS-CoV-2, at least partially, preferably at least substantially completely; and/or
in particular wherein the active ingredient is a protein and/or antigen, in particular of a pathogenic agent, such as a bacterium, virus or the like.

In this context, the carrier system may comprise the active ingredient in an amount in the range from 0.01 wt. % to 80 wt. %, in particular in the range from 0.1 wt. % to 70 wt. %, preferably in the range from 0.5 wt. % to 60 wt. %, preferably in the range from 1 wt. % to 50 wt. %, based on the carrier system and/or based on the entirety of the components of the carrier system.

In addition, the composition may comprise the active ingredient in an amount in the range from 0.001 wt. % to 75 wt. %, in particular in the range from 0.01 wt. % to 65 wt. %, preferably in the range from 0.1 wt. % to 55 wt. %, preferably in the range from 0.5 wt. % to 50 wt. %, based on the composition.

Furthermore, the liquid medium can be designed or present as a continuous phase.

According to the invention, it may in particular be provided that the liquid medium is present or used in an amount by weight which is 0.05 times to 10 times, in particular 0.1 times to 5 times, especially 0.15 times to 4 times, preferably 0.2 times to 3 times, the amount by weight of the carrier system, in particular the amount by weight of the entirety of the components of the carrier system.

In this respect, the liquid medium can be used in an amount such that the weight-related amount ratio of carrier system, in particular the entirety of the components of the carrier system, to liquid medium [carrier system, in particular the entirety of the components of the carrier system:liquid medium] is in the range from 1:20 to 50:1, in particular in the range from 1:10 to 20:1, preferably in the range from 1:5 to 10:1, preferably in the range from 1:4 to 5:1, and/or is set.

With regard to the composition according to the invention, the liquid medium may comprise water or at least one alcohol, preferably water and at least one alcohol.

In this context, the alcohol may be selected from the group of monohydric alcohols, polyhydric alcohols, in particular polyols, and mixtures and combinations thereof.

In a preferred manner according to the invention, the alcohol is a polyhydric alcohol, in particular a polyol.

In this respect, it can behave according to the invention in particular in such a way that the alcohol, in particular the polyol, is selected from the group of alkylene glycols, in particular propylene glycol; polyalkylene glycols, in particular polyethylene glycol; sugar alcohols, in particular sorbitol, mannitol and glycerol; saccharides, in particular disaccharides, preferably sucrose; panthenol; and mixtures and combinations thereof.

In a preferred manner according to the invention, the alcohol, in particular the polyol, is selected from the group of propylene glycol, polyethylene glycol, sorbitol, mannitol, glycerol, sucrose, panthenol and mixtures and combinations thereof.

According to the invention, it may be provided that alcohol, in particular the polyol, is glycerol.

According to the invention, it may in particular be provided that the alcohol, in particular the polyol, is panthenol. Reference can also be made to the advantages and properties of panthenol described above.

With regard to the liquid medium of the composition, it also applies in particular that the alcohol is not a C₁- to C₄-monoalcohol, preferably not ethanol, or that the alcohol does not comprise a C₁- to C₄-monoalcohol, in particular not ethanol.

In particular, it is provided according to the invention that the liquid medium does not comprise any C1- to C4-monoalcohol, in particular no ethanol.

In general, the liquid medium may contain the water in an amount in the range from 10 wt. % to 99.5 wt. %, in particular in the range from 20 wt. % to 99 wt. %, preferably in the range from 30 wt. % to 97 wt. %, preferably in the range from 35 wt. % to 95 wt. %, based on the liquid medium.

In addition, the liquid medium may contain the alcohol, in particular the polyol, in an amount in the range from 0.5 wt. % to 90 wt. %, in particular in the range from 1 wt. % to 80 wt. %, preferably in the range from 3 wt. % to 70 wt. %, preferably in the range from 5 wt. % to 65 wt. %, based on the liquid medium.

Furthermore, the liquid medium may comprise or consist of a mixture of water and at least one alcohol, in particular polyol, in particular wherein the weight-related quantitative ratio of water to alcohol, in particular polyol, [water:alcohol, in particular polyol] is in the range from 200:1 to 1:50, in particular in the range from 100:1 to 1:20, preferably in the range from 50:1 to 1:5, preferably in the range from 20:1 to 1:1, in particular with respect to the liquid medium, and/or is set.

In addition, the liquid medium may comprise an ether, in particular ether of a diol or polyol, preferably ether of a bicyclic diol, preferably dialkyl isosorbide, particularly preferably dimethyl isosorbide, particularly more preferably 2,5-dimethyl isosorbide, in particular in an amount in the range from 0.5 wt. % to 25 wt. %, preferably in the range from 1 wt. % to 10 wt. %, preferably in the range from 1 wt. % to 10 wt. %, based on the liquid medium.

In addition, the liquid medium may comprise at least one antioxidant, in particular ascorbic acid (vitamin C), in particular in an amount in the range from 0.1 wt. % to 10 wt. %, preferably in the range from 0.2 wt. % to 5 wt. %, preferably in the range from 0.5 wt. % to 2 wt. %, based on the liquid medium.

Furthermore, the liquid medium may comprise at least one biopolymer, in particular selected from the group of alginates, hyaluronic acid and chitosan (as well as their respective salts and derivatives) and mixtures and combinations thereof, in particular in an amount in the range from 0.1 wt. % to 50 wt. %, in particular in the range from 0.5 wt. % to 30 wt. %, preferably in the range from 1 wt. % to 10 wt. %, based on the liquid medium.

According to the invention, the carrier system or the liquid medium or the composition, in particular as defined above, is or are at least substantially free of aliphatic monoalcohols, in particular at least substantially free of aliphatic C1- to C4-monoalcohols, preferably at least substantially free of ethanol.

In addition, it is particularly the case here that the carrier system or the liquid medium or the composition, in particular as defined above, is or are at least substantially free of halogenated hydrocarbons, in particular at least substantially free of chlorinated hydrocarbons, preferably at least substantially free of chloroform.

The composition according to the invention is characterized by a high (storage) stability: Thus, the composition can be storage-stable in a temperature range of generally −20° C. to 45° C., in particular in a temperature range of −15° C. to 40° C., preferably in a temperature range of −15° C. to 20° C., preferably over a storage period of at least 4 weeks, in particular at least 6 weeks, preferably accompanied by particle sizes that at least essentially do not change over the storage period. The storage in particular can be determined under ambient pressure (1,013.25 hPa) and/or at a relative humidity of the ambient atmosphere of about 60%. The composition can be present in conventional preparation devices, such as glass vessels or ampoules.

In particular, with regard to the composition according to the invention, it is provided that the carrier system is in particulate form, in particular in the form of lipid nanoparticles (LNP), preferably lipid nanoparticles (LNP) in the form of micelles, vesicles and/or disk-shaped lipid nanoparticles (LNP) (disk lipid nanoparticles or disk LNP), disk-LNP), preferably disk-shaped lipid nanoparticles (LNP).

In particular, the carrier system and/or the composition, in particular the carrier system, may comprise a Log P value, in particular total Log P value, in the range from 0.1 to 5, in particular in the range from 0.2 to 4, preferably in the range from 0.3 to 3, particularly preferred in the range from 0.4 to 2. In particular, it may also be provided that the components and/or substances of the carrier system, in particular substances (i), (ii), (iii), (iv) and/or (v), are selected with the proviso that the resulting carrier system comprises a Log P value, in particular a total Log P value, of at most 5, in particular at most 4, preferably at most 3, particularly preferred at most 2, further preferred at most 1. Furthermore, it may be provided that the components and/or substances of the carrier system, in particular the substances (i), (ii), (iii), (iv) and/or (v), are selected with the proviso that the resulting carrier system comprises a Log P value, in particular a total Log P value, in the range from 0.1 to 5, in particular in the range from 0.2 to 4, preferably in the range from 0.3 to 3, particularly preferred in the range from 0.4 to 2.

With regard to further configurations of the composition according to the invention according to the present aspect, reference can be made to the configurations relating to the further aspects according to the invention, which apply accordingly in the present case.

Further subject-matter of the present invention—according to a further aspect of the present invention—is also the medicament or drug according to the invention, in particular for use in the prophylactic and/or therapeutic treatment of diseases of the human or animal body,
wherein the drug or medicament comprises or consists of at least one preferably lipid-based carrier system as defined above or a composition as defined above, in particular together with at least one pharmaceutically acceptable and/or pharmaceutically acceptable excipient.

According to the present aspect, the present invention also relates to a preferably lipid-based carrier system, as defined above, or composition, as defined above, for use in the prophylactic and/or therapeutic treatment of diseases of the human or animal body.

In this respect, the diseases can be selected from the group of bacterial, mycotic or viral, preferably viral, diseases, inflammatory diseases, diseases accompanied by fever or fever, allergic diseases, cancers, hormonal diseases, blood clotting disorders, cancers, deficiency states or metabolic diseases, skin diseases and combinations thereof.

In particular, the disease can be a viral infectious disease, in particular a disease caused by and/or coherent with coronaviruses, in particular an infectious disease, preferably a disease caused by and/or coherent with SARS-CoV-2, in particular an infectious disease, preferably COVID-19 (coronavirus disease 2019).

According to the present aspect, the present invention also relates to the previously defined drug or medicament or carrier system or composition for use in the prophylactic treatment of, in particular, a viral disease or infectious disease, in particular a disease or infectious disease caused by and/or coherent with coronaviruses, preferably a disease and/or infectious disease caused by and/or coherent with SARS-CoV-2, preferably COVID-19 (coronavirus disease 2019), infectious disease, preferably a disease and/or infectious disease caused by and/or in the context of SARS-CoV-2, preferably COVID-19 (coronavirus disease 2019), in particular wherein the drug or medicament or the carrier system or the composition is designed as a vaccine, preferably an mRNA vaccine.

The drug or medicament or carrier system or composition according to the invention may further be designed as an anti-cancer agent, in particular against skin cancer, preferably against white skin cancer. With regard to skin cancer, a topical application or administration may be considered or present in particular.

According to the present aspect, the present invention further relates to the use of the previously defined lipid-based carrier system according to the invention or the use of the previously defined composition according to the invention (for producing a medicament) or the use of the previously defined drug or medicament
for the prophylactic and/or therapeutic treatment of diseases of the human or animal body, wherein the diseases are selected from the group of bacterial, mycotic or viral, preferably viral, diseases, inflammatory diseases, diseases accompanied by fever or fever, allergic diseases, cancer diseases, hormonally induced diseases, blood coagulation disorders, cancer diseases, deficiency states or metabolic diseases, skin diseases and combinations thereof; and/or wherein the disease is a viral infectious disease, in particular a disease or infectious disease caused by and/or associated with coronaviruses, metabolic diseases, skin diseases and combinations thereof; and/or wherein the disease is a viral infectious disease, in particular a disease or infectious disease caused by and/or coherent with coronaviruses, preferably a disease or infectious disease caused by and/or coherent with SARS-CoV-2, preferably COVID-19 (coronavirus disease 2019), or
for the prophylactic treatment of, in particular, viral diseases or infectious diseases, in particular a disease and/or infectious disease caused by and/or coherent with coronaviruses, preferably a disease and/or infectious disease caused by and/or coherent with SARS-CoV-2, preferably COVID-19 (coronavirus disease 2019), in particular wherein the drug or medicament or the carrier system or the composition is designed as a vaccine, preferably an mRNA vaccine.

According to the present aspect, the present invention also relates to a method for treating a disease of the human or animal body, comprising the step of administering an effective, in particular prophylactically and/or therapeutically effective, amount of the previously defined carrier system according to the invention or of the previously defined composition according to the invention or of the previously defined drug or medicament according to the invention,
in particular wherein the disease is selected from the group of bacterial, mycotic or viral, preferably viral, diseases, inflammatory diseases, diseases accompanied by fever or fever, allergic diseases, cancers, hormonal diseases, blood coagulation disorders, cancers, deficiency states or metabolic diseases, skin diseases and combinations thereof; and/or in particular for the treatment of a viral infectious disease, in particular a disease or infectious disease caused by coronaviruses and/or associated therewith metabolic diseases, skin diseases and combinations thereof; and/or in particular for the treatment of a viral infectious disease, especially a disease or infectious disease caused by and/or coherent with coronaviruses, preferably a disease or infectious disease caused by and/or coherent with SARS-CoV-2, preferably COVID-19 (coronavirus disease 2019).

In the context of the present invention, the drug or medicament or carrier system or composition may be applied topically or systemically, in particular systemically. Accordingly, the drug or medicament or carrier system or composition may be prepared for topical or systemic, in particular systemic, application.

In particular, the drug or medicament or the carrier system or the composition can be applied systemically, in particular parenterally or perorally, preferably parenterally, such as intravenously, intraarterially, intramuscularly, subcutaneously or the like, preferably intramuscularly. In this respect, the drug or medicament or the carrier system or the composition can be prepared for systemic, in particular parenteral or peroral, application, preferably parenteral application, such as intravenous, intra-arterial, intramuscular, subcutaneous application or the like, preferably intramuscular application.

In particular, the drug or medicament or the carrier system or the composition can be applied topically, in particular cutaneously. In this respect, the drug or medicament or the carrier system or the composition may be prepared for topical, in particular cutaneous, application.

In addition, the carrier system or the composition can be used as a disinfectant, in particular a surface disinfectant, in particular with the use of ingredients or active ingredients with a disinfecting effect. In particular, glycerol monolaurate can also be used for this purpose.

For further configurations of the subject-matter according to the invention according to this aspect, reference can also be made to the configurations of the other aspects according to the invention, which apply accordingly.

A further subject-matter of the present invention—according to a further aspect of the present invention—is the use of at least one process parameter for controlling, in particular for method and/or process control, the method defined herein, wherein the process parameter is selected from the group of temperature, conductivity, viscosity, pH value, electrical resistance and surface tension, in particular dynamic surface tension, preferably from the group of temperature, conductivity, viscosity and pH value, in particular in each case of the liquid medium, pH value, electrical resistance and surface tension, in particular dynamic surface tension, preferably from the group of temperature, conductivity, viscosity and pH value, in particular in each case of the liquid medium, preferably together with the components and/or substances introduced therein, and/or the composition, as defined above.

For further configurations of the subject-matter according to the invention according to this aspect, reference can also be made to the configurations of the other aspects according to the invention, which apply accordingly

Further advantageous properties, aspects and features of the present invention are also apparent from the figures cited and their description, as will also be given in detail below.

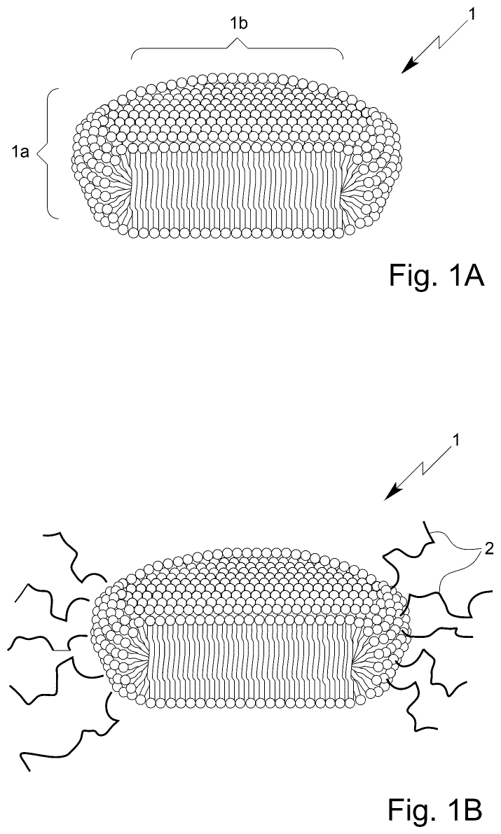

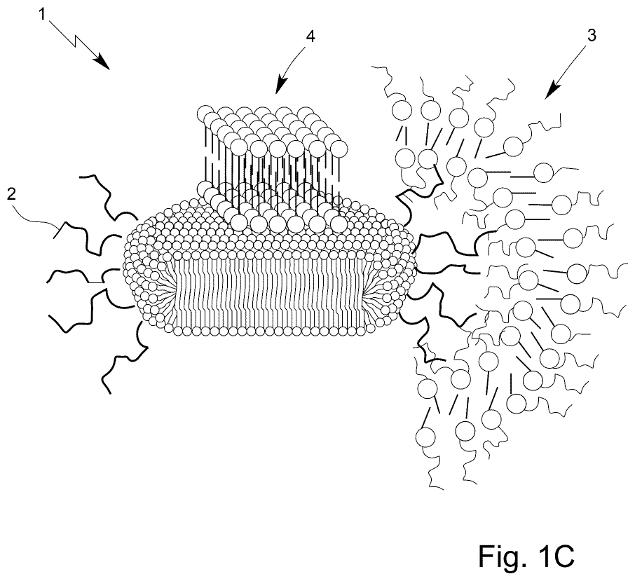

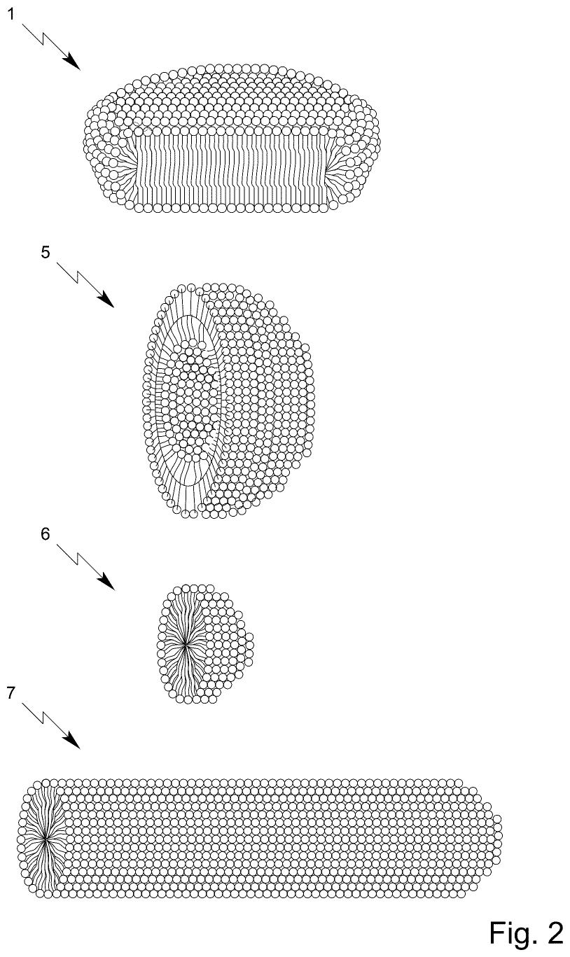

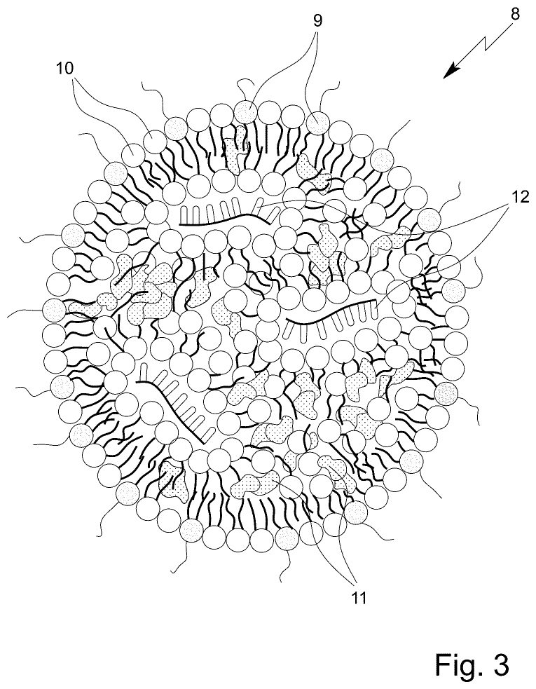

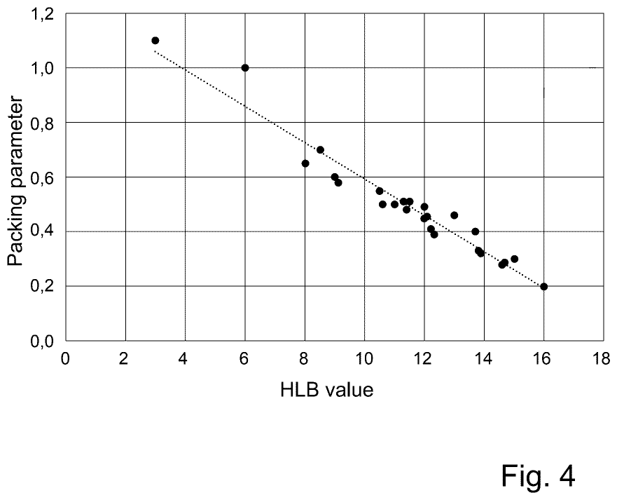

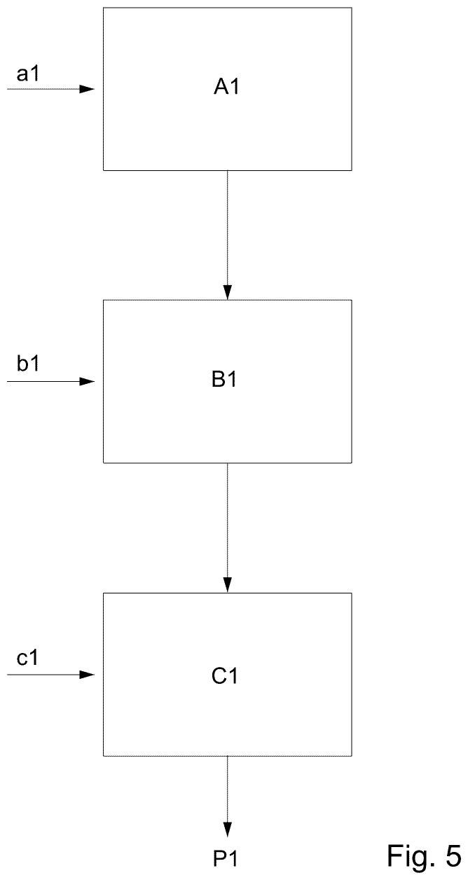

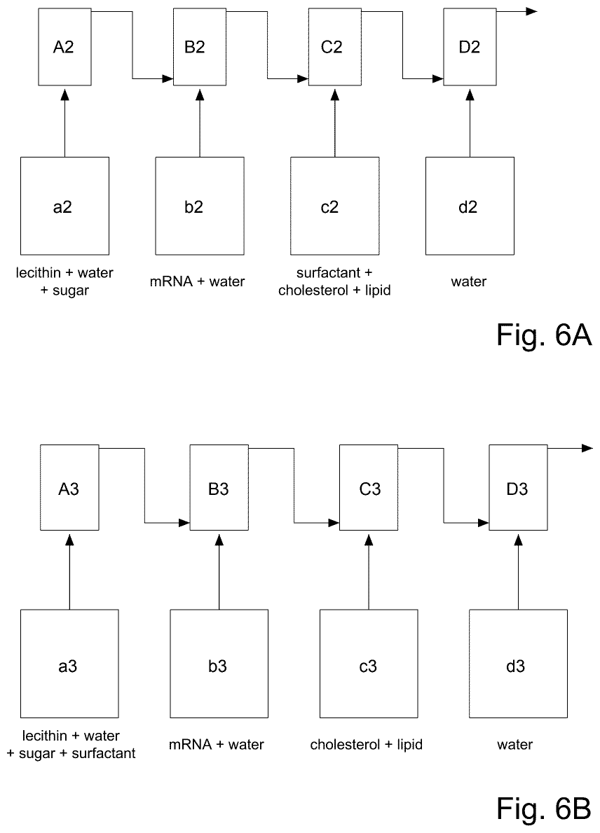

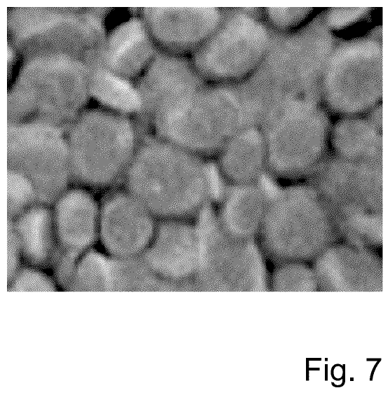

The figures show:
FIG. 1A a schematic and illustrative representation of the structure or shape of a particle of the carrier system according to the invention in the form of a disc-shaped lipid nanoparticle 1 (also known as a disc lipid nanoparticle (disc-LNP), disc vesicle or disk-lamella); the disk-shaped lipid nanoparticle comprises in particular a cylindrical or disk-shaped geometry with a peripheral region or a lateral surface 1a and in particular circular or in particular circularly shaped base surfaces 1b
FIG. 1B a schematic and illustrative representation of a disc-shaped lipid nanoparticle 1, according to which the surface area or the edge region has non-ionic amphiphilic or surfactant substances, in particular PEGylated substances or PEGylated surfactants 2 (see substance (ii)), while the shell surfaces comprise mainly phospholipids (see substance (i)); in this context, the underlying HLB value according to Griffin or the respective critical packing parameter is also relevant for the arrangement of the substances in the disc-shaped lipid nanoparticle, according to which substances with a relatively high Griffin HLB value or a relatively low critical packing parameter are primarily arranged in the peripheral area or in the area of the shell surface, while substances with a relatively low Griffin HLB value or a relatively high critical packing parameter are primarily arranged in the area of the base surfaces;
FIG. 1C a schematic and illustrative representation of a disc-shaped lipid nanoparticle 1 of the carrier system according to the invention, which in the peripheral region likewise comprises the substances 2 mentioned above in FIG. 1B and according to which the lipid nanoparticle is multilayer, in particular on the basis of further phospholipids 4, which are arranged in particular parallel to the base surfaces of
   the underlying disc-shaped structure, and further substances 3, which are arranged in the shell region and in particular can interact with the substance 2; the particles according to FIG. 1A to FIG. 1C are shown in particular for purposes of illustration as a perspective sectional view, wherein the respective circular representations illustrate the hydrophilic regions and the respective line representations illustrate the lipophilic regions of the underlying molecules;
FIG. 2 a schematic and illustrative representation of the formation of the carrier system particles according to the invention on the basis of the respective basic structures in the form of a disc-shaped lipid nanoparticle 1; FIG. 2 also shows further, although less preferably, configurations of the particles, namely in the form of a vesicle 5, a spherical micelle 6 and a worm-like micelle 7; the particles 1, 5, 6 and 7 are in particular illustrated in the form of a sectional representation, wherein the respective circular representations and the respective line representations in particular represent the lipophilic region of the underlying molecules;
FIG. 3 a schematic and illustrative representation of another particulate form or particle 8 with components based on PEGylated substances 9, phospholipids 10, cholesterol 11 and active ingredient 12;
FIG. 4 a graphical representation in the form of a diagram to illustrate the relationship between the packaging parameter Pₖᵣ on the one hand and the HLB value according to Griffin on the other;
FIG. 5 a schematic representation of a method of operation according to the invention based on respective batches or a divided supply of the ingredients or components a1 to c1 in respectively assigned receiving or processing devices A1 to C1 in the context of a continuous or cascaded and/or consecutive process operation for obtaining the composition;
FIG. 6A a schematic representation of a method according to the invention with an exemplary division of the substances of the carrier system or the components for the composition to be produced for the purpose of forming corresponding batches or starting from respective storage devices a2 to d2 and with processing in corresponding receiving devices (A2 to D2) in the context of a continuous and in particular cascaded or consecutive process operation with successive combining of the batches according to an embodiment of the invention;
FIG. 6B a schematic representation based on FIG. 6A according to a further embodiment of the invention with an alternative distribution of the substances or components; the reference signs a3 to d3 correspond to a2 to d2 according to FIG. 6A, wherein the same applies to A3 to D3 and A2 to D3;
FIG. 7 a cryo-scanning electron microscope image of an invention-composition with the invention-carrier system based on particles in the form of disk-shaped lipid nanoparticles; the average particle diameter is 150 nm±70 nm; polysorbate 20 and retinol are used as amphiphilic main components;
FIG. 8 a graphical representation in the form of a diagram to illustrate the relationship between the HLB value according to Griffin on the one hand and the Log P value on the other hand (in semi-logarithmic coordinates) as shown in the context of the invention.

Further configurations, modifications and variations, as well as the advantages of the present invention, are readily recognizable and realizable for the expert when reading the description, without him thereby leaving the scope of the present invention.

The method according to the invention also enables standardized industrial production, including with regard to (process) automation and (process) digitization. In particular, the present invention also fulfills essential criteria and requirements, as summarized under the term “Industry 4.0”. The term “Industry 4.0” goes back to the research union of the German federal government and a project of the same name in the high-tech strategy of the federal government. In this context, the present invention can, for example, be carried out using modern information and communication technology based on intelligent and digitally networked systems, wherein self-organized production is also possible, wherein the entire value chain can also be optimized.

In particular, the present invention enables flexible production: for producing and developing the underlying products, digital networking can be used to coordinate the respective process steps well with one another and, overall, to better plan and optimize the utilization of the production facilities used in the process. In addition, the criterion of the so-called convertible factory is also met. Production lines can be built in modules and independently of their size or capacity, wherein a fast and uncomplicated construction or provision with regard to the objects to be used is made possible. In particular, improved productivity or cost-effectiveness can also be provided, wherein individualized products can also be produced in small quantities at a reduced cost. In addition, customer-oriented or customer-centered solutions can also be provided, wherein the producer on the one hand and the customer or buyer on the other hand move closer together, especially since customers or buyers can help to design products according to their requirements or an adaptation of corresponding products to the individual requirement profile of the respective customers or buyers is possible. In addition, optimized logistics are also available, especially since the compositions according to the invention or LNP products can be produced globally or, so to speak, independently of location. With regard to the present invention, a focused use of data is also possible. In this way, data on the production process and the condition of corresponding products can be acquired, combined and evaluated. In this context, data analysis can also be used to determine how a product can be produced with even greater efficiency or with improved properties. The corresponding data can also serve as a basis for further business models and services.

With regard to the aforementioned criteria, the following is relevant to the present invention in summary and in addition:
Flexible production: The carrier systems provided according to the invention, preferably in the form of lipid nanoparticles (LNP), or the corresponding compositions can be individually designed or customized by using the corresponding HLB values or Log P values of the components or substances used for this purpose, wherein a system is provided that is calculable or individually adaptable overall, also with regard to the individual configuration and shaping of the lipid nanoparticles. In particular, this approach can be used to quickly and reliably develop and provide both liposome LNPs and disc LNPs, taking into account the respective requirements and areas of application. In addition, critical process parameters can be determined and optimized during the development phase. In addition, continuous control of the underlying process parameters is possible throughout the entire manufacturing process, wherein in particular continuous method and process and production control, in particular online or inline process control, can be carried out.
Convertible factory: The special process operation according to the invention enables improved productivity and cost-effectiveness, wherein individualized or customized products can also be provided in small amounts or quantities with optimized cost efficiency.
Optimized logistics: Based on the method according to the invention, the carrier systems or compositions provided within the scope of the present invention can be produced globally or, so to speak, independently of location. In particular, there is also optimized energy efficiency. The low-energy production enables the use of solar energy, for example. This enables, for example, the efficient production of so-called coating applications on site.

The above explanations show the overall advantages and properties associated with the present invention.

The following examples are only intended to illustrate the present invention, but are not intended to limit the present invention to them. In this context, further general aspects of the present invention are also mentioned below, which further characterize the present invention, independently of the examples given.

### Examples and Further General Explanations of the Present Invention

The following examples illustrate the advantages and special features and characteristics as they are associated with the concept according to the invention. In the following, supplementary or further features, properties and advantages of the present invention are also shown, which further characterize the following invention, also independently of the embodiment examples given.

In general, the embodiments and investigations described below are carried out on a pilot plant or laboratory scale. With regard to the formation of corresponding carrier systems, the procedure is generally carried out with the introduction of energy or mixing or stirring energy, namely in such a way that the energy introduced is set in such a way that an at least essentially laminar flow is set in the underlying medium. In addition, continuous method or process control is generally carried out in particular, acquiring relevant process parameters such as temperature and conductivity. Based on the acquisition of the process parameters, the process parameters can be adapted or adjusted accordingly to form optimal compositions or carrier systems.

### Part A:

2,5-Dimethylisosorbide (DMI) is a clear, colorless liquid with a faint odor that mixes with water and many organic solvents. As a bio-based ether, DMI comprises generally favorable properties (e.g. low eye and skin irritation and very low toxicity). As a low-toxicity solvent, dimethyl isosorbide is also suitable for pharmacologically active substances whose penetration capacity into the skin, for example, is to be promoted.

In particular, the dissolving properties towards relatively high-melting substances, such as cholesterol, phytosterol or distearoylphosphatidylcholine, at relatively low temperatures and high concentrations of the substances to be dissolved are also surprising. This behavior is tested in detail using phytosterol as an example.

The hydrophobic phytosterol stigmasterol, whose melting point is around 170° C., can be completely dissolved in the hydrophilic 2,5-dimethylisosorbide, in particular in the temperature range from 55° C. to 70° C., even in concentrations of over 50 percent. In the aforementioned temperature range, cholesterol and phosphatidylcholines that melt at high temperatures can also be dissolved in high concentrations or amounts.

2,5-Dimethylisosorbide promotes the production of LNP and leads to a very small particle size of the resulting lipid nanoparticles, even if it is added or used in relatively high concentrations, as the following example according to Table 1 shows.

TABLE 1

| 1st batch (phase A) | |
|---|---|
| 2,5-Dmethyl isosorbide (DMI) [wt. %] | 20.0 |
| Phytosterol [wt. %] | 3.7 |
| Phosphatidylcholine [wt. %] | 6.5 |
| PEG 40 Stearate [wt. %] | 14.1 |
| Capric acid/caprylic acid | 22.2 |
| triglyceride (CCT) [wt. %] | |
| 2nd batch (phase B) | |
| Water [wt. %] | 33.4 |

When producing this LNP emulsion, all components of phase A are first added to a stirring vessel and then heated to 55° C. This produces a homogeneous, clear solution. This produces a homogeneous clear solution. The water according to phase B, which comprises a temperature of approx. 20° C., is added to this solution while stirring, wherein in particular a liquid-crystalline gel or a carrier system based on defined lipid nanoparticles present in the liquid medium is formed. The obtainable composition can be diluted with water as desired to obtain the respective concentrations. The particle size of the carrier system based on lipid nanoparticles LNP is determined using laser diffractometry. A value of 150 nm+40 nm is determined. Due to the low melting point of −70° C., the corresponding particle-containing compositions containing 2,5-dimethylisosorbide can also be stored at low temperatures without negatively influencing the particle structure or the underlying dispersion. The average HLB value according to Griffin of the amphiphilic surfactant components used in the first preparation or in phase A and the PEGylated surfactant is HLB=6.4. In addition, there is a Log P value of about 1.03.

### Part B:

### Compositions Based on Phosphatidylcholine, Pegylated Surfactant and Glycerol Mono Laurate

Glycerol monolaurate also comprises germ-reducing, antimicrobial and antibacterial properties, wherein it is very effective against Helicobacter pylori in particular. In particular, lipid nanoparticles which are equipped or loaded with glycerol monolaurate as an encapsulated component in particular can comprise an antimicrobial spectrum.

Infections with Helicobacter pylori are held responsible for a number of gastric diseases which are associated with an increased secretion of gastric acid. The lipid nanoparticles according to the invention, which are based on phosphatidylcholine and glycerol monolaurate, also comprise the advantage that they can also reduce the concentration of gastric acid by protonating phosphatidylcholine.

For an underlying oral application, the lipid nanoparticles or the particle system or the composition containing the lipid nanoparticles according to the invention can be provided with sorbitol and/or a sweetener, such as acesulfame, in particular to improve the taste during oral application, in particular any bitter taste can be compensated by sweetness (i.e. addition of sweeteners in particular).

In this context, the compositions listed below in Tables 2 and 3 are produced with the specified properties. The mean HLB value according to Griffin given in the tables refers to the mean value of the components or substances used for the carrier system. The same applies to the mean Log P value and thus the total Log P value.

If the compositions based on phosphatidylcholine, pegylated surfactant and glycerol monolaurate are used and a short-chain glycerol caprylate or sorbitan caprylate is added to these, an excellently effective skin disinfectant or surface disinfectant can also be obtained due to the broad antimicrobial and antibacterial spectrum. Such compositions show excellent film formation after application or application and can therefore at least essentially completely cover the surfaces to be treated. Comparable properties are obtained in alternative compositions or formulations with a (further) active ingredient (antimicrobial active ingredient or vaccine active ingredient (mRNA)).

TABLE 2

| | | |
|---|---|---|
| Phosphatidylcholine [wt. %] | 3.5 | 3.1 |
| PEG 40 stearate [wt. %] | 7.0 | 11.0 |
| Glycerol monolaurates [wt. %] | 5.2 | 5.5 |
| Phytosterol [wt. %] | 2.2 | 2.3 |
| Capric acid/caprylic acid | 9.2 | 9.1 |
| triglyceride [wt. %] | | |
| Glycerol [wt. %] | 16.0 | 16.8 |
| Water [wt. %] | 57.0 | 52.3 |
| | 100.00 | 100.00 |
| Particle size [nm] | 1.200 ± 300 | 620 ± 160 |
| medium HLB according to | 6.4 | 7.8 |
| Griffin | | |
| mean log P-value | 1.03 | 0.82 |

TABLE 3

| | |
|---|---|
| Phosphatidylcholine [wt. %] | 1.9 |
| PEG 40 stearate [wt. %] | 7.8 |
| Glycerol monolaurate [wt. %] | 8.4 |
| Sorbitol caprylate | 3.6 |
| Capric acid/caprylic acid | 10.7 |
| triglyceride [wt. %] | |
| Glycerol [wt. %] | 3.8 |
| Water [wt. %] | 63.8 |
| | 100.00 |
| Particle size [nm] | 750 ± 200 |
| medium HLB according to | 7 |
| Griffin | |
| mean log P-value | 0.93 |

### Particle Size as a Characteristic of the Carrier Systems According to the Invention, in Particular in the Form of Disk-Shaped Lipid Nanoparticles (Disk-LNP)

The following configurations are based on producing, using and formulating carrier systems according to the invention in the form of disc-shaped lipid nanoparticles, wherein the carrier system is based on phosphatidylcholine and a pegylated surfactant as well as amphiphilic substances whose joint mean HLB according to Griffin is less than 8, and optionally an active ingredient. The size of the disc-shaped lipid nanoparticles is defined in particular by a Gaussian distribution with a variance in the range of ±20-35% around the mean value (Table 4).

TABLE 4

| Particle size | Stirring time | Variance | Variance |
|---|---|---|---|
| [nm] | [s] | [±] | [%] |
| 1.900 | 30 | 0.6 | 31.6 |
| 1.400 | 60 | 0.38 | 27.1 |
| 1.200 | 90 | 0.33 | 27.5 |
| 1.000 | 120 | 0.25 | 25.0 |
| 900 | 150 | 0.2 | 22.2 |
| 750 | 180 | 0.16 | 21.3 |

In particular, both the particle size, especially the median particle size, of the particles of the carrier system and the variance can be controlled via the stirring duration or time of the coherent liquid crystalline structure to be formed (duration of the input of mixing or stirring energy). In particular, the median particle size decreases exponentially with increasing stirring time. In addition, the percentage variance also decreases, in particular on a logarithmic scale. This behavior can be seen as a characteristic or special property of the particle system according to the invention, in particular in the form of disk LNPs. The spectrum or the values of the average HLB value according to Griffin for combinations based on pegylated surfactants and phosphatidylcholine generally shifts towards higher values due to the addition of non-ionic substances comprising a higher HLB value than the phosphatidylcholine used. The Log P value can shift towards lower values accordingly.

### Formation of Particles in the Form of Disk-Shaped Lipid Nanoparticles in Relation to the HLB Value According to Griffin

As already described and as surprisingly found by the applicant, it is advantageous according to the invention with regard to the formation of disk LNP in particular (cf. also FIGS. 1A, 1B and 1C and FIG. 2 (reference sign 1)) if an average HLB value according to Griffin in the range from 2 to 8 is selected for the entirety of pegylated surfactants and amphiphilic substances of the carrier system. What is certainly surprising here is that in the prior art the HLB range of 3 to 8 is generally assigned to W/O emulsifiers.

### Part C:

### Objective

Structured dosage forms, such as liposomes and lipid-based particles, offer new therapeutic options, as has also become apparent in the rapid development of Covid-19 vaccines. The first liposomal finished medicinal products were introduced into therapy more than 20 years ago, but only a few other products have reached market maturity in the years since.

As in biological membranes, the structure of the particles can generally be formed by phospholipids with cholesterol embedded in them. The lipid particles can also contain other lipid components that impart special properties.

Liposomes have been used as carriers for drugs in medicine for over 20 years. In another approved drug, therapeutic RNA molecules are packaged in lipid particles (see Onpattro®). In comparison to vaccination, significantly higher quantities of lipid are administered intravenously in these drugs. Lipid particles are also used as carriers for mRNA and ensure that the mRNA used can be transported more easily into the body's cells. There, it triggers various reactions, resulting in immunization against the SARS-CoV-2 virus, for example.

Lipid particles, and in particular with regard to lipid (nano)particles, especially if they serve as carriers for mRNA, are at best very complex to produce. In particular, the prior art often results in relatively large particles that are not homogeneous, in particular with regard to their particle size distribution, and often with excessively large dispersity of the underlying particles. The resulting dispersions are often not sufficiently stable and can only be stored with great effort, in particular at very low temperatures.

For medical applications, the focus is on vesicle structures or particles. These structures mostly consist of phosphatidylcholines and cholesterol. The procedure for producing them is often such that a carrier oil, phospholipid and any other hydrophobic components are dissolved in a solvent, often ethanol, which must then be removed again. In addition, the addition of buffer solution and an active ingredient (for example an antigen or an mRNA coding for an antigen in the case of vaccines) is effected, wherein in particular large multilayer or multilamellar vesicles (MLVs) are formed, which are then processed into smaller vesicles in a Microfluidizer®-high-pressure homogenizer. If the carrier material is liquid or can be melted in water, the hydrophobic components can also simply be mixed and added to the aqueous phase before the material is processed in the homogenizer.

Producing them is therefore very time-consuming and does not always result in the desired product quality. Such a process is not particularly suitable for consistent batch/batch quality. In general, the LNP structures as carriers of the mRNA should be based on vesicles. Due to the ratios of the pegylated substances to the water-insoluble amphiphilic substances present in the carrier of the vaccine, a homogeneous vesicle structure is generally not given, especially since the present packing parameters of the substances involved are opposed to such a structure or do not allow such a structure. This also leads to short shelf lives or storage periods. For example, mRNA vaccines must be used within short periods of time. If this period is exceeded, the physico-chemical stability of the product is no longer sufficiently ensured. In contrast, the desired presence of the lipid particles in a homogeneous form can lead to greater stability and stability over a much longer period.

The need for strong cooling, especially of mRNA vaccines, for example to a temperature of −20° C. to −70° C., also represents an almost unmanageable difficulty, in particular during a pandemic, especially since not all regions worldwide can establish the necessary cold chains.

The extremely complex production process can also represent an obstacle to the distribution of the vaccine in certain regions. Equipping the lipid particles with hydrophilic antigens, for example, which is effected on their surface after producing them, is also not always optimal, in particular as the underlying particles sometimes do not comprise a surface size that is optimized in terms of volume.

In addition, the manufacturing methods of the state of the art can often only be realized on a small scale.

### Solution and Further Configurations

The solution to the problem should enable simple producing, which on the one hand guarantees high batch quality and on the other hand opens up the possibility of producing lipid nanoparticles (LNP) on a large scale and in consistent quality with defined size properties.

Lipid nanoparticles (LNP) are particles whose structure is composed of lyotropic liquid-crystalline regions and whose size is generally in the scaled nanoscale, as also mentioned above. Coherent liquid-crystalline structures belong to self-organized systems and are thermodynamically stable, without wanting to refer to or limit themselves to this theory.

They therefore form defined structures under given external conditions and therefore also lead to a consistent quality. Both water-soluble and inverse, i.e. oil-soluble, structures are known. Water-soluble structures are of particular importance for the formulation of pharmaceutical carrier systems.

Lyotropic liquid-crystalline water-soluble structures can be present in three main types, namely in a lamellar form, which is in particular homogeneous and/or in particular coherent, in a hexagonal or in a cubic form. These lyotropic liquid crystalline forms can in principle comprise further substructures. The three structures are also characterized in particular by the fact that their compartments form a coherent structure if necessary. The structures of the laminar and hexagonal phase appear milky and represent gels with viscoelastic behavior.

The cubic phase is generally transparent. A prerequisite for the production of a cubic structure is that the particles are spherical or ellipsoidal. A special form of the cubic structure is the formation of vesicles whose structure consists of lamellae. Liquid crystalline lamellar structures show a number of substructures. Of these substructures, the structure consisting of individual disk compartments is of importance for the formation of LNPs, which serve as carriers for active substances in pharmaceutical applications and which can be provided according to the invention in a targeted and purpose-oriented manner. Lyotropic liquid-crystalline phases can form a dispersion based on the compartments from which they are composed when diluted with aqueous phases. This process is reversible. If water is evaporated from LNP dispersions formed via lyotropic liquid-crystalline systems, the originally coherent phase can form back when the critical water concentration is reached.

The lyotropic liquid-crystalline structures can be highly diluted with water. The water used for dilution may also contain electrolytes or other non-surfactant water-soluble substances. When diluting the lyotropic liquid-crystalline phase, the viscosity decreases exponentially after the critical dilution concentration and the water activity increases exponentially. The lyotropic liquid-crystalline structures are not determined by a specific water concentration, but are stable over a concentration range of the bound water, depending on the structure. The concentration range of the necessary water in an already formed liquid-crystalline structure can be several percent by weight.

During the production of liquid crystalline structures, the water present is highly bound. The degree to which the water is bound can be calculated using the water activity. The determination of water activity is already an established method that is often used as a substitute for a conservation load test (KBT). Microorganisms no longer multiply if the water activity is below the value of 0.5. The determination of water activity as a CBT is defined in the ISO 29621 standard. Today, the determination of water activity is an important tool for the quality control of products and ingredients in the food, pharmaceutical and cosmetics industries. The measurement of water activity is effected according to ISO 21807 and ISO 18787+21 CFR Part 11, which is also defined in the ISO 29621 standard. Water activity also influences the course of chemical processes such as fat oxidation.

In particular, a water concentration is present at which the structure irreversibly breaks down into its individual compartments and forms an aqueous dispersion.

Lytropic liquid-crystalline structures are stable within a temperature window and can, in contrast, disintegrate irreversibly, usually when stored below or above the critical temperature.

A specific liquid-crystalline structure to be formed depends, as surprisingly found, in particular also on the packing parameter of the substances involved. According to the Israelachvili model, the critical packing parameter Pₖᵣ is based on the formula Pₖᵣ=V/a₀−l_{c}, in particular as previously configured. Pₖᵣ describes, similar to the HLB value, a ratio of hydrophilic head groups to the lipophilic groups of an in particular amphiphilic substance or compound, wherein the focus is on geometric and space-filling components. The Pₖᵣ is dimensionless and characteristic for each compound used. Geometric shapes of lyotropic liquid-crystalline structures can be controlled or predicted using the Pₖᵣ in particular.

The packing parameter of an amphiphilic substance or compound in particular can also be estimated via its HLB value according to Griffin. If, for example, the Pₖᵣ value of fatty alcohols with different C-chain lengths and different degrees of ethoxylation is calculated, these values correlate very well with the HLB values with R²=0.94 (see also FIG. 4). These values can be transferred to other substances accordingly. In addition, the packing parameter can also be determined on the basis of the relevant log P values.

Since, if the HLB value of each individual substance is known for a mixture of substances, in particular of surfactant or amphiphilic substances, the average HLB value can be calculated, it is also possible to assign an average packing parameter to the mixture as such due to the relationship between the HLB value and the packing parameter Pₖᵣ. In this way, a control or estimation of the liquid-crystalline crystal structure formed from the given mixture can be performed in a surprising way.

In principle, the log P value can also be used in this regard, which is equally surprising.

The relationship between the packing parameter of an in particular non-ionogenic substance and its HLB value can be approximated mathematically as follows: Pₖᵣ=0.07. HLB+1.26 (where Pₖᵣ=critical packing parameter; HLB=HLB value of a substance, in particular an amphiphilic or surfactant substance, or a mixture thereof; see FIG. 4). The HLB value of a substance is therefore directly proportional to the critical packing parameter and can also be determined on this basis.

Griffin set an HLB system for non-ionic or, in particular, surface-active compounds, which assigns numerical, dimensionless values between 0-20 to the substances or compounds according to the weight ratio of the hydrophilic molecular component to the total molecule and thus allows statements to be made about the water solubility of the substance.

As previously mentioned, the formula HLB=20·M_{hydrophilic}/Mₜₒₜₐₗ is used as the basis for calculation. An average HLB value according to Griffin can be calculated from combinations of several substances, in particular amphiphilic substances.

The behavior of an amphiphilic or substance can be influenced by parameters such as the pH value. For non-ionic surfactants, their surface-active properties can sometimes change with regard to the temperature present. For non-ionic surfactants, the influence of the electrolyte is moderate.

The cloud point is the temperature at which a non-ionic surfactant solution becomes cloudy. About 0.5° C. above the cloud point, the surfactant is completely insoluble in water. The higher the HLB value (or the lower the Log P value), the higher the cloud point. The cloud point is therefore a characteristic feature of the respective surfactant and its area of application. The cloud point depends on the concentration of the electrolytes.

In the case of ionic or amphoteric surfactants or lipids, the HLB value can be influenced by the pH value. In this case, similarities to non-ionic substances in their behavior in aqueous media can be used.

For ionic, in particular cationic, and/or amphoteric substances, the HLB value can be determined within the scope of the present invention, in particular for the uncharged state or at the isoelectric point. In addition, the HLB value can be determined according to the invention, in particular at room temperature (20° C.).

For the phosphatidylcholine distearoylphophatidylcholine (DSPC), a value of HLB of 4.5 is given in the literature (which corresponds to a Log P value of 1.54). This value is also consistent with the derived packing parameter.

Amphoteric surfactants or lipids, such as phospholipids, lose their water solubility in proximity to the isoelectric point and transform into co-surfactants. In this area, phospholipids behave similarly to non-ionic surfactants. If an HLB value is calculated for phospholipids according to Griffin, this corresponds to the value at the isoelectric point. Below and above the isoelectric point, the calculated HLB value increases.

### Influence of the Production of Lamellar Lyotropic Liquid Crystalline Structures on LNP Formation

One possible packing parameter for the production of lamellar liquid crystalline structures is in particular in the range Pₖᵣ=0.5, which corresponds to an HLB value range according to Griffin of HLB=10 to 12 or a Log P value range of Log P=0.50 to 0.62. In this HLB range, vesicle-like structures can be formed at first, which may comprise a cubic shape in particular. The cubic vesicle structure may in particular be transparent. If this form is diluted with water, spherical LNPs are formed in particular. In principle, hexagonal spherical packings of vesicles are also possible.

If a PEGylated surfactant is used for the production of these phases, it is evenly distributed over the entire lamella of the vesicle. Vesicles formed with PEGylated substances that are distributed over the entire surface of the particle are only suitable to a limited extent as carriers for active substances that are to be introduced into a human cell, as the PEG residues of the pegylated substance also form a steric barrier with or towards the cell.

The liquid crystalline gel structures with an average HLB value of 11.7 and 10.1 (in particular log P values of 0.52 and 0.61) represent in particular clear gels, which can be assigned to the cubic structure. These are spherical or ellipsoidal vesicle structures.

In particular, a substructure of the vesicle-like liquid-crystalline lamellar structure is formed by the disk structure or disk LNP, which is formed in particular by a surfactant or (phospho) lipid with an HLB value calculated according to Griffin of at least 13 (in particular a Log P value of at most 0.46) and in particular with preferably non-water-soluble amphiphilic substances with an HLB of at most 6 (in particular a Log P value of at least 1.11). The HLB value averaged from the HLB values of the surfactants or lipids with an HLB of at least 13 and the non-water-soluble amphiphilic substances with an HLB of at most 6 is in this respect preferably an HLB of at most 5 (in particular a Log P value of at least 1.36).

Liquid crystalline or liquid-crystalline disk structures have a cylindrical geometry. In this geometry, the outer surface is primarily or predominantly covered with PEGylated surfactants, for example, in accordance with the law of packing parameters, while the circular base surfaces are covered with the non-water-soluble amphiphilic components. An average HLB value according to Griffin, which describes the conditions for the composition of the amphiphilic components for the production of disk structures, is not yet known, and it is completely surprising that this can be used with regard to the production of disk-shaped LNPs. The same applies to the log P value.

### Producing LNP Structures

Input of Mixing and/or Stirring Energy

According to the invention, the (self-) organization of lyotropic liquid-crystalline structures from an amphiphilic mixture is effected by the introduction of mixing or stirring energy. On this basis, the structuring or organization of the particulate structures is induced or caused or, so to speak, catalyzed (“catalysis via mixing or stirring energy”). In order to achieve the production of the desired structure, the particles are thus pushed into a position favorable for the production of liquid crystalline structures. According to the invention, it has proven to be surprisingly advantageous if the energy produced or introduced by the stirring is in the laminar flow range. Turbulent flows disrupt the desired self-organization. The high energy introduced in this case W can become significantly too high (U=Q+W; U can therefore assume significantly too high values).

According to the invention, the stirring energy introduced for the production of liquid crystalline structures is in particular only in the laminar flow range. The laminar flow behavior is described by the Reynolds number Re, as mentioned above.

In particular, there may be a transition from laminar flow to turbulent flow at R_{e,krit} above 2,000, in particular at R_{e,krit}=2,200 to 2,300, which should be avoided according to the invention (Rₑ>R_{e,krit}=>flow is turbulent Rₑ<R_{e,krit}=>flow is laminar). To maintain the laminar flow, conventional stirring tools or devices can be considered, which in particular produce at least essentially no or at most only slight local heat generation during use.

### Use of Polyols

Surprisingly, the addition of polyols to the water phase or the liquid medium used for the production of the liquid-crystalline phases has a significant influence on the particle size.

Within a critical concentration range of the polyol in the water phase, significantly smaller particles are formed than in pure water phases. A preferably polyol concentration in the water phase is between 10 and 60 percent, based on the water phase. The particularly preferred range depends in particular on the structure of the polyol used. Polyols such as propylene glycol, glycerol, polyethylene glycol, sorbitol, mannitol, sucrose or panthenol are particularly suitable.

Panthenol in particular can exhibit multifunctional properties in LNP formulations due to its structure. Due to the aminopropanol group present, which is similar to the amphiphilic substances used in mRNA vaccines, panthenol is a water-soluble substance at physiological pH, but has been shown to anchor in or on lamellar structures. Panthenol also promotes penetration behavior, which has been demonstrated in the literature in several examples, including progesterone.

The reason for the improved formulation of the liquid-crystalline disk structure appears to be due to the reduced water activity of polyol solutions. A water activity of 0.9 to 4.5 appears to be optimal for the production of LNP for the polyol solutions used for this purpose.

Since each polyol that is mixed with water leads to a specific reduction in water activity, the optimum concentration range for the production of optimum conditions, in particular with regard to a specific reduction in water activity, can be set depending on the specific polyol used. In terms of water activity, a possible range for aqueous sucrose is in the aforementioned range.

In this context, corresponding compositions based on the carrier system and the liquid medium are produced in the context of the present inventions, namely with respect to the liquid (aqueous) medium (1st batch or phase B) on the one hand with the use of sucrose as a polyol (12 wt. %, based on the composition) and on the other hand without sucrose (replacement by water). The components of the carrier system are specified as the first batch or phase A. The preparations are combined to obtain the composition. An average particle size of 590 nm±170 nm can be determined for the composition containing sucrose compared to 1,500 nm±350 nm for the composition without sucrose.

### Influence of the Mean HLB Value or Mean Log P Value on the Particle Size of Disk LNP Structures

The average HLB value according to Griffin, which is calculated from the HLB values of the hydrophobic amphiphilic components and the pegylated surfactants, also plays an important role in the production of stable disk LNPs. The same applies to the corresponding Log P value. In order to produce submicron or nanoscale particles, such as LNPs, the mean HLB value should preferably be in the range of 3.5 to 5.5 (Log P value range of 1.22 to 2.04). The optimal mean HLB range or mean Log P range of all amphiphilic structures for the production of Dik-LNPs depends on the structure of the amphiphilic substances used. The weight ratio of the PEGylated components or substance (ii) to the hydrophobic amphiphilic substances also plays an important role. A preferable range here is, for example, approximately 2:1 to 1:10.

In addition to PEGylated substances such as ALC-0159, PEG2000-DMG, polysorbates, cetromacrogols and PEG stearates, poloxamers can also be used for the formulation of disk LNP used in the pharmaceutical industry.

Suitable hydrophobic amphiphilic substances used as carrier substances are phosphatidylcholines, ALC-0315, SM-102, liquid triglycerides, octyldodecanol, phytosterol, cholesterol, monoglycerides, diglycerides, sorbitan fatty acid esters.

It is striking and surprising that the disk structures can also be obtained above the submicron range, i.e. in the u range, which can be observed from the relatively narrow Gaussian particle size distribution.

Such disk structures can certainly also be considered as pharmaceutical carriers because the loading quantity of amphiphilic active ingredients is significantly greater here than in the submicron range.

The following Table 5 shows corresponding compositions. Comparable properties can be obtained in alternative compositions or preparations with phosphatidylcholine or a (further) active ingredient.

TABLE 5

| | | | | |
|---|---|---|---|---|
| 1st batch (Phase A) | | | | |
| Glycerol monolaurate | 6.4 | 4.5 | 5.2 | 4.1 |
| [wt. %] | | | | |
| PEG 40 stearate [wt. %] | 8.1 | 5.7 | 6.5 | 5.2 |
| Capric acid/caprylic | 43.1 | 33.1 | 38.2 | 37.6 |
| acid triglyceride [wt. %] | | | | |
| Phytosterol [wt. %] | 2.6 | 2.8 | 3.3 | 5.8 |
| 2nd batch (phase B) | | | | |
| Sucrose [wt. %] | 11.4 | 10.7 | 13.4 | 13.5 |
| Water [wt. %] | 28.4 | 43.1 | 33.4 | 33.7 |
| Total | 100.00 | 100.00 | 100.00 | 100.00 |
| Particle size [nm] | 460 ± 100 | 360 ± 90 | 530 ± 150 | 180 ± 80 |
| Mean value HLB | 4.17 | 3.93 | 3.96 | 3.47 |
| Mean value Log P | 1.66 | 1.81 | 1.76 | 2.05 |
| (approx.) | | | | |

In order to produce LNPs with a disk structure, the PEGylated surfactants with an HLB value calculated according to Griffin of at least 13 (Log P value at most 0.46) and amphiphilic non-water-soluble substances with an HLB of at most 7 (Log P value at least 0.93) or of at most 11 (Log P value at least 0.55) for the phospholipid should be used. The average HLB value from the amphiphilic and pegylated substances can preferably be in the range from 3.5 to 5.5 (Log P in the range from 1.22 to 2.05).

### Variation of the Water-Insoluble Amphiphilic Substances

In the submicron disk LNPs described here, the concentrations within the water-insoluble amphiphilic substances can be varied if the mean HLB value or Log P value remains within the favorable range described. In the example described here (Table 6), the concentration of phytosterol is specifically increased without leaving the desired particle size range. It should also be noted that phytosterols have an antioxidant effect. Comparable properties are obtained in alternative compositions or batches with phosphatidylcholine.

TABLE 6

| 1st batch (Phase A) | |
|---|---|
| Glycerol monolaurate [wt. %] | 5.5 |
| PEG 40 stearate [wt. %] | 8.7 |
| Capric acid/caprylic acid | 25.1 |
| triglyceride [wt. %] | |
| Phytosterol [wt. %] | 8.2 |
| Glycerol [wt. %] | 3.8 |
| 2nd batch (phase B) | |
| Sucrose [wt. %] | 9.6 |
| Water [wt. %] | 39.1 |
| Total | 100.00 |
| Particle size [nm] | 480 ± 130 |
| Mean value HLB | 4.8 |
| Mean log P | 1.43 |

Formulations with Water-Insoluble Amphiphilic Active Ingredients

As the above formulation using the example of the water-insoluble amphiphilic active ingredient shows, it is therefore also possible to incorporate such an active ingredient into a disk LNP formulation.

Other possible, in particular non-water-soluble amphiphilic active ingredients are, for example, mRNA, retinol, cortisone, antibiotics such as tetracyclines or benzoylpenicillins, diclofenac, estradiol, estradiol hemihydrate, salicylic acid, aspirin, indometacin, essential oils, amphiphilic UVA and UVB filters in a non-limiting manner. All water-soluble compounds that can interact with the carrier substances of the Disk-LNP, such as mRNA, can be considered as water-soluble active pharmaceutical ingredients. Disk-LNPs containing active pharmaceutical ingredients can be applied intramuscularly, intravenously, orally or topically. In topical application, Disk-LNPs are characterized by good and controlled penetration behavior. The penetration behavior of the LNPs described here can be further controlled by using panthenol.

Table 7 shows a further composition and related batches. Finishing with active ingredients leads to comparable properties.

TABLE 7

| | |
|---|---|
| 1st batch (phase A) | |
| Polysorbate 80 [wt. %] | 14 |
| Tocopherol [wt. %] | 12 |
| Phospatidylcholine [wt. %] | 1.6 |
| Phytosterol [wt. %] | 2.5 |
| Capric acid/caprylic acid | 41.9 |
| triglyceride [wt. %] | |
| 2nd batch (phase B) | |
| Saccherose [wt. %] | 4.5 |
| Water [wt. %] | 23.5 |
| | 100 |
| Particle size [nm] | 380 ± 80 |
| mean HLB or Log P | 4.2 or 1.66 |

### Preparation Process for Disk LNP

The following methods are described for producing disk LNP. In particular, a direct preparation process or an inverse method can be used.

In the inverse method, the entire carrier phase based on the PEGylated surfactant and the non-water-soluble substances is presented in its liquid phase formed by melting and the water phase is added in portions.

Surprisingly, a hot-cold process can be used for producing disk LNP using the inverse method. Although the phase A described in the previous examples is heated to 80° C. to 90° C., the water phase (phase B) with a temperature of 25° C. to 30° C. can either be added slowly to this phase or stirred in in portions. A temperature of the water phase B of more than 40° C. during stirring, on the other hand, does not lead to optimum results.

For the application of the inverse method, it is advantageous to add an aqueous phase B comprising a polyol content of preferably 20 wt. % to 50 wt. % to the molten phase A, which comprises a temperature of 50° C. to 65° C., in particular at a temperature of 25° C. to 30° C., with stirring, and then to incorporate phase C with stirring.

Please also refer to Table 8 for the relevant batches:

TABLE 8

| | |
|---|---|
| 1st batch (Phase A) | |
| Glycerol monolaurate [wt. %] | 5.5 |
| PEG 40 stearate [wt. %] | 8.7 |
| Capric acid/caprylic acid | 25.1 |
| triglyceride [wt. %] | |
| Phytosterol [wt. %] | 8.2 |
| Glycerol [wt. %] | 3.8 |
| 2nd batch (phase B) | |
| Saccherose [wt. %] | 9.6 |
| Water [wt. %] | 28.8 |
| Further batch (Phase C) | |
| Saccherose [wt. %] | 10.3 |
| | 100 |
| Particle size [nm] | 480 ± 130 |
| mean HLB or mean log P | 4.8 or 1.43 |

When directly incorporating (Table 9) phase A, which comprises a temperature of 80° C. to 90° C. and represents a homogeneous liquid phase, into the aqueous phase B, which preferably contains 15 wt. % to 25 wt. % of the PEGylated substance, the following procedure is expedient for obtaining a liquid-crystalline disk phase.

Phase A is added slowly or in portions while stirring to phase B, which preferably has a temperature of 25° C. to 40° C. During the addition of the phase, the viscosity of the mixture obtained increases with increasing concentration and reaches a highly viscoelastic gel range at the end of the addition. The gel phase obtained is cooled to 20° C.

TABLE 9

| 1st batch (Phase A) | |
|---|---|
| Glycerol monolaurate [wt. %] | 5.7 |
| PEG 40 stearate [wt. %] | 6.8 |
| Capric acid/caprylic acid | 26 |
| triglyceride [wt. %] | |
| Phytosterol [wt. %] | 8.5 |
| Glycerol [wt. %] | 3.9 |
| 2nd batch (phase B) | |
| Saccherose [wt. %] | 13.3 |
| Water [wt. %] | 33.5 |
| Saccherose [wt. %] | 2.2 |
| | 100 |
| Particle size [nm] | 640 ± 170 |

The methods, in particular the direct method, can also be used with a cascaded continuous method. Both methods are suitable for producing lamellar liquid crystalline as well as cubic liquid crystalline structures, which show stable LNP structures when diluted. Comparable properties can be obtained in alternative compositions or batches with phosphatidylcholine or a (further) active ingredient.

When stirring the gel phase, it may be the case that the stirring energy dissipates into heat. This can be counteracted by cooling.

### Electrolyte Compatibility

In addition to the polyols, the water phase can also contain electrolytes, which can be used to adjust the pH of the system, for example. The electrolyte concentrations used for this purpose have no significant negative influence on the formed structure of the lyotropic liquid-crystalline phase. To set the pH value using an aqueous electrolyte solution, the water concentration should be selected so that it does not leave the liquid-crystalline range so that all areas of the individual compartments can be reached.

Producing LNP with PEGylated Surfactants and Phosphatidylcholine

A most favorable packing parameter for the production of lamellar liquid-crystalline structures with a spherical structure is in particular in the range Pₖᵣ=0.5, which corresponds to an HLB value range of 10 to 12 (in particular Log P in the range of 0.50 to 0.62). In particular, vesicle-like structures can be formed in this HLB range, which show a cubic shape. If the cubic form is diluted with water, spherical LNPs are formed.

If a PEGylated surfactant is used for the production of these phases, it is evenly distributed over the entire lamella of the vesicle. However, vesicles formed with PEGylated substances are less suitable as carriers for active substances that are to be introduced into a human cell, as the PEG residues of the pegylated substance can form a steric barrier with the cell.

The load of water-insoluble amphiphilic substances, such as phosphatidylcholine, cholesterol or phytosterols, is also sometimes very limited. Phytosterols have the advantage over cholesterol that they have been shown to have antioxidant properties, which represents protection against the oxidation of sensitive active substances.

Further formulations according to Table 10 are given below, also to demonstrate the above configuration. The table also shows the results of the formulations of liquid-crystalline gel structures.

TABLE 10

| 1st batch (phase A) | | | | |
|---|---|---|---|---|
| Phospatidylcholine [wt. %] | 4.5 | 4.4 | 4.4 | 4.5 |
| Capric acid/caprylic acid | 5.4 | 9.1 | 16.8 | 16.6 |
| triglyceride [wt. %] | | | | |
| PEG 40 stearate [wt. %] | 19.1 | 16.8 | 9.1 | 10.1 |
| Phytosterol [wt. %] | 0.9 | 3.5 | 3.5 | 4.1 |
| Glycerol [wt. %] | 26.6 | 25.6 | 25.7 | 23.8 |
| 2nd batch (phase B) | | | | |
| Water [wt. %] | 43.5 | 40.6 | 40.6 | 41.0 |
| | 100.00 | 100.00 | 100.00 | 100.00 |
| Particle size [nm] | 400 ± 100 | 320 ± 70 | 6.000 | 8.000 |
| Mean value HLB | 11.7 | 10.1 | 9.5 | 9.1 |
| Mean value Log P | 0.52 | 0.61 | 0.65 | 0.69 |

The liquid crystalline gel structures with an average HLB value of 11.7 (Log P=0.52) and 10.1 (Log P=0.61) represent clear gels that can be assigned to the cubic structure. These are spherical or ellipsoidal vesicle structures.

The gel structures of the formulations with an average HLB value of 9.5 and 9.1 (Log P=0.65 and 0.69)) show a milky structure and thus cannot be assigned to the cubic form. It can be assumed that at least two structures overlap in the gel phase. The wide scattering of the particle size, which is in the scaled μm range, shows that the gel comprises a non-uniform liquid crystalline structure and therefore does not form a homogeneous system. Such an undesirable effect is also sometimes seen with the mRNA vaccines currently in use.

A Horiba la-920 Particle Size Analyzer was used to measure the particle size.

Producing the liquid crystalline gels effects the following procedure. The components contained in phase A, phosphatidylcholine, which is derived from soy lecithin, PEG 40 stearate and glycerol, are heated to 70° C. and stirred until a homogeneous clear solution is formed. In parallel, phytosterol is dissolved in capric acid/caprylic acid triglyceride at 80° C. with stirring and then stirred homogeneously into the solution consisting of phosphatidylcholine, PEG 40 stearate and glycerol. To this phase A, phase B water comprising a temperature of 20° C. to 30° C. is incorporated in portions while stirring with laminar flow.

A substructure of the vesicle-like liquid crystalline lamellar structure is the disk structure, which is formed by a surfactant with an HLB value calculated according to Griffin of at least 13 (in particular Log P of at most 0.46) and non-water-soluble amphiphilic substances with an HLB of in particular at most 6 (in particular Log P of at least 1.11). The HLB value averaged from the HLB values of the surfactants with HLB of at least 13 and the non-water-soluble amphiphilic substances with HLB of at most 6 is here preferably an HLB of at most 5 (in particular Log P of at least 1.36).

Liquid-crystalline disk structures have a cylindrical geometry (see e.g. FIG. 1A). In particular, in this geometry, the outer surface is primarily, in particular almost exclusively, covered with pegylated surfactants in accordance with the law of packing parameters, while the circular base surfaces are mainly covered with the non-water-soluble amphiphilic components (see e.g. FIG. 1B).

TABLE 11

| 1st batch (phase A) | | | |
|---|---|---|---|
| Phospatidylcholine [wt. %] | 5.9 | 6.8 | 7.7 |
| Capric acid/caprylic acid | 14.0 | 16.4 | 18.1 |
| triglyceride [wt. %] | | | |
| PEG 40 stearate [wt. %] | 5.0 | 6.8 | 8.6 |
| Phytosterol [wt. %] | 3.1 | 3.7 | 4.1 |
| Glycerol [wt. %] | 27.1 | 29.6 | 30.1 |
| 2nd batch (phase B) | | | |
| Water [wt. %] | 44.9 | 36.6 | 31.4 |
| | 100.00 | 100.00 | 100.00 |
| mean HLB or Log P | 4.8 or 1.43 | 5.1 or 1.33 | 5.4 or 1.25 |
| Particle size [nm] | 1.200 ± 300 | 540 ± 150 | 400 ± 90 |

Surprisingly, the same component matrix that was used for producing liquid crystalline spherical vesicle structures can also be used to produce liquid crystalline disk LNP structures (Table 11).

Producing the disk structures shown here effects the same procedure as producing the vesicle structures shown above. The producing of disk structures can be effected after both the direct and inverse phase. To produce a disk structure, PEGylated surfactants with an HLB value calculated according to Griffin of at least 13 (in particular Log P of at most 0.46) and amphiphilic non-water-soluble substances with an HLB of in particular at most 5 (in particular Log P of at least 1.36) should be used. The average HLB value from the amphiphilic and pegylated substances is preferably in the range from 4.5 to 6 (Log P in the range from 1.11 to 1.54) for the combination of PEGylated substances and phosphatidylcholine.

The PEGylated surfactant used in the present formulations is PEG 40 stearate. If necessary, free PEG is used, which is known to contain at least 20% to 30% free PEG. This means that the effective use concentration of PEG 40 stearate in the present formulations must be set approximately 30 percent lower. This may possibly contain free PEG (in particular about 20% to 30%), which is known to the person skilled in the art. In this case, the effective use concentration or amount of PEG 40 stearate or the corresponding PEGylated surfactant in the present formulations must be set lower by the corresponding percentage value.

The formulations described here are stable over a wide temperature range from around −15° C. to 40° C. The particle size changes only insignificantly at best over a storage period of 6 weeks.

Comparable properties can be obtained in alternative compositions with a (further) active ingredient.

Producing Spherical Lamellar Vesicle Structures as Carriers for mRNA Systems

The already approved mRNA vaccines against SARS-CoV-2 contain the PEGylated surfactants and water-insoluble amphiphilic substances described below, which can also be assigned an HLB value. The carrier substances ALC-0159, DMG-PEG 2000, ALC-0315 and SM-102 are combined with DSPC and cholesterol. If a homogeneous and coherent liquid-crystalline vesicle structure is to be formed from these substances, the average HLB value formed from all the substances involved should preferably be in the range HLB of 9.8 to 12. The following percentage composition of the components should serve as an example of a suitable composition of amphiphilic components, in particular with reference to the above configurations.

TABLE 12

| | |
|---|---|
| ALC-0159 [wt. %] | 67.7 |
| ALC-0315 [wt. %] | 20.8 |
| DSPC [wt. %] | 7.8 |
| Cholesterol [wt. %] | 3.6 |
| Total | 100.0 |

Such a composition will produce lamellar spherical vesicle compartments in a liquid-crystalline structure produced therefrom, which transition into a nano-scale stable structure when diluted with an aqueous solution. Aqueous mRNA solutions can be stirred into the liquid-crystalline structure easily and homogeneously distributed.

Producing Disk Lamellae as Carriers for mRNA Systems

For the formation of disk lamellae or disk LNPs, a preferably average HLB value formed from the pegylated and water-insoluble amphiphilic substances of HLB=4.5 to 6 or Log P=1.11 to 1.54 is advantageous.

For example, the following composition can be used:

TABLE 13

| | |
|---|---|
| ALC-0159 [wt. %] | 17.1 |
| ALC-0315 [wt. %] | 57.1 |
| DSPC [wt. %] | 11.4 |
| Cholesterol [wt. %] | 14.3 |
| Total | 100.0 |

Such a composition will produce lamellar disk compartments or disk LNPs in a liquid crystalline structure produced therefrom, which transition into a nano-scale stable structure when diluted with an aqueous solution. Aqueous mRNA solutions can be stirred into the liquid crystalline structure easily and homogeneously distributed.

Advantages of Submicron or Nanoscale Disk LNP for the Incorporation of mRNA

The small disk LNPs comprise a very large specific surface area, which allows mRNA to be incorporated, in particular at low temperatures, in such a way that it can interact electrostatically with cationic carrier components in particular and thus be distributed very evenly and selectively in the disk compartments under laminar stirring.

Since the lyotropic liquid-crystalline phase shows very low water activity, the risk of oxidation phenomena or other chemical reactions is also greatly suppressed and mRNA is very well protected even at temperatures above the freezing point of water.

The low water activity of the liquid-crystalline phase is due on the one hand to the binding capacity of the water to the hydrophilic head groups of the PEGylated surfactants and the water-insoluble amphiphilic components and on the other hand to the water-binding capacity of the polyols used.

The particle size is formed below the phase transition temperature by intensive stirring in the laminar flow range. The temperature range of the phase transition is in the range of the temperature at which the crystallization of substances from the liquid homogeneous molten phase of the PEGylated and amphiphilic substances begins. The stirring time in this range determines the particle size. The longer intensive stirring is carried out, taking into account the laminar flow, the smaller the particles become.

The critical phase transition temperature can be determined using DSC measurements. The DSC measurements also show the transition area of the liquid form of the lamellar disk structure at the phase transition temperature into a solid gel structure.

Producing with Ingredients Phosphatidylcholine

For producing liquid-crystalline LNP disk structures, the first step should be to heat the entire phase of all PEGylated and amphiphilic substances until a homogeneous clear to slightly transparent mixture appears.

If the formulation contains phosphatidylcholines and cholesterol or phytosterol, the following procedure is advantageous. First, the PEGylated surfactant is melted at 70° C. to 80° C. together with glycerol or another preferably liquid polyol and a phosphatidylcholine. The polyol is supplied into this phase to dissolve poorly soluble phosphatidylcholines, such as distearoylphosphatidylcholine. Phosphatidylcholines are very well described with regard to the phase transition temperature. To dissolve a phosphatidylcholine in a liquid polyol, the temperature should be above the phase transition temperature.

The polyol content, based on the lipid phase, which in particular is composed of amphiphilic water-insoluble substances, should preferably be 5 wt. % to 50 wt. %.

The remaining amphiphilic substances are then added and stirred homogeneously. Finally, the cholesterol or phytosterol is added and stirred until a homogeneous liquid is formed. The temperature should now preferably be 80° C. to 90° C.

Surprisingly, the water phase, which preferably consists of a polyol solution, can now be added slowly or stepwise with stirring at a temperature of preferably 20° C. to 50° C., at a concentration until a water-soluble gel phase has been set.

Transferring the Results to mRNA Vaccines

Glycerol monolaurate is chosen because with an HLB of 5.2 (Log P of 1.30) it corresponds approximately to that of distearoylphosphatidylcholine HLB=4.5 (Log P=1.54). It could be shown that approximately the same HLB values or Log P values in the formulation of Disk-LNP lead to the same structural results, in order to achieve a range for Disk-LNP with the raw materials used in mRNA vaccines, the formulation must be designed in particular in such a way that the concentration of the pegylated substance together with the water-insoluble components results in an average HLB value of HLB=4.5-6.0 (Log P=1.11-1.54).

In the production of mRNA Disk-LNP, an aqueous mRNA solution is added after liquid crystalline gel formation is complete. The final pH value is then set with another aqueous phase.

### Producing Disk LNP for Topical Applications

Disk-LNPs are very suitable for topical applications, in particular if they contain phosphatidylcholines in their matrix, which ensure good bioavailability, as they guarantee good penetration of the active ingredient after application due to good film formation on the skin and also feel pleasant on the skin during and after application. If disk LNPs also contain phytosterols as amphiphilic substances that have an antioxidant effect, for example, they are also suitable in particular for active ingredients that tend to oxidize in the air, such as tocopherols or retinol. In this context, oxidation protection can also be provided through the use of vitamin C, in particular together with panthenol. The use of vitamin D, in particular as a lamellar or membrane antioxidant, also leads to oxidation protection.

For example, concentrates or the like can be incorporated into disk lamellae in this context.

### Advantages of the Disk LNP According to the Invention

A surprising result of the disk LNP described in the present case is the production provided for this purpose according to the invention or the method relating thereto, which is also accompanied by a simplification of the producing of the particles. In particular, existing or conventional production facilities in the pharmaceutical industry can also be used. Larger batches can also be produced easily with conventional mixing systems. Another feature is the consistent quality of the products obtained. Formulations can also be quickly adapted to other active ingredients without major research effort.

### Online Production Control

For production monitoring, the following characteristic parameters for each system can also be checked online, for example. Characteristic parameters are, for example, temperature, viscosity and conductivity. By measuring these characteristic parameters online, it is possible to carry out the production of disk LNP and other liquid-crystalline LNP in a computer-controlled manner, for example. Computer-controlled and monitored producing is also advantageous for production in most regions worldwide in order to get pandemics under control more quickly.

### Summary

Producing and formulating lytropic liquid crystalline vesicle and disk LNPs or compositions thereof, which
comprise phosphatidylcholine, in particular in combination with phytosterol or cholesterol,
whose mean HLB value of the pegylated substances used is HLB≥13 and of the water-insoluble amphiphilic substances in particular is HLB≤6 (the same applies to the correlating log P values),
can be produced using the hot-cold method,
comprise good storage stability over a wide temperature range,
in particular comprise a water activity of less than 1,
the producing of which can be effected in just a few steps,
can be produced under laminar flow conditions, in particular with stirring.
allow the most important process parameters to be monitored during producing and the method to be controlled using IT technology,
can be produced in a direct or inverse preparation process,
can be produced in such a way that coherent liquid crystalline phases are made possible or improved by the addition of polyols,
make it possible to homogeneously distribute water-soluble active substances such as mRNA at low temperatures by stirring,
enable the stable incorporation of amphiphilic active ingredients specifically into the LNP matrix,
show a narrow Gaussian particle size when diluted with water, in the pharmaceutical field can be applied intramuscularly, intravenously, orally and topically.

### Part D:

### Further Explanations on the Objective

Producing LNPs, in particular mRNA LNPs, is difficult and sometimes leads to very inhomogeneous systems in the state of the art, which also requires a very complex application during application. Known systems must be cooled to low temperatures for storage. In addition, complex handling is required before administration (unconditional avoidance of shear forces that would occur during shaking, complex dilution, vertical tilting before dilution, short storage time after thawing, use of special cannulas, no prolonged transport after dilution, etc.). In addition, particles in the high μm range are often present

The reasons for the inhomogeneity of the vaccine can be attributed to several factors. In particular, sufficient solubility of the main components phosphatidylcholine and cholesterol has been problematic to date. In existing methods, phosphatidylcholines are dissolved at first in ethanol, which then has to be evaporated in a next step using a vacuum. The solubility of cholesterol is usually achieved using chloroform, which also has to be removed from the system using a vacuum. With the previous preparation processes, there can also be considerable batch-to-batch fluctuations in quality.

Often the particles, in particular vesicles containing phosphatidylcholines together with cholesterol, are produced by ultrasonic methods, the input of energy of such methods is not insignificant. For example, cavitation bubbles imploding in the sonication field can cause intense shock waves in the liquid and liquid jets of very high velocity. These liquid jets reduce the size of the individual droplets in the sonicated liquid and “mix” them. Such a method therefore often leads to a very high local input of energy and thus represents a method that cannot be easily controlled overall. The input of energy of ultrasonic processors for the laboratory is 50 W to 400 W, while 500 watt to 16 KW processors are used for industrial production. Overall, common methods for producing LNPs, which serve as carriers for mRNA, are described in the specialist literature as very complicated and costly. The reason for the complex preparation process is the very poor water solubility of DSCP and cholesterol.

### Further Explanations for Problem Solving

This problem can be solved in particular by dissolving DSCP in an aqueous polyol solution. Suitable polyols are, for example, propylene glycol, glycerol or sucrose. Surprisingly, most phosphatidylcholines can be completely dissolved at room temperature in a sucrose-water mixture or in other aqueous polyol mixtures.

Soy lecithin also contains glycolipids. Glycolipids are phosphorus-free structural lipids or membrane lipids (components of cell membranes) in which one or more monosaccharides or oligosaccharides are glycosidically bound to a lipid molecule. Glycolipids are found in all tissues, but only on the outside of the lipid bilayer. The soy lecithin used below is a product enriched to 45 percent phosphatidylcholine (Lipoid® S45). The proportion of glycolipids can be assumed to be around 5 percent. In lecithin gels based on aqueous polyol solutions, water-soluble surfactants with an HLB value of at least 13 can also be incorporated according to the invention, in particular cold. Since the surfactants ALC-0159 and DMG-PEG 2000 used in the mRNA vaccines have a sufficiently high HLB value, these can also be cold incorporated into lecithin gels. Relatively high branched-chain alcohols comprising an HLB of 2 to 4, such as octyldodecanol, ALC-0315 or SM-102, can also be incorporated into such lecithin surfactant gels under lamellar stirring flow at room temperature or lower temperatures. According to the invention, stable gel-like semi-transparent structures are formed which can be diluted with water and comprise a submicron or nanoscale particle size when water is added slowly. In such gels, mRNA can also be incorporated at low temperatures, e.g. below room temperature.

The following table shows a corresponding recipe:

TABLE 14

| | |
|---|---|
| Soy lecithin [wt. %] | 13.0 |
| Sugar [wt. %] | 27.9 |
| Water [wt. %] | 35.7 |
| Octyldodecanol [wt. %] | 20.5 |
| Polysorbate 80 [wt. %] | 2.9 |
| Total | 100.0 |

The structure resulting from this formulation is a semi-transparent gel that forms submicron or nanoscale particles when diluted with water. Phytosterol or cholesterol can also be dissolved in the surfactant used. Other lipophilic active ingredients, in particular with an HLB value in the range of 1 to 5, such as retinol, tocopherol, tetracyclines etc. can also be incorporated into this system. Hydrocortisone with an HLB=4.5 is poorly soluble in water but very soluble in polyols. It can be assumed that substances with an HLB value HLB=4-5 have the same solubility spectrum as cortisone. The same applies when using the correlating Log P values.

### Producing LNPs

To produce another stable LNP system, it is preferably that the components are divided into two phases. The preferably division of the components depends on the solubility of the selected phosphatidylcholine in an aqueous polyol mixture. In the present case, the energy introduced to comminute the particles is in particular 1 to 2 watts/kg of emulsion. If the selected phosphatidylcholine is soluble quickly enough in an aqueous polyol mixture, for example in an aqueous sugar solution, this solution forms a phase. The second phase consists of a PEGylated surfactant, a branched-chain alcohol and a cholesterol or phytostearin. Preferably, the PEGylated surfactant should comprise an HLB value of more than 13, the branched-chain alcohol an HLB value in particular in the range from 2.5 to 3.5 and the cholesterol or phytostearin an HLB=0.9 to 1.1. The same applies when using the correlating Log P values. In the present example, the phosphatidylcholine phase remains at about 20° C. The second phase is heated to 70° C. to 80° C. and stirred until the cholesterol is completely dissolved. The phase is then cooled to 45° V to 50° C. and slowly added to the phosphatidylcholine phase while stirring to ensure lamellar flow behavior. This creates a non-flowable liquid-crystalline gel. The resulting gel can be diluted with water while stirring under laminar flow. Depending on the stirring time and stirring speed, the disk particles produced have a particle size of 170 nm to 250 nm.

A different division of the phases results from the natural solubility of the PEGylated component in water and aqueous polyol systems due to the HLB value or Log P value. Before adding the second phase, this phase described here should comprise a temperature of 25° C. to 45° C. The second phase then consists of the phosphatidylcholine and cholesterol, which are dissolved in the branched-chain alcohol at approx. 70° C. to 80° C. Before being added to the first phase, the solution is preferably cooled to a temperature of <70° C.

In phase partitioning, it is also possible that part of the phosphatidylcholine and/or cholesterol is present in both the first and second phase.

All substances used in this trial are listed in the European Pharmacopoeia.

TABLE 15

| | |
|---|---|
| Soy lecithin [wt. %] | 3.5 |
| Sugar [wt. %] | 16.3 |
| Water [wt. %] | 51.2 |
| Macrogol-40-stearate [wt. %] | 2.9 |
| Octyldodecanol [wt. %] | 17.8 |
| Phytosterol [wt. %] | 8.3 |
| Total | 100.0 |

A substitution of the phytosterol with cholesterol leads to the same results. The PEGylated surfactant macrogol-40 stearate is a PEG-40 stearate with an HLB value=16.9 and is very similar to ALC-0159. The branched-chain fatty alcohol octyldodecanol has an HLB=3 and is therefore very similar to ALC-0315 in its physical properties. ALC-0315 is a branched-chain amphiphilic cationic synthetic lipid with a terminal OH group.

The disk structures or disk LNPs are geometrically based on a cylinder structure. The shell of a disk cylinder is mainly covered with hydrophilic surfactants, which in particular comprise a packing parameter of 0.3 to 0.5, which corresponds to an HLB value of the substances of 10 to 17 (in particular Log P of about 0.34 to 0.61). The base area is occupied by amphiphilic substances with a packing parameter of about 1. From the ratio of the hydrophilic and lipophilic amphiphilic components, the lamellar occupancy and the corresponding diameters of the LNPs can be determined or controlled.

### Continuous Production

Reference can also be made to FIGS. 6A and 6B. The residence time depends on the flow rate and vessel size. If vessels with a volume of 300 ml are used in the stirring cascade, the average residence time can be around 10 to 15 seconds per stirring stage. In this case, the stirring chambers are filled with aliquots from the respective storage devices or storage chambers according to a2 to d2 and passed from stirring chamber A2 (FIG. 6A) to the final stage D4 (FIG. 6A). The end product can be removed from stirring stage D2. If the subsequent quality control meets the specifications, the individual stirring stages for subsequent batch production no longer need to be purified. The method can also be carried out semi-continuously, in particular to enable batch quality control. The present producing process can be carried out with very little residence time in the respective stirring chamber.

The method ensures an extremely low temperature load on the active ingredient to be encapsulated, such as mRNA. Corresponding reference can also be made to FIG. 6B.

### Part E: Agitator for Forming a Laminar Flow

### Spiral Stirrer

The spiral agitator is suitable for homogenizing highly viscous media. This stirring device comprises a ribbon-shaped spiral, which is held on the shaft with webs. In contrast to the propeller, in this case the axial flow is not caused by pressure differences but by displacement effects in the laminar flow area. With a diameter ratio of 0.9 to 0.99, the spiral agitator is a wall-mounted agitator.

### Wire Stirrer

Wire agitators are also particularly suitable for homogenizing highly viscous media. The formation of a turbulent flow can be counteracted by adjusting the wire arrangement and wire thickness.

### Ekato-Paravisc®-Stirring Equipment

This agitator belongs to the group of wall-mounted agitators. These agitators work in particular in the laminar range, and circulation of the container contents is achieved by forced conveying. Axial mixing is achieved by the special shape and arrangement of the two main blades. Depending on the application, this agitator can be operated with both downward and upward conveying.

### Part E: Vitamin C as an Active Ingredient Model Substance

Based on the formulation listed in Table 16, a further composition or a corresponding carrier system is produced, wherein vitamin C is used as a model substance for mRNA active ingredients.

TABLE 16

| | |
|---|---|
| 1st batch (Phase A) | |
| Panthenol [wt. %] | 20.3 |
| Water [wt. %] | 5.9 |
| Vitamin C [wt. %] | 16.9 |
| 2nd batch (phase B) | |
| Phosphatidylcholine [wt. %] | 6.0 |
| PEG 40 stearate [wt. %] | 11.3 |
| Glycerol monolaurate [wt. %] | 2.2 |
| Capric acid/caprylic acid | 23.0 |
| triglyceride [wt. %] | |
| 3rd batch (phase C) | |
| Water [wt. %] | 14.4 |
| Total | 100 |
| Particle size [nm] | 140 ± 0.03 |
| mean HLB or Log P | 6.1 or 1.08 |

Solid vitamin C is relatively stable, but normally decomposes relatively quickly in aqueous solution, in particular due to oxidation processes. In contrast, the vitamin C in the present composition or carrier system comprises a high stability, even with regard to the relatively high amount of vitamin C used of more than 15 wt. %. The high solubility in panthenol, which even exceeds the solubility in water, is also surprising. In other polyols, such as glycerol and propylene glycol, the solubility is lower. Without wanting to limit or refer to this theory, the high solubility of vitamin C in panthenol can be explained by its cationic behavior. If a high stability is achieved even for high amounts of vitamin C of more than 15 wt. %, in particular in combination with panthenol or other polyols, then this is a clear indication that mRNA active ingredients can also be stabilized on this basis. Overall, the excellent effect using vitamin C as a model substance for mRNA active ingredients shows that mRNA active ingredients can be stabilized accordingly.

Overall, the embodiments show the advantages and special features associated with the present invention.

1-171. (canceled)

172. A method for producing a lipid-based carrier system in the form of lipid nanoparticles, which carrier system is appropriate to be provided with at least one pharmaceutical active ingredient,
wherein the method comprises the following steps:
the carrier system is formed by several components, wherein the components of the carrier system are brought into contact and into interaction with each other in such a way that an agglomeration of the components is effected, so as to form the carrier system in the form of lipid nanoparticles,
wherein the components of the carrier system comprise the following substances:
(i) at least one phospholipid,
wherein the phospholipid fulfills at least one of the following provisos: the phospholipid comprises an HLB value according to Griffin of at most 11 and the phospholipid comprises a Log P value of at least 0.55;
(ii) at least one of a non-ionic amphiphilic and surfactant substance selected from the group consisting of polyglycerol esters, alkyl polyglycosides, amphiphilic and surfactant PEGylated substances and combinations thereof,
wherein the at least one of a non-ionic amphiphilic and surfactant substance fulfills at least one of the following provisos: the at least one of a non-ionic amphiphilic and surfactant substance comprises an HLB value according to Griffin of at least 13 and the at least one of a non-ionic amphiphilic and surfactant substance comprises a Log P value of at most 0.46;
(iii) optionally at least one of a steroid and a secosteroid,
wherein the at least one of a steroid and secosteroid fulfills at least one of the following provisos: the at least one of a steroid and secosteroid comprises an HLB value according to Griffin of at most 1.5 and the at least one of a steroid and secosteroid comprises comprises a Log P value of at least 5.40;
(iv) optionally at least one cationic lipid,
wherein the cationic lipid fulfills at least one of the following provisos: the cationic lipid comprises an HLB value according to Griffin of at most 4 and the cationic lipid comprises a Log P value of at least 1.76;
(v) optionally at least one mono-, di-, tri- or polycarboxylic acid ester of a polyol,
wherein the mono-, di- or tricarboxylic acid ester of glycerol fulfills at least one of the following provisos: the mono-, di- or tricarboxylic acid ester of glycerol comprises an HLB value according to Griffin of at most 8 and the mono-, di- or tricarboxylic acid ester of glycerol comprises a Log P value of at least 0.80;
wherein the method is carried out in a liquid medium, which medium is liquid at a temperature in the range from 20° C. to 40° C. at ambient pressure and which medium is in the form of a dispersion medium, such that, after formation of the carrier system, a dispersion of the carrier system in the liquid medium results;
wherein agglomeration of the components forming the carrier system is carried out by introducing energy into the liquid medium comprising at least part of the components forming the carrier system, wherein the energy introduced is set in such a way that an at least substantially laminar flow is effected in the liquid medium comprising at least part of the components forming the carrier system;
wherein a continuous method control is carried out for monitoring at least one of the method status and the method progress comprising monitoring one or more method parameters selected from the group consisting of input of energy, input and removal of thermal energy, flow behavior, mass throughput, volume flows or combinations thereof; wherein method control is carried out by monitoring at least one process parameter selected from the group consisting of temperature, conductivity, viscosity and pH value of the liquid medium comprising the components forming the carrier system;
wherein the method is carried out at least substantially in the absence of ethanol.

173. The method according to claim 172,
wherein the amount of energy introduced is set in such a way that a Reynolds number Rₑ of at most 2,000 is effected.

174. The method according to claim 172,
wherein the method is performed in the presence of at least one active ingredient, wherein the at least one active ingredient is a component of the carrier system and wherein the carrier system comprises the at least one active ingredient.

175. The method according to claim 173,
wherein the active ingredient is characterized by at least one of the following features:
the active ingredient is selected among therapeutic and prophylactic active ingredients and therapeutic and prophylactic pharmaceutical active ingredients and combinations thereof;
the active ingredient is selected from the group consisting of at least substantially water-insoluble active ingredients, at least substantially water-insoluble amphiphilic active ingredients, water-soluble active ingredients and combinations thereof;
the active ingredient is selected from the group consisting of vaccine active ingredients, antibiotic active ingredients, antifungal active ingredients, antiviral active ingredients, antiphlogistic active ingredients, antipyretic active ingredients, antiallergic active ingredients, anticancer active ingredients, hormone active ingredients, anticoagulant active ingredients, vitamins, minerals, UV-absorbing substances and combinations thereof;
the active ingredient is selected from the group consisting of RNA, retinol, tocopherol, corticosteroids, antibiotics, diclofenac, estradiol, estradiol hemihydrate, salicylic acid, acetylsalicylic acid, indomethacin, essential oils, amphiphilic UVA filters and UVB filters, nicotinamide, and combinations thereof;
the active ingredient is selected from the group consisting of vaccine active ingredients, nucleic acid-based vaccine active ingredients, gene-based vaccine active ingredients, vaccine active ingredients encoding an antigen of a pathogen or a pathogen, and combinations thereof;
the active ingredient is selected from the group consisting of vaccine active ingredients, wherein the vaccine active ingredients are selected from the group consisting of DNA, RNA, messenger RNA, viral vectors, plasmids, plasmid DNA and combinations thereof;
the active ingredient is selected from the group consisting of mRNA-based vaccine active ingredients and messenger RNA, and combinations thereof;
the active ingredient is selected from the group consisting of mRNA-based vaccine active ingredients and messenger RNA, and combinations thereof, wherein the mRNA encodes a protein of a coronavirus;
the active ingredient is selected from the group consisting of proteins and antigens and combinations thereof.

176. The method according to claim 172,
wherein the method is performed in the presence of at least one pharmaceutical active ingredient, wherein the at least one pharmaceutical active ingredient is a component of the carrier system and wherein the carrier system comprises the at least one pharmaceutical active ingredient.

177. A lipid-based carrier system in the form of lipid nanoparticles,
wherein the lipid-based carrier system is obtainable by a method according to claim 172.

178. The lipid-based carrier system according to claim 177,
wherein the lipid-based carrier system further comprises at least one active ingredient.

179. The lipid-based carrier system according to claim 177,
wherein the lipid-based carrier system further comprises at least one pharmaceutical active ingredient.

180. The lipid-based carrier system according to claim 177,
wherein the lipid-based carrier system is in the form of lipid nanoparticles and comprises at least one active ingredient,
wherein the lipid-based carrier system is formed by several components, wherein the components of the carrier system have been agglomerated to form the carrier system,
wherein the components of the carrier system comprise the following substances:
(i) at least one phospholipid,
wherein the phospholipid fulfills at least one of the following provisos: the phospholipid comprises an HLB value according to Griffin of at most 11 and the phospholipid comprises a Log P value of at least 0.55;
(ii) at least one of a non-ionic amphiphilic and surfactant substance selected from the group consisting of polyglycerol esters, alkyl polyglycosides, amphiphilic and surfactant PEGylated substances and combinations thereof,
wherein the at least one of a non-ionic amphiphilic and surfactant substance fulfills at least one of the following provisos: the at least one of a non-ionic amphiphilic and surfactant substance comprises an HLB value according to Griffin of at least 13 and the at least one of a non-ionic amphiphilic and surfactant substance comprises a Log P value of at most 0.46;
(iii) optionally at least one of a steroid and a secosteroid,
wherein the at least one of a steroid and secosteroid fulfills at least one of the following provisos: the at least one of a steroid and secosteroid comprises an HLB value according to Griffin of at most 1.5 and the at least one of a steroid and secosteroid comprises comprises a Log P value of at least 5.40;
(iv) optionally at least one cationic lipid,
wherein the cationic lipid fulfills at least one of the following provisos: the cationic lipid comprises an HLB value according to Griffin of at most 4 and the cationic lipid comprises a Log P value of at least 1.76;
(v) optionally at least one mono-, di-, tri- or polycarboxylic acid ester of a polyol,
wherein the mono-, di- or tricarboxylic acid ester of glycerol fulfills at least one of the following provisos: the mono-, di- or tricarboxylic acid ester of glycerol comprises an HLB value according to Griffin of at most 8 and the mono-, di- or tricarboxylic acid ester of glycerol comprises a Log P value of at least 0.80;
wherein the carrier system is contained in a liquid medium in the form of a dispersion medium, which medium is liquid at a temperature in the range from 20° C. to 40° C. at ambient pressure;
wherein the carrier system together with the liquid medium forms a disperse composition comprising the carrier system and the liquid medium as the dispersion medium;
wherein agglomeration of the components forming the carrier system has been effected by introducing energy into the liquid medium comprising at least part of the components forming the carrier system, wherein the energy introduced has been set in such a way that an at least substantially laminar flow has been effected in the liquid medium comprising at least part of the components forming the carrier system;
wherein a continuous method control has been carried out for monitoring at least one of the method status and the method progress comprising monitoring one or more method parameters selected from the group consisting of input of energy, input and removal of thermal energy, flow behavior, mass throughput, volume flows or combinations thereof; wherein method control has been effected by monitoring at least one process parameter selected from the group consisting of temperature, conductivity, viscosity and pH value of the liquid medium comprising the components forming the carrier system;
wherein the carrier system and the liquid medium are at least substantially free of ethanol.

181. The lipid-based carrier system according to claim 177,
wherein the lipid-based carrier system is characterized by at least one of the following features:
the substances (i) and (iii) or the substances (i) and (iv) or the substances (i) and (v) have an average HLB value according to Griffin in the range from 1 to 10;
the substances (i) and (iii) or the substances (i) and (iv) or the substances (i) and (v) comprise an average Log P value in the range from 0.62 to 8.58;
the substances (i), (iii) and (iv) or the substances (i), (iii) and (v) or the substances (i), (iv) and (v) have an average HLB value according to Griffin in the range from 1 to 10;
the substances (i), (iii) and (iv) or the substances (i), (iii) and (v) or the substances (i), (iv) and (v) comprise an average Log P value in the range from 0.62 to 8.58;
the substances (i), (iii), (iv) and (v) comprise an average HLB value according to Griffin in the range from 1 to 10;
the substances (i), (iii), (iv) and (v) comprise an average Log P value in the range from 0.62 to 8.58;
the substance (i) comprises a critical packing parameter Pₖᵣ of at least 0.5;
the substance (ii) comprises a critical packing parameter Pₖᵣ of at most 0.35;
the substance (iii) comprises a critical packing parameter Pₖᵣ of at least 1.155;
the substance (iv) comprises a critical packing parameter Pₖᵣ of at least 0.98;
the substance (v) comprises a critical packing parameter Pₖᵣ of at least 0.7.

182. The lipid-based carrier system according to claim 177,
wherein the carrier system is characterized by at least one of the following features (1b) to (5b):
(1b) the substance (i) comprises a critical packing parameter Pₖᵣ of at least 0.5;
(2b) the substance (ii) comprises a critical packing parameter Pₖᵣ of at most 0.35;
(3b) wherein the substance (iii) comprises a critical packing parameter Pₖᵣ of at least 1.155;
(4b) the substance (iv) comprises a critical packing parameter Pₖᵣ of at least 0.98;
(5b) the substance (v) comprises a critical packing parameter Pₖᵣ of at least 0.7.

183. The lipid-based carrier system according to claim 176,
wherein the active ingredient is a component of the carrier system and wherein the carrier system comprises the active ingredient

184. The lipid-based carrier system according to claim 178,
wherein the active ingredient is characterized by at least one of the following features:
the active ingredient is selected among therapeutic and prophylactic active ingredients and therapeutic and prophylactic pharmaceutical active ingredients and combinations thereof;
the active ingredient is selected from the group consisting of at least substantially water-insoluble active ingredients, at least substantially water-insoluble amphiphilic active ingredients, water-soluble active ingredients and combinations thereof;
the active ingredient is selected from the group consisting of vaccine active ingredients, antibiotic active ingredients, antifungal active ingredients, antiviral active ingredients, antiphlogistic active ingredients, antipyretic active ingredients, antiallergic active ingredients, anticancer active ingredients, hormone active ingredients, anticoagulant active ingredients, vitamins, minerals, UV-absorbing substances and combinations thereof;
the active ingredient is selected from the group consisting of RNA, retinol, tocopherol, corticosteroids, antibiotics, diclofenac, estradiol, estradiol hemihydrate, salicylic acid, acetylsalicylic acid, indomethacin, essential oils, amphiphilic UVA filters and UVB filters, nicotinamide, and combinations thereof;
the active ingredient is selected from the group consisting of vaccine active ingredients, nucleic acid-based vaccine active ingredients, gene-based vaccine active ingredients, vaccine active ingredients encoding an antigen of a pathogen or a pathogen, and combinations thereof;
the active ingredient is selected from the group consisting of vaccine active ingredients, wherein the vaccine active ingredients are selected from the group consisting of DNA, RNA, messenger RNA, viral vectors, plasmids, plasmid DNA and combinations thereof;
the active ingredient is selected from the group consisting of mRNA-based vaccine active ingredients and messenger RNA, and combinations thereof;
the active ingredient is selected from the group consisting of mRNA-based vaccine active ingredients and messenger RNA, and combinations thereof, wherein the mRNA encodes a protein of a coronavirus;
the active ingredient is selected from the group consisting of proteins and antigens and combinations thereof.

185. The lipid-based carrier system according to claim 177,
wherein the lipid-based carrier system is characterized by at least one of the following features:
agglomeration of the components has been effected such that the carrier system results in the form of lipid nanoparticles having the shape of micelles, vesicles or disc-shaped lipid nanoparticles;
the carrier system is in the form of lipid nanoparticles having the shape of disc-shaped lipid nanoparticles;
the particle sizes of the lipid nanoparticles of the carrier system comprise at least essentially a Gaussian distribution, wherein the particle sizes are determined using light diffraction;
the lipid nanoparticles of the carrier system have a particle size in the range from 5 nm to 10,000 nm, as determined using light diffraction;
the lipid nanoparticles of the carrier system have an average particle size in the range from 5 nm to 2,900 nm, as determined using light diffraction;
the variance of the particle size of the lipid nanoparticles of the carrier system is at most 45% in relation to the mean particle size, as determined using light diffraction;
the variance of the particle size of the lipid nanoparticles of the carrier system is in the range from 5% to 45% in relation to the mean particle size, as determined using light diffraction;
the lipid nanoparticles of the carrier system comprise an average particle size D50 in the range from 2.5 nm to 950 nm, as determined using light diffraction
the lipid nanoparticles of the carrier system comprise an average particle size D90 in the range from 1 nm to 1.000 nm, as determined using light diffraction.

186. The lipid-based carrier system according to claim 177,
wherein the lipid-based carrier system is characterized by at least one of the following features:
agglomeration of the components has been effected such that a liquid-crystalline structure on the basis of the carrier system and the liquid medium results;
the carrier system together with the liquid medium is present as a lyotropic liquid-crystalline structure based on the carrier system and the liquid medium;
the carrier system is contained in a liquid medium, wherein the carrier system forms a disperse composition with the liquid medium, wherein the carrier system is provided with at least one active ingredient;
the carrier system is contained in a liquid medium, wherein the carrier system forms a disperse composition with the liquid medium, wherein the composition comprises the lipid nanoparticles of the carrier system in an amount in the range from 5 wt. % to 95 wt. %;
the carrier system is contained in a liquid medium, wherein the carrier system forms a disperse composition with the liquid medium, wherein the carrier system is present in the composition in the form of lipid nanoparticles having the shape of micelles, vesicles or disc-shaped lipid nanoparticles.

187. The lipid-based carrier system according to claim 177,
wherein the lipid-based carrier system is characterized by at least one of the following features:
the carrier system is contained in a liquid medium, wherein the carrier system forms a disperse composition with the liquid medium, wherein the composition comprises a water activity in the range from 0 to 1, as determined according to at least one of ISO 21807:2004, ISO 18787:2017 and 21 CFR Part 11;
the carrier system comprises a total Log P value of at most 5;
the carrier system comprises a total Log P value in the range from 0.1 to 5;
the components of the carrier system are selected with the proviso that the resulting carrier system comprises a total Log P value of at most 5;
the components of the carrier system are selected with the proviso that the resulting carrier system comprises a total Log P value in the range from 0.1 to 5.

188. The lipid-based carrier system according to claim 177,
wherein the carrier system is contained in a liquid medium, wherein the carrier system forms a disperse composition with the liquid medium, wherein the liquid medium is selected such to be liquid at a temperature in the range from 20° C. to 40° C. at ambient pressure.

189. A drug or medicament for use in at least one of a prophylactic and therapeutic treatment of diseases of the human or animal body,
wherein the drug or medicament comprises at least one lipid-based carrier system according to claim 177 together with at least one pharmaceutical active ingredient.

190. The drug or medicament according to claim 189,
wherein the active ingredient is characterized by at least one of the following features:
the active ingredient is selected among therapeutic and prophylactic active ingredients and therapeutic and prophylactic pharmaceutical active ingredients and combinations thereof;
the active ingredient is selected from the group consisting of at least substantially water-insoluble active ingredients, at least substantially water-insoluble amphiphilic active ingredients, water-soluble active ingredients and combinations thereof;
the active ingredient is selected from the group consisting of vaccine active ingredients, antibiotic active ingredients, antifungal active ingredients, antiviral active ingredients, antiphlogistic active ingredients, antipyretic active ingredients, antiallergic active ingredients, anticancer active ingredients, hormone active ingredients, anticoagulant active ingredients, vitamins, minerals, UV-absorbing substances and combinations thereof;
the active ingredient is selected from the group consisting of RNA, retinol, tocopherol, corticosteroids, antibiotics, diclofenac, estradiol, estradiol hemihydrate, salicylic acid, acetylsalicylic acid, indomethacin, essential oils, amphiphilic UVA filters and UVB filters, nicotinamide, and combinations thereof;
the active ingredient is selected from the group consisting of vaccine active ingredients, nucleic acid-based vaccine active ingredients, gene-based vaccine active ingredients, vaccine active ingredients encoding an antigen of a pathogen or a pathogen, and combinations thereof;
the active ingredient is selected from the group consisting of vaccine active ingredients, wherein the vaccine active ingredients are selected from the group consisting of DNA, RNA, messenger RNA, viral vectors, plasmids, plasmid DNA and combinations thereof;
the active ingredient is selected from the group consisting of mRNA-based vaccine active ingredients and messenger RNA, and combinations thereof;
the active ingredient is selected from the group consisting of mRNA-based vaccine active ingredients and messenger RNA, and combinations thereof, wherein the mRNA encodes a protein of a coronavirus;
the active ingredient is selected from the group consisting of proteins and antigens and combinations thereof.

191. The drug or medicament according to claim 189,
wherein the drug or medicament further comprises at least one pharmaceutically acceptable excipient.
